(19) United States
(12) Patent Application Publication
(10) Pub. No.: US 2026/0364325 A1
(43) Pub. Date: Nov. 19, 2026

(54) SYSTEMS, METHODS, AND DEVICES FOR INFUSION OF A MATERIAL INTO A PATIENT

(71) Applicant: Fractyl Health, Inc., Burlington, MA (US)

(72) Inventors: Sara A. Morneau, Burlington, MA (US); Jay Caplan, Burlington, MA (US); Jacob Wainer, Malden, MA (US); Michael A. Biasella, Burlington, MA (US); Jason West, Lexington, MA (US); R. Maxwell Flaherty, Topsfield, MA (US); J. Christopher Flaherty, Nottingham, NH (US)

(73) Assignee: Fractyl Health, Inc., Burlington, MA (US)

(21) Appl. No.: 19/480,778

(22) PCT Filed: May 1, 2024
(86) PCT No.: PCT/US2024/027264

§ 371 Date: Oct. 31, 2025

Related U.S. Application Data
(60) Provisional application No. 63/499,456, filed on May 1, 2023.
(60) Provisional application No. 63/587,668, filed on Oct. 3, 2023.

(51) Int. Cl. A61M 37/00; A61M 25/00; A61M 25/01

(52) U.S. Cl. CPC A61M 37/00; A61M 25/0026; A61M 25/0045; A61M 25/0054; A61M 25/0127; A61M 2025/0034; A61M 2025/0046; A61M 2025/0057; A61M 2025/0166; A61M 2037/0007; A61M 2202/0007; A61M 2202/04; A61M 2205/0238; A61M 2205/3306; A61M 2205/3317; A61M 2205/3334; A61M 2205/3344; A61M 2205/3375; A61M 2205/3606; A61M 2205/502; A61M 2205/583; A61M 2205/8281; A61M 2210/1042

(57) ABSTRACT

Systems, devices, and methods for treating a medical condition of a patient are disclosed herein. The system includes a treatment material and a depositing device for delivering the treatment material to the patient. The depositing device includes: an infusion assembly having a shaft and a lumen therethrough; and a syringe assembly that provides the treatment material to the infusion assembly. The infusion assembly performs at least one infusion that includes: advancing the infusion assembly to a location proximate at least one deposit site in the patient; and delivering the treatment material into the at least one deposit site via the lumen of the shaft of the infusion assembly.

### CROSS-REFERENCE TO RELATED APPLICATIONS

This application claims priority to: U.S. Provisional Patent Application Ser. No. 63/499,456 (Attorney Docket No. 41714-729.101; Client Docket No. MCT-058-PR1), entitled “Systems, Methods, and Devices for Infusion of a Material into a Patient”, filed May 1, 2023; and U.S. Provisional Patent Application Ser. No. 63/587,668 (Attorney Docket No. 41714-729.102; Client Docket No. MCT-058-PR2), entitled “Systems, Methods, and Devices for Infusion of a Material into a Patient”, filed Oct. 3, 2023; the contents of each of which is incorporated herein by reference in its entirety for all purposes.

The subject matter of this application is related to that of: U.S. patent application Ser. No. 16/905,274 (Attorney Docket No. 41714-717.301; Client Docket No. MCT-036-US), entitled “Material Depositing System for Treating a Patient”, filed Jun. 18, 2020; U.S. patent application Ser. No. 17/214,157 (Attorney Docket No. 41714-718.301; Client Docket No. MCT-037-US), entitled “Systems and Methods for Deposition Material in a Patient”, filed Mar. 26, 2021; U.S. patent application Ser. No. 18/366,898 (Attorney Docket No. 41714-725.301; Client Docket No. MCT-053-US), entitled “System for Treating a Patient”, filed Aug. 8, 2023; U.S. patent application Ser. No. 18/634,104 (Attorney Docket No. 41714-725.302; Client Docket No. MCT-053-US-CON1), entitled “System for Treating a Patient”, filed Apr. 12, 2024; International PCT Patent Application Serial Number PCT/US2022/081543 (Client Docket No. MCT-080-PCT), entitled “Gene Therapies for Metabolic Disorders”, filed on Dec. 14, 2022; U.S. Provisional Patent Application Ser. No. 63/508,251 (Client Docket No. MCT-082-PR1), entitled “Gene Therapies for Metabolic Disorders”, filed Jun. 14, 2023; U.S. Provisional Patent Application Ser. No. 63/594,626 (Client Docket No. MCT-082-PR2), entitled “Gene Therapies for Metabolic Disorders”, filed Oct. 31, 2023; U.S. Provisional Patent Application Ser. No. 63/603,577 (Client Docket No. MCT-082-PR3), entitled “Gene Therapies for Metabolic Disorders”, filed Nov. 28, 2023; U.S. Provisional Patent Application Ser. No. 63/617,896 (Client Docket No. MCT-083-PR1), entitled “Gene Therapies for Metabolic Disorders”, filed Jan. 5, 2024; U.S. Provisional Patent Application Ser. No. 63/617,913 (Client Docket No. MCT-084-PR1), entitled “Combination Therapies for Metabolic Disorders”, filed Jan. 5, 2024; and U.S. patent application Ser. No. 18/630,579 (Client Docket No. MCT-081-US), entitled “Gene Therapies for Metabolic Disorders”, filed Apr. 9, 2024; the contents of each of which is incorporated herein by reference in its entirety for all purposes.

### TECHNICAL FIELD

The present invention relates generally to systems for treating a patient, in particular, to systems that deliver a material that provides a therapeutic benefit to the patient.

### BACKGROUND

The prevalence of metabolic and pancreatic diseases is growing worldwide. Specifically, Type 2 diabetes accounts for significant morbidity and mortality of the population, with an estimated 50 million people expected to be living with Type 2 diabetes in the United States by 2035. However, approximately 50% of the people diagnosed with Type 2 diabetes in the United States have inadequately controlled disease despite the availability of numerous diagnostic and therapeutic treatments.

For these and other reasons, there is a need for improved systems, methods, and devices for diagnosing and treating metabolic disease. There is an additional need for improved treatments of various diseases and disorders, such as treatment of diabetes.

### BRIEF SUMMARY

Embodiments of the systems, devices and methods described herein can be directed to systems, devices, and methods for the treatment of diabetes and similar patient diseases and disorders.

According to an aspect of the present inventive concepts, a system for treating a medical condition of a patient, comprises: a treatment material; and a depositing device for delivering the treatment material to the patient, the depositing device comprising: an infusion assembly comprising a shaft and a lumen therethrough, and a syringe assembly constructed and arranged to provide the treatment material to the infusion assembly. The infusion assembly is configured to perform at least one infusion comprising: advancing the infusion assembly to a location proximate at least one deposit site in the patient; and delivering the treatment material into the at least one deposit site via the lumen of the shaft of the infusion assembly. The system is configured to treat the medical condition of the patient.

In some embodiments, the medical condition comprises two or more medical conditions.

In some embodiments, the system is configured to treat one, two, three, or more medical conditions selected from the group consisting of: Type 2 diabetes; Type 1 diabetes; Double diabetes; gestational diabetes; other forms of diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; hyperglucagonemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor, stomach cancer; colorectal cancer; prostate cancer; diastolic dysfunction; hypertension; myocardial infarction; microvascular disease related to diabetes; anorexia nervosa; anorexia; a binge eating disorder, a hyperphagic state; hyperphagia; polyphagia; Prader Willi syndrome; an obesity-related genetic disorder; hypoglycemia; hypoglycemia that presents after a bariatric procedure; recurrent obesity post-bariatric surgery; recurrent metabolic disease post-bariatric surgery; iron overload conditions such as hemochromatosis types 1-4 and/or bantu siderosis; hypercholesterolemia; short bowel syndrome; sleep apnea; arthritis; rheumatoid arthritis; general lipodystrophy such as congenital, Berardinelli-Seip syndrome, acquired, and/or Lawrence syndrome; familial or acquired partial lipodystrophy such as Barraquer-Simons syndrome and/or Köbberling-Dunnigan syndrome; congenital leptin deficiency; lipoprotein lipase deficiency such as familial chylomicronemia syndrome, chylomicronemia, chylomicronemia syndrome, and/or hyperlipoproteinemia type Ia; Hemophilia A; Hemophilia B; Gaucher's disease; Fabry disease; Alpha-1 antitrypsin deficiency; galactosemia such as type 1, 2, and/or 3; Menkes disease; Wilson's disease; microvillus inclusion disease; congenital tufting enteropathy; chronic gastroparesis; eosinophilic intestinal disease; cystic fibrosis; exocrine pancreatic insufficiency; partial pancreatectomy; Crohn's disease; inflammatory bowel disease; eosinophilic esophagitis; Celiac disease; familial apolipoprotein E deficiency; aromatase deficiency; acute pancreatitis; chronic pancreatitis; pancreatic cancer; pancreatic metastases; congenital hyperinsulinism; and combinations thereof.

In some embodiments, the at least one deposit site comprises one or more sites within the pancreas of the patient.

In some embodiments, the system is configured to generate a localized distribution of the treatment material in a target region of the pancreas of the patient. The system can be further configured to distribute the treatment material to at least 5%, 10%, 15%, 20%, 30%, 40%, 50%, 60%, and/or 70% of cells in the targeted region. The targeted region can comprise a splenic lobe of the pancreas. The treatment material can comprise a treatment agent comprising one or more gene therapy materials.

In some embodiments, the one or more gene therapy materials comprise a gene therapy vector and/or a gene editing vector. The treatment material can comprise one, two, or more treatment agents selected from the group consisting of: an oncolytic virus; a cell therapy; an oligonucleotide such as an antisense oligonucleotide; an aptamer; small interfering RNA; microRNA; messenger RNA, an antibody such as a monoclonal antibody or bispecific antibody; an antibody-drug conjugate; a nanobody; and combinations thereof.

In some embodiments, the system is configured to deliver the treatment material to a target region, and the treatment material treats at least 20%, 30%, 40%, 50%, 60%, 70%, and/or 80% of cells in the target region. The system can be configured to deliver the treatment material to the target region in no more than five separate treatment material deliveries and/or no more than three separate treatment material deliveries. The treatment by the system does not result in the patient having pancreatitis. The treatment by the system has a risk of resulting in the patient having pancreatitis that is no more than 1.0%, no more than 0.5%, and/or no more than 0.1%. The treatment by the system does not result in elevation of one or more pancreatic enzymes above three times a normal level and/or no more than above two times a normal level. Any elevations in pancreatic enzymes can resolve in a time period of no more than 72 hours, no more than 48 hours, and/or no more than 24 hours. The treatment by the system does not result in elevation of lipase above three times the upper limit of normal.

In some embodiments, the system further comprises a positioning assembly constructed and arranged to introduce and position the infusion assembly in the patient.

In some embodiments, the syringe assembly is constructed and arranged to store the treatment material. The treatment material can be provided in the syringe assembly.

In some embodiments, the system further comprises a drive assembly constructed and arranged to operably attach to the syringe assembly and force the treatment material from the syringe assembly. The shaft of the infusion assembly can be constructed and arranged to transition from a first flexibility to a second flexibility after insertion into the pancreas, and the second flexibility can be more flexible than the first flexibility. The shaft of the infusion assembly can comprise a stiffening sheath constructed and arranged to surround the shaft, and the stiffening sheath can be configured to be removed from the shaft after insertion into the pancreas. The shaft of the infusion assembly can comprise a coating configured to increase the stiffness of the shaft, and the coating can be configured to rapidly degrade after insertion into the pancreas.

In some embodiments, the infusion assembly further comprises a visual indicator.

The shaft of the infusion assembly can be configured to be removed from one or more lumens of the system, and the visual indicator can be located on the shaft and configured to indicate to an operator when a pre-determined length of the shaft remains within the one or more lumens.

In some embodiments, the shaft of the infusion assembly comprises one or more fenestrations.

In some embodiments, the lumen of the shaft of the infusion assembly comprises a coating, and the coating comprises a surfactant.

In some embodiments, the infusion assembly is constructed and arranged to penetrate the pancreas axially.

In some embodiments, the shaft of the infusion assembly comprises a stepped profile configured to prevent a puncture into a tissue surface beyond 4 cm.

In some embodiments, the system further comprises an agent configured to be delivered to the patient. The agent can comprise a low-viscosity adhesive configured to be delivered to the patient prior to delivery of the treatment material. The agent can comprise a sealant configured to be delivered to the patient prior to the delivery of the treatment material.

In some embodiments, the system further comprises a functional element. The functional element can be configured to create an electric field proximate the deposit site. The electric field can be configured to electroporate the tissue prior to, during, and/or after the delivery of the treatment material.

In some embodiments, the system further comprises a navigational sensor configured to determine the position of at least a portion of the infusion assembly. The navigational sensor can comprise a stylet. The navigational sensor can comprise an electromagnetic position sensor. The navigational sensor can comprise a fiberoptic shape sensing sensor.

In some embodiments, the depositing device is constructed and arranged to be robotically controlled and/or manipulated.

In some embodiments, the depositing device is configured to access the pancreas via a percutaneous approach. The percutaneous approach can comprise an ultrasound-guided percutaneous approach. The percutaneous approach can comprise a CT-guided percutaneous approach.

In some embodiments, the delivery of the treatment material comprises pressure-controlled delivery of the treatment material.

In some embodiments, the delivery of the treatment material comprises flow-controlled delivery of the treatment material.

In some embodiments, the system further comprises a console constructed and arranged to display a graphical user interface to an operator. The system can further comprise an imaging device configured to capture one or more images. The graphical user interface can be configured to display the one or more images captured by the imaging device and to display one or more overlays on the one or more images. The one or more overlays can each indicate a projected path of the infusion device.

In some embodiments, the shaft of the infusion assembly comprises a coating. The coating can comprise a hyperechogenic coating.

According to another aspect of the present inventive concepts, a method for treating a medical condition of a patient, comprises selecting a system as claimed in any one or more claims herein; advancing the infusion assembly to a location proximate at least one deposit site in the patient; and delivering the treatment material into the at least one deposit site via the infusion assembly. The method treats one or more medical conditions of the patient.

In some embodiments, the at least one deposit site comprises one or more sites in the pancreas; and the infusion assembly comprises a distal portion comprising a needle with a diameter of no more than 23 G and/or no more than 25 G; and the needle is inserted into the parenchyma of the pancreas at least one time, at least two times, and/or at least three times; and the treatment material is delivered at a controlled flow rate. The method can result in transient elevations in pancreatic enzymes that are below three times and/or below two times a normal upper level for the pancreatic enzymes. The elevations in pancreatic enzymes can return to a normal value in less than 72 hours, less than 48 hours, and/or less than 24 hours. The controlled flow rate can comprise a flow rate of no more than 20 mL/min, 15 mL/min, 10 mL/min, 5 mL/min, 3 mL/min, and/or 1 mL/min. The maximum number of separate treatment material deliveries can be no more than 5, 4, 3, and/or 2 deliveries. The system can be configured to generate a localized distribution of the treatment material in a target region of the pancreas of the patient, and the target region can comprise a portion of the pancreas and/or one or more cell types of the pancreas. The system can be configured to generate a localized distribution of the treatment material in a target region of the pancreas of the patient, and the target region can comprise the head of the pancreas, the neck of the pancreas, the body of the pancreas, and/or the tail of the pancreas. The target region can comprise at least the body of the pancreas and the tail of the pancreas. The system can be configured to generate a localized distribution of the treatment material in a target region of the pancreas of the patient, and the target region can comprise a region of the pancreas containing malignant tissue and/or cancerous tissue. The system can be configured to generate a localized distribution of the treatment material in a target region of the pancreas of the patient, and the target region can comprise a region of the pancreas comprising a cell type comprising malignant cells and/or cancerous cells. The system can be configured to generate a localized distribution of the treatment material in a target region of the pancreas of the patient, and the target region can comprise a region of the pancreas comprising one, two, or more cell types selected from the group consisting of: exocrine cells such as acinar cells; duct cells and/or other exocrine cells; endocrine cells such as alpha cells, beta cells, delta cells, epsilon cells, pp cells, G cells, and/or other endocrine cells; a type of vascular cell, such as endothelial cells, pericytes, smooth muscle cells, and/or other vascular cells; a type of immune cell, such as macrophages, mast cells, T cells, B cells, lymphocytes, dendritic cells, and/or other types of immune cells; and combinations thereof. The system can be configured to generate a localized distribution of the treatment material in a target region of the pancreas of the patient, and the treatment material can be delivered to at least 10%, 20%, 30%, 40%, or 50% of the target region. The system can be configured to generate a localized distribution of the treatment material in a target region of the pancreas of the patient, and the treatment material can be delivered to no more than 30%, 40%, or 50% of the target region. The system can be configured to avoid a non-target region of the pancreas. The system can be configured to limit systemic distribution of the treatment material. The limited systemic distribution can comprise a level that is below a toxic level to one or more non-pancreatic organs. The system can be configured to provide systemic distribution of the treatment material. The system can be configured to cause a systemic distribution of the treatment material at a level that provides a therapeutic dose to one or more non-pancreatic organs.

In some embodiments, the drive assembly comprises a housing, a constant force spring, and a translating assembly, and the constant force spring applies a translating force to the translating assembly. The constant force spring can be wound about a portion of the translating assembly. The housing can comprise a projection, and the constant force spring can be wound about the projection. The drive assembly can be configured to be reset by manually retracting the translating assembly against the force of the constant force spring.

The drive assembly can further comprise a cranking assembly configured to retract the translating assembly. The drive assembly can further comprise a locking assembly configured to lock the translating assembly in a retracted position. The locking assembly can comprise a ratcheting lock. The locking assembly can comprise a sliding latch.

In some embodiments, the shaft of the infusion assembly comprises a stepped profile configured to prevent backflow of the treatment material. The shaft of the infusion assembly can comprise a first outer diameter and the stepped profile can comprise a second outer diameter, and the second outer diameter can be at least 0.001″ greater than the first diameter. The system can further comprise an over tube positioned over the shaft of the infusion assembly, and the over tube can form the stepped profile. The system can further comprise an adhesive bead applied to the shaft of the infusion assembly, and the adhesive bead can form the stepped profile. The stepped profile can comprise a reduced diameter portion of the shaft of the infusion assembly, and the reduced diameter portion can be constructed and arranged to allow the treatment material to pool. The stepped profile can be at least 0.5 cm from a distal end of the shaft of the infusion assembly. The stepped profile can be no more than 1.5 cm from a distal end of the shaft of the infusion assembly.

In some embodiments, the system comprises a dead volume comprising a volume of the treatment material that is not delivered to the patient. The syringe assembly can comprise a low-dead volume syringe comprising a dead volume of less than 50 μL, such as less than 30 μL. The infusion assembly can comprise a lumen comprising an inner diameter, and the inner diameter can comprise a diameter of no more than 0.260 mm, such as no more than 0.210 mm. The system can comprise no more than two fluid connectors between the syringe assembly and the infusion assembly, such as no more than one fluid connector.

In some embodiments, the system further comprises a functional element configured to modify the delivery of the treatment agent to the deposit site. The functional element can comprise an element selected from the group consisting of: a vacuum element; a heating element; a cooling element; an iontophoretic element; and combinations thereof.

In some embodiments, the system further comprises a stylet configured to be removably positioned within the shaft of the infusion assembly, the shaft of the infusion assembly comprises a relatively flexible material, and the stylet comprises a relatively stiff material. The shaft of the infusion assembly can comprise soft plastic material.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

The foregoing and other objects, features, and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 illustrates a schematic view of a system for depositing a material at a deposit site of a patient, consistent with the present inventive concepts.

FIG. 2 illustrates a method of treating a patient while limiting transient physiologic response, consistent with the present inventive concepts.

FIGS. 2A and 2B illustrate graphs of pancreatic enzyme levels monitored during an animal study performed by the applicant, consistent with the present inventive concepts.

FIG. 3 illustrates an anatomic view of a portion of a patient's GI tract and pancreas, consistent with the present inventive concepts.

FIGS. 4A-4F illustrate various sectional and perspective views of a depositing device, consistent with the present inventive concepts.

FIGS. 5A and 5B illustrate sectional views of a “needle in needle” configuration of a delivery device, consistent with the present inventive concepts.

FIGS. 6A and 6B illustrate sectional views of an infusion device with an extendable needle tip, consistent with the present inventive concepts.

FIG. 7 illustrates a representative image of the pancreatic anatomy to be displayed to an operator, consistent with the present inventive concepts.

FIGS. 8A-8C illustrate perspective and side sectional views of a depositing device, consistent with the present inventive concepts.

FIG. 9 illustrates a sectional view of a locking mechanism of a deployment assembly, consistent with the present inventive concepts.

FIGS. 10A and 10B illustrate sectional views of a rack and pinion locking mechanism, consistent with the present inventive concepts.

FIGS. 11, 11A, and 11B illustrate sectional views of various components of a syringe and a syringe drive assembly including a spring and a damper, consistent with the present inventive concepts.

FIG. 12 illustrates a sectional view of a syringe drive assembly including a pull wire mechanism, consistent with the present inventive concepts.

FIG. 13 illustrates a sectional view of a syringe drive assembly including a rack and pinion mechanism, consistent with the present inventive concepts.

FIG. 14 illustrates side views of the handle portion of a depositing device, consistent with the present inventive concepts.

FIGS. 15A and 15B illustrate perspective views of a syringe drive assembly, consistent with the present inventive concepts.

FIG. 16 illustrates a sectional view of a syringe drive assembly, consistent with the present inventive concepts.

FIG. 17 illustrates a sectional view of a syringe drive assembly, consistent with the present inventive concepts.

FIG. 18 illustrates a sectional view of a material storage and delivery assembly, consistent with the present inventive concepts.

FIG. 19 illustrates a sectional view of a material storage and delivery assembly, consistent with the present inventive concepts.

FIGS. 20A and 20B illustrate side views of a material storage and delivery assembly, consistent with the present inventive concepts.

FIGS. 21, 21A, and 21B illustrate a photograph of the distal portion of a shaft including a hyperechogenic coating, and examples of uncoated and coated needles as viewed using ultrasonic imaging, respectively, consistent with the present inventive concepts.

FIG. 22 illustrates a sectional view of an embodiment of an infusion assembly, consistent with the present inventive concepts.

FIG. 23 illustrates an anatomic view of an embodiment of an infusion device inserted into tissue, consistent with the present inventive concepts.

FIGS. 24 and 24A illustrate a schematic view of an embodiment of a drive assembly, and a photograph of the drive assembly, respectively, consistent with the present inventive concepts.

FIGS. 25A-25D illustrate various embodiments of infusion assemblies comprising stepped profiles, consistent with the present inventive concepts.

FIG. 26 illustrates a method of limiting backflow during delivery of an injectate under visualization, consistent with the present inventive concepts.

FIGS. 27A-27C illustrate various embodiments of an infusion assembly, consistent with the present inventive concepts.

FIG. 28 illustrates a side view of the distal end of an embodiment of a syringe plunger, consistent with the present inventive concepts.

FIG. 29 illustrates a sectional view of an embodiment of an infusion assembly comprising a multi-part configuration, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element (also referred to as a “component” herein) is described as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

As used herein, the terms “operably attached”, “operably connected”, “operatively coupled” and similar terms related to attachment of components shall refer to attachment of two or more components that results in one, two, or more of: electrical attachment; fluid attachment; magnetic attachment; mechanical attachment; optical attachment; sonic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g., within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g., a target tissue and/or deposit site location), shall include components positioned near to the anatomical site, as well as components positioned in, on and/or within the anatomical site.

Spatially relative terms, such as “beneath,” “below,” “lower,” “above,” “upper”, “under” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A, one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±20% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g., efficacious therapy) and/or to prevent or otherwise reduce (hereinafter “prevent”) an undesired event (e.g., a device and/or clinical adverse event). In some embodiments, a system parameter is maintained above a first threshold (e.g., above a first temperature threshold to cause a desired therapeutic effect to tissue) and below a second threshold (e.g., below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy. In some embodiments, a functional element is configured to treat tissue (e.g., a functional element configured as a treatment element). Alternatively or additionally, a functional clement (e.g., a functional clement comprising a sensor) can be configured to record one or more parameters, such as a patient physiologic parameter; a patient anatomical parameter (e.g., a tissue geometry parameter); a patient environment parameter; and/or a system parameter. In some embodiments, a sensor or other functional element is configured to perform a diagnostic function (e.g., to gather data used to perform a diagnosis). In some embodiments, a functional element is configured to perform a therapeutic function (e.g., to deliver therapeutic energy and/or a therapeutic agent). In some embodiments, a functional element comprises one or more elements constructed and arranged to perform a function selected from the group consisting of: deliver energy; extract energy (e.g., to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input and produces an output. For example, a transducer can include an electrode that receives electrical energy and distributes the electrical energy to tissue (e.g., based on the size of the electrode). In some configurations, a transducer converts an electrical signal into any output, such as: light (e.g., a transducer comprising a light emitting diode or light bulb), sound (e.g., a transducer comprising a piezo crystal configured to deliver ultrasound energy); pressure (e.g., an applied pressure or force); heat energy; cryogenic energy; chemical energy; mechanical energy (e.g., a transducer comprising a motor or a solenoid); magnetic energy; and/or a different electrical signal (e.g., different than the input signal to the transducer). Alternatively or additionally, a transducer can convert a physical quantity (e.g., variations in a physical quantity) into an electrical signal. A transducer can include any component that delivers energy and/or an agent to tissue, such as a transducer configured to deliver one or more of: electrical energy to tissue (e.g., a transducer comprising one or more electrodes); light energy to tissue (e.g., a transducer comprising a laser, light emitting diode and/or optical component such as a lens or prism); mechanical energy to tissue (e.g., a transducer comprising a tissue manipulating element); sound energy to tissue (e.g., a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Obesity and Type 2 Diabetes are twin epidemics often associated with a Western Diet and sedentary lifestyle, and both of which are increasing in prevalence at startling rates. A normal endocrine pancreas produces insulin in response to glucose intake, but in patients with Type 2 diabetes, the insulin production capacity of the pancreas is significantly reduced and is no longer sufficient to control blood glucose.

Type 1 Diabetes is also a disease of the pancreas, in which the patient's immune system attacks the insulin-producing beta cells within the pancreas, leading to significantly reduced insulin production capacity and insufficient control of blood glucose.

Cystic fibrosis (CF) is the most common genetic disorder and causes significant damage in secretory epithelial cells due to mutations in the CTFR gene. CF in the pancreas can cause pancreatic exocrine insufficiency and consequent digestive dysfunction in the intestine, abdominal pain, an increased risk of acute pancreatitis, and a significant decrease in the quality of life.

Ex vivo gene therapy techniques have been tested in non-human animal models to attempt to improve pancreatic function, for example to cause transgenic overexpression of hepatocyte growth factor in the endocrine pancreas to stimulate insulin production. The ex vivo approach requires a complicated removal of pancreatic cells, ex vivo transduction, and reimplantation. Intraperitoneal injection of gene therapy agents is known to enable gene therapy in rodent models, as the pancreas is accessible to the peritoneal cavity. However, in humans, the pancreas is not accessible to the peritoneal cavity. To date, the rate of gene therapy transduction of the pancreas with an intravenous delivery of gene therapy agents has been shown to be well below therapeutic thresholds.

According to the American Cancer Society, the five-year survival rate for localized pancreatic cancer is 39%. Chemotherapy, using an intravenous infusion of gemcitabine or other agents, is an established therapy for pancreatic cancer. However, dosage is limited by systemic delivery and consequent off-target effects. Local drug delivery systems, such as gemcitabine-eluting membranes, have been implanted in the pancreas of mice as a means of providing local delivery of chemotherapeutic agents, but this requires both an implantable device and a complicated pancreatic surgery.

Pancreatitis is inflammation of the pancreas that can worsen over time and lead to permanent damage to the pancreas. Chronic pancreatitis eventually impairs a patient's ability to digest food and produce pancreatic hormones. Intra-pancreatic trypsinogen activation is believed to contribute to the pathogenesis of both acute and chronic pancreatitis. Trypsin inhibitors have demonstrated effectiveness in mouse models of pancreatitis. However, systemic trypsin inhibition is known to interfere with normal digestion, leading to malnutrition, delayed growth, and other metabolic and digestive disorders.

A total pancreatectomy with intraportal islet cell autotransplant is increasingly being offered to patients with chronic pancreatitis (CP). Intraportal delivery of pancreatic islets is an established therapy to restore some endocrine function to patients that have received a total pancreatectomy. Currently, the intraportal islet cell autotransplant procedure is performed with either a surgical approach or a transabdominal percutaneous approach. Complications of the current approaches include bleeding, thrombosis, and iatrogenic injury to anatomical structures.

Provided herein are systems and methods for delivering, infusing, implanting, placing, seeding, inserting, spraying, topically applying, and/or otherwise depositing (“depositing”, “infusing”, or “delivering” herein) a treatment material (e.g., a material including one or more treatment agents, treatment material 60 described herein) at one or more “deposit sites” of a patient (e.g., one or more internal locations of a mammalian patient, as described herein), such as to provide a therapeutic benefit to the patient. The systems and methods can be configured to treat a “medical condition” of the patient, such as one, two, three or more diseases, disorders, and/or other medical conditions of a patient. The system of the present inventive concepts, system 10, includes a depositing device, device 100, for the depositing of the treatment material, material 60, at the one or more deposit sites. Treatment material 60 can be deposited at one or more deposit sites to modify and/or otherwise generate desired resultant tissue in one or more locations, such as when depositing treatment material 60 into tissue of the pancreas and/or other tissue location to transduce tissue in those locations. Material 60 can comprise a treatment agent comprising a gene therapy material such as a material comprising a gene therapy vector (e.g., a gene therapy vector which carries and adds a new transgene to a cell) and/or a gene editing vector (e.g., a gene editing vector which carries a gene editing sequence and modifies an existing gene in a cell). In some embodiments, treatment material 60 comprises both a gene therapy vector and a gene editing vector. In some embodiments, a treatment material 60 comprising a gene editing vector comprises a virus-based, a lipid nanoparticle-based, and/or a plasmid-based vector.

Technologies used for gene editing can include zinc finger nucleases (ZFNs), transcription activator-like effector nucleases (TALENs), and/or “clustered regularly interspaced short palindromic repeats” (Crispr) based technologies. Treatment material 60 can comprise a treatment agent comprising: an oncolytic virus; a cell therapy; an oligonucleotide such as an antisense oligonucleotide; an aptamer; small interfering RNA; microRNA; messenger RNA; an antibody such as a monoclonal antibody or bispecific antibody, an antibody-drug conjugate; and/or a nanobody. In some embodiments, treatment material 60 comprises pancreatic cells, such as pancreatic beta cells, pancreatic alpha cells, or other pancreatic cells. Treatment material 60 can comprise pancreatic islets, such as donor and/or hypoimmune pancreatic islets. Treatment material 60 can comprise one, two, or more materials such as is described in reference to applicant's: co-pending U.S. patent application Ser. No. 18/630,579 (Client Docket No. MCT-081-US), entitled “Gene Therapies for Metabolic Disorders”, filed Apr. 9, 2024; and co-pending International PCT Patent Application Serial Number PCT/US2022/081543 (Client Docket No. MCT-080-PCT), entitled “Gene Therapies for Metabolic Disorders”, filed on Dec. 14, 2022.

The deposited treatment material 60 “modifies” tissue (e.g., modifies existing tissue and/or causes new tissue to be generated), and the modified tissue is configured to treat the medical condition of the patient. The systems of the present inventive concepts can be configured to maximize efficacy of the delivery of one or more materials to each deposit site (e.g., target tissue) of a patient, while minimizing patient risk as well as preventing or at least reducing the delivery of the one or more materials to undesired (e.g., non-target) tissue locations. In some embodiments, a treatment agent, such as an agent comprising at least a gene therapy material, is delivered into the pancreas of a patient to provide a therapeutic benefit to the patient. The treatment material can be delivered to at least 20% of the volume of the pancreas (e.g., the volume of the pancreatic tissue), and the treatment material can be delivered at a relatively slow flow rate. Therapeutic benefit can be achieved with minimal risk of pancreatitis, and while avoiding any significant distribution of the treatment agent to non-target organs.

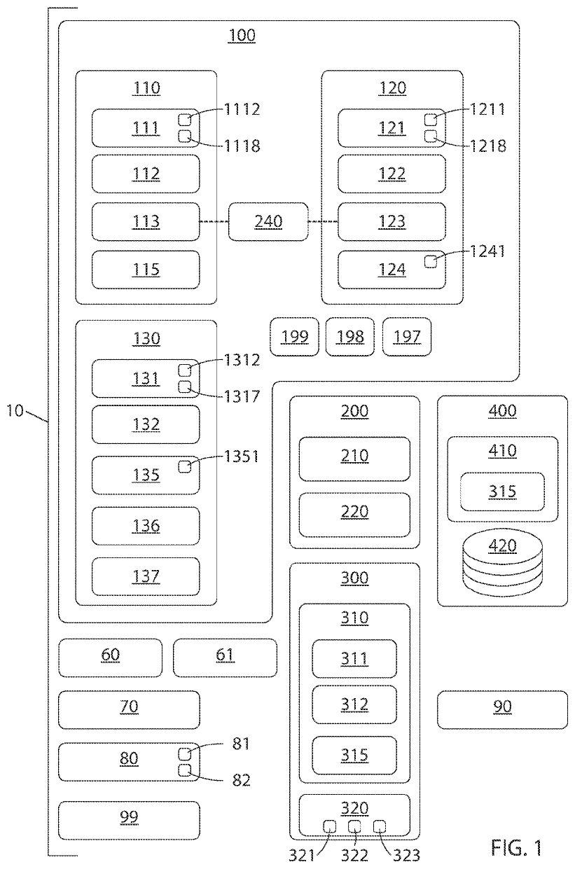

Referring now to FIG. 1, a schematic view of a system for depositing a material at a deposit site of a patient is illustrated, consistent with the present inventive concepts. System 10 includes one or more depositing devices, depositing device 100 shown. Depositing device 100 can comprise one or more devices for performing a treatment procedure comprising delivering, infusing, implanting, placing, seeding, inserting, spraying, topically applying, and/or otherwise depositing (“depositing” herein) one or more treatment agents, treatment material 60 shown and described herein, at a “deposit site” of a patient. The deposit site can comprise one, two, or more sites on and/or within a patient (e.g., on the patient's skin and/or within the body of the patient, respectively). After the depositing of treatment material 60, tissue at and/or proximate the deposit site, “target tissue” herein, can be modified by treatment material 60. As used herein, a “target region” can include a region of tissue comprising target tissue and/or other tissue, but does not comprise tissue to which treatment should be avoided, non-target tissue, described herein. System 10 can be configured to cause material 60 to enter one or more target regions (each target region including target tissue to be treated), such as by delivering material 60 to a corresponding one or more deposit sites (e.g., where material 60 enters each target region via the delivery of material 60 to the one or more deposit sites). In some embodiments, depositing device 100 delivers a material (e.g., material 60) into one, two, three, or more deposit sites, such as to effectively deliver the material to the target tissue within the target region to cause a desired effect (e.g., a desired therapeutic effect). Types of modifications of target tissue caused by depositing of treatment material 60 can include but are not limited to: creation of new tissue; gene modification and/or other modification of existing tissue; treatment of tissue with a pharmaceutical or other agent (e.g., treatment with an anti-pancreatitis pharmaceutical agent); destruction of tissue (e.g., via a chemotherapy agent); other modification of tissue; and combinations of these. The modified tissue (e.g., tissue including deposited treatment material 60 and/or tissue void of treatment material 60) comprises “resultant tissue” herein. Properties of the resultant tissue can be driven by or otherwise based on one or more effects of treatment material 60 on tissue (e.g., properties including expression of one or more proteins by the resultant material). Generation of the resultant tissue by the systems, devices, and methods of the present inventive concepts can provide a therapeutic benefit, such as an improvement in one or more medical conditions of the patient.

In some embodiments, material 60 includes one or more additives, such as additive 61 shown. Additive 61 can be configured to ease the delivery of material 60 to the patient, and/or to protect the integrity (e.g., prevent the degradation) of material 60 during storage and/or during a depositing procedure. For example, additive 61 can be configured to limit viral loss, such as via viral adsorption, as described herein.

System 10 and the methods of the present inventive concepts can treat one, two, three, or more medical conditions selected from the group consisting of: Type 2 diabetes; Type 1 diabetes; Double diabetes; gestational diabetes; other forms of diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease (NAFLD); non-alcoholic steatohepatitis (NASH); metabolic dysfunction-associated steatotic liver disease (MASLD); metabolic dysfunction-associated steatohepatitis (MASH); obesity; obesity-related disorder; polycystic ovarian syndrome; hypertriglyceridemia; hypercholesterolemia; hyperglucagonemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart discase; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder, a hyperphagic state; hyperphagia; polyphagia; Prader Willi syndrome; an obesity-related genetic disorder; hypoglycemia; hypoglycemia that presents after a bariatric procedure (referred to as “post-bariatric hypoglycemia”); recurrent obesity post-bariatric surgery; recurrent metabolic disease post-bariatric surgery; iron overload conditions such as hemochromatosis types 1-4 and/or bantu siderosis; hypercholesterolemia; pancreatic cancer; short bowel syndrome; sleep apnea; arthritis; rheumatoid arthritis; general lipodystrophy (e.g., congenital, Berardinelli-Seip syndrome, acquired, and/or Lawrence syndrome); familial or acquired partial lipodystrophy (e.g., Barraquer-Simons syndrome and/or Köbberling-Dunnigan syndrome); congenital leptin deficiency; lipoprotein lipase deficiency (e.g., familial chylomicronemia syndrome, chylomicronemia, chylomicronemia syndrome, and/or hyperlipoproteinemia type Ia); Hemophilia A; Hemophilia B; Gaucher's disease; Fabry disease; Alpha-1 antitrypsin deficiency; galactosemia (e.g., type 1, 2, and/or 3); Menkes disease; Wilson's disease; microvillus inclusion disease; congenital tufting enteropathy; chronic gastroparesis; eosinophilic intestinal disease; cystic fibrosis; exocrine pancreatic insufficiency; partial pancreatectomy; Crohn's disease; inflammatory bowel disease (IBD); eosinophilic esophagitis; Celiac disease; familial apolipoprotein E deficiency; aromatase deficiency; acute pancreatitis; chronic pancreatitis; pancreatic metastases; congenital hyperinsulinism; and combinations of these. In some embodiments, system 10 is configured to treat at least two, or at least three of the above medical conditions.

In some embodiments, the patient has Type 1 or Type 2 diabetes, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has double diabetes, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has gestational diabetes, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has hyperglycemia, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has pre-diabetes, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has monogenic diabetes, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponcctin, cholccystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has maturity onset diabetes of the young, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has impaired glucose tolerance, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1,insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has insulin resistance, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has hyperinsulinemia, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has hypoinsulinemia and/or relative beta cell insulin-production failure, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has non-alcoholic fatty liver disease (NAFLD); non-alcoholic steatohepatitis (NASH), metabolic dysfunction-associated steatotic liver disease (MASLD); metabolic dysfunction-associated steatohepatitis (MASH); and/or obesity; and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient is obese and/or has an obesity related disorder, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has polycystic ovarian syndrome (PCOS), and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has hypertriglyceridemia, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has hypercholesterolemia, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has diabetic nephropathy, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has retinopathy, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has heart disease, such as diabetic heart disease and/or heart failure, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has coronary artery disease or a history of stroke and/or transient ischemic attack, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

In some embodiments, the patient has cognitive decline and/or dementia and/or Alzheimer's disease, and a treatment of the present inventive concepts can be performed using system 10 to modify pancreatic tissue (e.g., pancreatic islet cells) in order to achieve one or more of the following effects: produce and/or secrete one or more hormones or hormone receptor agonists, such as GLP-1, insulin, GIP, glucagon, amylin, PYY, oxyntomodulin, adiponectin, cholecystokinin, estradiol, ghrelin, GLP-2 and/or leptin. In some embodiments the production and/or secretion may be constitutive and/or the production and/or secretion may be regulated, such as nutrient-responsive regulation.

System 10, including depositing device 100 and material 60, can be configured to avoid effecting certain volumes of tissue of the patient, “non-target tissue” herein. For example, depositing device 100 can be configured to prevent or at least reduce delivery of treatment material 60 to an undesired location, referred to as “non-target region” herein, such as a region that includes non-target tissue that is on the patient (e.g., skin) and/or within the patient, such as to avoid undesired effects that treatment material 60 may have on the non-target tissue. In some embodiments, depositing device 100 is configured to minimize distribution of treatment material 60 to one or more non-target regions (e.g., one or more “non-target organ” locations), as described herein. Depositing device 100 and/or other components of system 10 can also be configured to avoid passing through (e.g., and undesirably damaging) certain tissue, also “non-target tissue” herein, such as certain blood vessels, ducts, organ tissue, and the like (e.g., tissue whose modification by system 10 is not desired).

In some embodiments, system 10 and the devices and methods of the present inventive concepts are of similar construction and arrangement, and medical conditions treated are the same or similar, to those described in applicant's co-pending U.S. patent application Ser. No. 18/366,898 (Attorney Docket No. 41714-725.301; Client Docket No. MCT-053-US), entitled “System for Treating a Patient”, filed Aug. 8, 2023.

Depositing device 100 can comprise one or more similar and/or dissimilar depositing devices. For example, a first device 100 and a second device 100 can comprise a difference selected from the group consisting of: different degree of curvature; different length; different diameter; different patterns of fenestrations; different echogenicity pattern (e.g., solid versus stripes or spiral echogenic portion), and combinations of these. Depositing device 100 of system 10 can include one or more assemblies for infusing treatment material 60 at a deposit site, infusion assembly 110 shown. Infusion assembly 110 can include shaft 111 with proximal portion 1112 and distal end 1118, and one or more lumens, lumen 112, extending between proximal portion 1112 (e.g., from the proximal end of shaft 111) and distal end 1118. Distal end 1118 of shaft 111 is to be positioned proximate a deposit site during an infusion or other delivery (“infusion”, “depositing”, or “delivery”, and the like herein) of treatment material 60. Infusion assembly 110 can include a connector, connector 113, located near proximal portion 1112 of shaft 111, and fluidly connected to lumen 112.

Depositing device 100 of system 10 can include one or more syringe assemblies, syringe assembly 120 shown, each syringe assembly 120 comprising one or more syringes. A syringe assembly 120 can be constructed and arranged for storing a material (e.g., treatment material 60 or a material including treatment material 60) to be deposited into the patient (e.g., immediately prior to infusion) and/or for providing the material to infusion assembly 110. Syringe assembly 120 can include an elongate body, barrel 121, with proximal end 1211 and distal end 1218, and a chamber extending therebetween, chamber 122. In some embodiments, syringe assembly 120 comprises connector 123 located on distal end 1218 of barrel 121 and fluidly connected to chamber 122. Connector 123 can be configured to fluidly attach to connector 113 of infusion assembly 110, such that a material (e.g., treatment material 60) can flow from chamber 122 through lumen 112 of infusion assembly 110, such as to be delivered into the patient. In some embodiments, depositing device 100 includes one or more fluid conduits, extension tube 240, that provides a fluid connection between syringe assembly 120 and infusion assembly 110. For example, extension tube 240 can fluidly connect, on a first end via a first connector, to connector 123 of syringe assembly 120, and on a second end via a second connector, to connector 113 of infusion assembly 110. Syringe assembly 120 can include a material drive mechanism, plunger 124, that is slidingly received within chamber 122 and is configured to drive (e.g., push) material from chamber 122 (e.g., into infusion assembly 110). Plunger 124 can comprise a sealing drive element, seal 1241, that is slidingly received within chamber 122 and forms a fluidic seal with the walls of chamber 122. Plunger 124 can be pulled proximally (e.g., when seal 1241 begins at a distal most position within chamber 122) to draw material (e.g., treatment material 60) into chamber 122, and/or plunger 124 can be pushed distally to force material from chamber 122 (e.g., through connector 123). In some embodiments, plunger 124 can be inserted and/or removed from barrel 121, such as to allow material to be loaded into chamber 122 from proximal end 1211 of barrel 121.

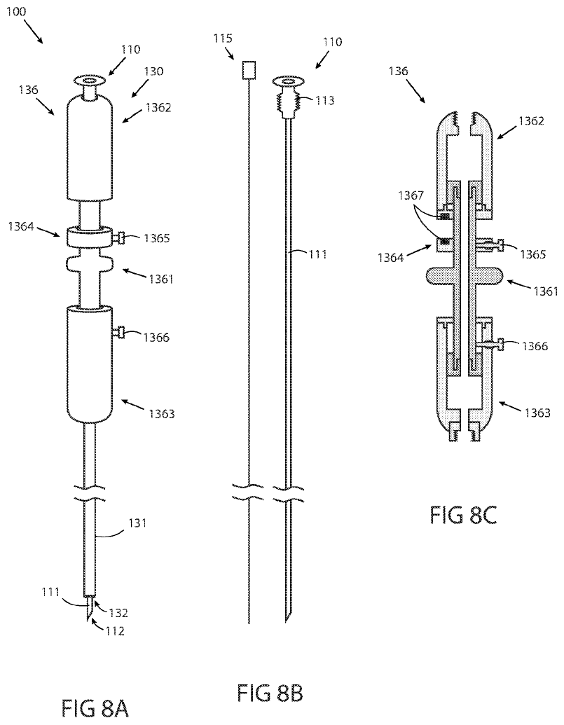

Depositing device 100 of system 10 can include an assembly for introducing and positioning infusion assembly 110 within the patient, positioning assembly 130 shown. Positioning assembly 130 can include one or more elongate bodies, shaft 131, which includes proximal portion 1312 and distal portion 1317, and one or more lumens extending therebetween, lumen 132, each as shown. Lumen 132 can be constructed and arranged to slidingly receive at least a portion of shaft 111 of infusion assembly 110. In some embodiments, positioning assembly 130 comprises articulation control assembly 135 that is constructed and arranged to control the shape and/or position of positioning assembly 130, for example when articulation control assembly 135 comprises one or more steering cables configured to manipulate distal portion 1317 of shaft 131). Positioning assembly 130 can include deployment control assembly 136 that temporarily fixes at least a portion of infusion assembly 110 to positioning assembly 130 and allows the operator to deploy at least a portion of infusion assembly 110 from the distal end of lumen 132. In some embodiments, deployment control assembly 136 is constructed and arranged as described in reference to FIGS. 8A-C and otherwise herein. In some embodiments, positioning assembly 130 includes an assembly comprising one or more imaging components, imaging assembly 137. For example, imaging assembly 137 can comprise an ultrasonic imaging assembly, such as when device 100 is configured to perform endoscopic procedures with ultrasonic guidance (e.g., when positioning assembly 130 comprises an endoscopic ultrasound device, “EUS” or “EUS device” herein). Alternatively or additionally, imaging assembly 137 can comprise a camera, such as a visible light and/or infrared camera.

In some embodiments, system 10 includes drive assembly 200 as shown, an assembly that is constructed and arranged to operably attach to syringe assembly 120, and drives (e.g., translates) plunger 124 within chamber 122, forcing material within chamber 122 to exit chamber 122 (e.g., into lumen 132 of infusion assembly 110 and into the patient as plunger 124 is advanced). Drive assembly 200 can include an adapter, syringe adaptor 210 shown, that removably attaches syringe assembly 120 to drive assembly 200. Drive assembly 200 can also include a translating or other force applying mechanism, motive assembly 220, that operably attaches to plunger 124 of syringe assembly 120 and can drive plunger 124 to translate distally and/or proximally relative to barrel 121 (e.g., into and/or out of chamber 122).

In some embodiments, system 10 includes one or more consoles, console 300 shown. Console 300 can operably attach (e.g., fluidly, electrically, magnetically, mechanically, pneumatically, hydraulically, optically, and/or acoustically attach) to device 100 and/or other devices or assemblies of system 10. In some embodiments, console 300 comprises drive assembly 200 (e.g., one or more housings of console 300 surround all or a portion of drive assembly 200). Alternatively or additionally, console 300 can operably attach to a stand-alone drive assembly 200, such as to provide electrical power, one or more control signals, and/or a motive force (e.g., via one or more linkages) to assembly 200. Console 300 can include processing unit 310, which can be configured to perform and/or facilitate one or more of the functions of system 10, such as one or more processes, energy deliveries (e.g., light and/or RF energy deliveries), data collections, data analyses, data transfers, data and/or signal processing, and/or other functions of system 10 (“functions of system 10” or “system functions” herein). Processing unit 310 can include processor 311, memory 312, and/or algorithm 315, each as shown. Memory 312 can be coupled to processor 311, and memory 312 can store instructions used by processor 311 to perform algorithm 315. Algorithm 315 can comprise one or more algorithms, such as one or more machine learning, neural net, and/or other artificial intelligence algorithms (“AI algorithm” herein). System 10 can include an interface, user interface 320, for providing and/or receiving information, to and/or from an operator of system 10. User interface 320 can be integrated into console 300 as shown. In some embodiments, user interface 320 comprises a component (e.g., a hand-held component) that is separate from a main portion (e.g., a console portion) of console 300, such as a display separate from, but operably attached to, the main portion of console 300. User interface 320 can include one, two, or more user input and/or user output components. For example, user interface 320 can comprise a joystick, keyboard, mouse, touchscreen, and/or another human interface device, user input device 321 shown. In some embodiments, user interface 320 comprises a display (e.g., a touchscreen display), such as display 322, also shown. In some embodiments, processor 311 can provide a graphical user interface, GUI 323, to be presented on and/or provided by display 322. User interface 320 can include an input and/or output device selected from the group consisting of: a speaker; an indicator light, such as an LED indicator; a haptic feedback device; a foot pedal; a switch such as a momentary switch; a microphone; a camera (e.g., when processor 311 enables eye tracking and/or other input via image processing of an image of an operator); and combinations of these.

In some embodiments, system 10 includes a data storage and processing device, server 400. Server 400 can comprise an “off-site” server (e.g., outside of the clinical site in which patient image data is recorded and/or other system 10 function is performed on a patient), such as a server owned, maintained, and/or otherwise provided by the manufacturer of system 10. Alternatively or additionally, server 400 can comprise a cloud-based server. Server 400 can include processing unit 410 shown, which can be configured to perform one or more functions of system 10, such as one or more system functions described herein. Processing unit 410 can include one or more algorithms, such as algorithm 315 described herein. Processing unit 410 can comprise a memory (not shown) that stores instructions such that a processing unit 410 can perform algorithm 315. Server 400 can be configured to receive and store various forms of data, such as: image data, diagnostic data, treatment planning data and/or treatment outcome data, data 420 shown and described herein. In some embodiments, data 420 can comprise data collected from multiple patients (e.g., multiple patients treated with system 10), such as data collected during and/or after clinical procedures performed on the multiple patients. In some embodiments, console 300 and server 400 are configured to communicate over a network, for example, a wide area network such as the Internet. Alternatively or additionally, system 10 can include a virtual private network (VPN), such as a VPN through which various devices of system 10 can transfer data. As described herein, the one or more functions of system 10 performed by processing unit 310 and/or 410 can be performed by either or both processing units.

In some embodiments, positioning assembly 130 includes one or more sensors, such as position sensor 1351 of articulation control assembly 135 shown. Position sensor 1351 can be configured to determine the shape, position, orientation, and/or location (“position” herein) of one or more portions of depositing device 100, such as one or more portions of positioning assembly 130 and/or infusion assembly 110. In some embodiments, position sensor 1351 operably attaches to console 300 and provides position information to processing unit 310. Algorithm 315 can analyze (also referred to as “process” herein) the position information to determine the position (e.g., the current anatomical location) of one or more portions of depositing device 100. Position information can be displayed to the operator of system 10 via GUI 323 of user interface 320, for example displaying an image comprising a rendering of position assembly 130 and/or infusion assembly 110 shown relative to the anatomy (e.g., an image of the anatomy of the patient and/or an exemplary anatomy image), such as relative to a portion of the GI tract and/or other internal organs of the patient (e.g., portion of the stomach and/or pancreas). In some embodiments, articulation control assembly 135 is operably attached to console 300, such that console 300 can provide movement commands for articulation control assembly 135 to robotically control and/or otherwise robotically manipulate positioning assembly 130 and/or infusion assembly 110. In some embodiments, console 300 provides movement commands based on operator input, such as one or more operator commands provided to console 300 via user input device 321. Additionally or alternatively, system 10 (e.g., via algorithm 315) can be configured to perform closed loop manipulation of depositing device 100, such as by analyzing data from position sensor 1351 and/or other sensors of system 10 and manipulating depositing device 100 based on the data analysis, to reach a desired location proximate a deposit site. In some embodiments, an operator can identify (e.g., and provide) a deposit site to system 10 (e.g., to be used by algorithm 315) via user input device 321, such that system 10 (e.g., via algorithm 315) can perform closed loop robotic manipulation of depositing device 100 to reach the identified deposit site.

In some embodiments, infusion assembly 110 includes an elongate tool, stylet 115. Stylet 115 can be removably positioned within lumen 112 of infusion assembly 110. In some embodiments, stylet 115 includes a lumen extending from its proximal end to its distal end. Stylet 115 can increase the stiffness of infusion assembly 110 when positioned within lumen 112. Stylet 115 can be removed from lumen 112 prior to the infusion of a material (e.g., treatment material 60) into the patient. In some embodiments, shaft 111 of infusion assembly 110 comprises a relatively flexible material, such as a soft plastic material, and stylet 115 comprises a relatively stiff material, for example a metal such as stainless steel. Stylet 115 can comprise a sharpened distal tip that allows for penetration through tissue, such as to a location proximate the deposit site (e.g., through the stomach wall and into the pancreas). In some embodiments, for example when shaft 111 comprises a flexible material and stylet 115 comprises a stiff material, stylet 115 and shaft 111 of infusion assembly 110 can be inserted into a location proximate a deposit site simultaneously. Once distal end 1118 of shaft 111 is properly positioned relative to the deposit site for an infusion, stylet 115 can be slidingly removed from lumen 112, such that only a flexible portion of shaft 111 remains inserted into the tissue. For infusions lasting extended periods (e.g., longer than one minute, such as approximately 10 minutes), this method of infusion via a flexible cannula (e.g., flexible shaft 111) can reduce the likelihood of injury to tissue caused by movement of depositing device 100 (e.g., movement caused by heavy patient breathing and/or unintentional operator movement of positioning assembly 130). In some embodiments, shaft 111 (e.g., a shaft 111 comprising a flexible material) can include one or more fenestrations (e.g., holes, slits, and/or other openings), such as fenestrations through which material 60 can pass through and into the target region of the patient. Shaft 111 can comprise one or more high density materials embedded within the walls of shaft 111, such as to provide hyper echogenicity to shaft 111.

In some embodiments, shaft 111 comprises a flexible needle (e.g., the entire length or just a distal portion of shaft 111 comprises a shaft with a needle-like construction), such as a needle with a gauge of no more than 27 G, such as no more than 28 G or 30 G. Additionally or alternatively, the distal portion of shaft 111 can comprise increased flexibility (e.g., more flexible than a more proximal portion of shaft 111), for example when the distal portion of shaft 111 comprises a smaller OD than a more proximal portion, and/or a smaller wall thickness. In some embodiments, at least a portion of shaft 111 (e.g., a distal portion of shaft 111) comprises one or more features configured to increase the flexibility of that portion of shaft 111, for example features comprising machine cut and/or laser etched rings in shaft 111 (e.g., that act as flexible joints). Shaft 111 (e.g., a needle) can comprise a coating (e.g., coating 1117 described herein, positioned on an inner or outer surface of shaft 111), such as a coating comprising a surfactant. In some embodiments, a functional element of depositing device 100 (e.g., functional element 199 described herein) comprises a coating (e.g., a biodegradable and/or otherwise degradable coating) applied to shaft 111 that increases the stiffness of shaft 111 (e.g., decrease the flexibility of shaft 111). The coating can be configured to rapidly degrade after the distal portion of shaft 111 is inserted into the pancreas (e.g., when the coating is exposed to a pH range of 8-9 within the pancreas), such that shaft 111 becomes more flexible after its insertion into the pancreas., such as to limit the risk of injury during an infusion of material 60 (e.g., an extended infusion, such as an infusion lasting at least 1 minute, such as up to 10 minutes). In some embodiments, a functional element 199 can comprise a stiffening sheath surrounding shaft 111 (e.g., a sheath that is manufactured over shaft 111 or a sheath that can be positioned over shaft 111 by an operator of system 10, the sheath configured to decrease the flexibility of shaft 111). The sheath can comprise a tear-away design constructed and arranged to be removed from shaft 111 (e.g., removed from proximal portion 1112 of shaft 111), such as a removal performed while shaft 111 is being inserted into the pancreas.

In some embodiments, treatment material 60 is provided (e.g., packaged by the manufacturer of system 10) to the operator within syringe assembly 120 (e.g., when syringe assembly 120 comprises a “pre-filled” syringe). Chamber 122 of a pre-filled syringe can comprise a volume of at least 1 ml, 2 ml, 5 ml or 10 ml (e.g., the largest prescribed and/or otherwise intended dose of treatment material 60), and/or no more than 2 ml, 5 ml, or 10 ml. In some embodiments, treatment material 60 is provided to the operator in a frozen state and is thawed prior to infusion via infusion assembly 110. Barrel 121 of pre-filled syringe assembly 120 can comprise a material configured to reduce the likelihood of treatment material 60 sticking to the walls of barrel 121, such as when barrel 121 comprises a glass and/or polypropylene material. Additionally or alternatively, barrel 121 can comprise a material with a color and/or other property configured to reduce UV exposure of treatment material 60, such as when barrel 121 comprises an amber color.

As described herein, depositing device 100 can be introduced into the patient via the patient's mouth. In some embodiments, system 10 includes one or more patient access devices, endoscope 80 shown. Endoscope 80 can comprise an endoscope, and/or another type of body access device (e.g., an introducer; sheath such as an endoscope-attached sheath; laparoscopic port; and/or other body access device). Endoscope 80 can include a handle, handle 81 shown, at its proximal end, and one or more working channels, working channel 82 shown, extending the length of the endoscope from the proximal to distal ends. Working channel 82 can slidingly receive a portion of depositing device 100, for example shaft 131 of positioning assembly 130 can be slidingly received within working channel 82 of endoscope 80 to introduce positioning assembly 130 into the patient (e.g., after the distal end of endoscope 80 has been positioned proximate a deposit site). In some embodiments, endoscope 80 and depositing device 100 are each introduced through the patient's mouth. In these embodiments, depositing device 100 can be introduced through working channel 82 and/or alongside endoscope 80.

In some embodiments, depositing device 100 comprises one or more visual and/or other indicators, marker 198 shown. Marker 198 can be located near the distal end of infusion assembly 110 and/or positioning assembly 130. Marker 198 can be located such as to indicate to the operator when the distal end of a first device is inserted a particular distance into a lumen of a second device. For example, when shaft 111 of infusion assembly 110 is being removed from lumen 132 of positioning assembly 130, marker 198 on shaft 111 can indicate to the operator when a pre-determined length (e.g., approximately five inches) of shaft 111 remains in lumen 132 (e.g., when marker 198 is located five inches from distal end 1118 of shaft 111). This configuration can indicate to the operator when the distal end of a device is proximate the proximal end of a lumen from which it is being removed, which can prevent (e.g., reduce) “whipping” of the distal end of the shaft and/or prevent (e.g., at least limit) unintentional discharge of treatment material 60. In some embodiments, marker 198 is located on distal portion 1317 of shaft 131, for example when shaft 131 is configured to be removed from working channel 82 of endoscope 80.

In some embodiments, system 10 is configured to deliver (e.g., via infusion assembly 110) one or more compounds, drugs, and/or other agents, agent 70 shown, to the patient, such as a delivery performed simultaneously and/or sequentially with delivery of treatment material 60, for example when agent 70 is delivered prior to, during, and/or after a delivery of treatment material 60. In some embodiments, agent 70 comprises one or more agents (e.g., hyaluronidase) configured to increase dissemination of material 60 in tissue. In some embodiments, agent 70 comprises one or more agents selected from the group consisting of: anti-inflammatory agent; immunosuppressant agent; pain blocking agent; nerve blocking agent; and combinations of these.

In some embodiments, agent 70 is delivered via infusion assembly 110 to a target site (e.g., a target region or other tissue location) prior to the delivery of treatment material 60. For example, an agent 70 comprising an adhesive, such as a low viscosity adhesive (e.g., glue), can be infused into an organ (e.g., the pancreas) prior to an infusion of treatment material 60. Agent 70 can travel from the infusion site throughout the interlobular space.

Agent 70 can comprise an adhesive that is configured to cure after a pre-determined period of time, for example at least 1 minute, and up to 10 minutes after infusion. Agent 70 can comprise an adhesive or other agent that is configured to hold the lobes of the pancreas together, allowing for a pressurized infusion of treatment material 60 while preventing or at least limiting treatment material 60 from “escaping” into the intralobular space of the pancreas. In some embodiments, agent 70 comprises a biodegradable and/or bioresorbable material (“biodegradable” or “bioresorbable” herein), for example agent 70 can be configured to biodegrade (e.g., to break down and be resorbed into the body) in a period of at least one day, and up to two weeks. In some embodiments, agent 70 comprises a synthetic hemostatic material, such as PuraStat. In some embodiments, agent 70 comprises a hydrogel, a hyaluronic acid-based compound, and/or any surgical sealant material.

In some embodiments, system 10 comprises one or more functional elements, such as functional element 99 shown. In some embodiments, depositing device 100 can comprise one or more functional elements, functional element 199 shown. Functional element 99 and/or functional element 199 (either or both, “functional element 99/199” herein) can comprise one or more sensors and/or one or more transducers, as described herein. In some embodiments, a functional element 99/199 can comprise a source of vacuum, a vacuum port, and/or any other vacuum-applying element, such as a vacuum-applying element configured to modify (e.g., enhance and/or otherwise modify) distribution of material 60 in tissue, and/or to stabilize device 100 in tissue (e.g., during delivery of material 60). In some embodiments, a functional element 99/199 can comprise a heat-applying element, a source of heat, and/or other heating element, such as a heating element configured to modify (e.g., enhance and/or otherwise modify) distribution of material 60 in tissue. In some embodiments, a functional element 99/199 can comprise a temperature-reducing element, a source of cooling, and/or other cooling element, such as a cooling element configured to modify (e.g., limit, stop, enhance, and/or otherwise modify) delivery of material 60 in tissue. In some embodiments, a functional element 99/199 can comprise an iontophoretic element, such as an iontophoretic element configured to modify (e.g., enhance and/or otherwise modify) delivery of material 60 in tissue.

In some embodiments, functional element 99/199 comprises an energy delivery device, for example a device configured to create an electric field in the tissue, such as an electric field that electroporates tissue. Reversable electroporation can enhance diffusion of treatment material 60 throughout the target region, for example when an electroporation electric field is created proximate the deposit site prior to, during, and/or after an infusion of treatment material 60. In some embodiments, functional element 99/199 comprises one, two, three, or more electrodes, such as one, two, three, or more electrodes of a functional element 199 that are located on a distal portion of shaft 111 and/or a functional element 99 comprising one or more “patch” electrodes that can be positioned on the skin of the patient. The electrodes can be configured to apply monopolar and/or bipolar RF energy to create an electric field (e.g., a pulsed electric field) configured to electroporate tissue. In some embodiments, the functional elements 99/199 create an electric field around the distal portion of shaft 111, such as around the distal one to two centimeters of shaft 111. In some embodiments, console 300 provides one or more drive signals to the one or more electrodes of functional element 99/199. Functional elements 99/199 can include one or more leads electrically connecting the electrodes to an energy delivery assembly of console 300. In some embodiments, the leads can comprise elongate conductors in which at least a portion of each lead is sputter coated along the length of shaft 111, such as when conductors are sputter coated on a thin film that is wrapped around and adhered to the surface of shaft 111. In some embodiments, electrodes of a functional element 99/199 can be integral to a flexible silicon chip that is adhered to the distal portion of shaft 111.

In some embodiments, functional element 99/199 comprises one, two, three, or more sound delivery elements (e.g., ultrasound transducers), such as one or more sound delivery elements configured to deliver sonoporation energy prior to, during, and/or after delivery of treatment material 60 to a target region of the patient.

In some embodiments, functional element 99/199 comprises a shape and/or position sensing element, such as navigational sensor 197 shown. Navigational sensor 197 can be configured to determine the position (e.g., the shape, orientation, location, and/or position) of at least a portion of itself and/or a device into which navigation sensor 197 is inserted. For example, navigational sensor 197 can be configured to record signals related to the position of sensor 197, and algorithm 315 can be configured to process (e.g., mathematically process, analyze, and the like) the recorded signals to determine the position of sensor 197. Navigational sensor 197 can comprise an elongate sensor (e.g., a “smart” stylet) that is slidingly received within a lumen of depositing device 100, such as within lumen 112 of infusion assembly 110 and/or lumen 132 of positioning assembly 130. Navigational sensor 197 can be operably connected to console 300, such as via a wired and/or wireless connection. Console 300 (e.g., via algorithm 315) can process position data from navigational sensor 197 along with other visual data (e.g., visual data recorded via an imaging device such as a functional element 99/199 comprising a camera) and/or other position data recorded by various sensors or functional elements of system 10. Algorithm 315 can be configured to process this recorded data to provide an operator with a real-time or near real time (“real time” herein) display of the position (e.g., the current anatomical location) of one or more devices of system 10 (e.g., the position of one or more portions of depositing device 100) relative to portions of the patient's anatomy (e.g., relative to the pancreas). Navigational sensor 197 can include an electromagnetic position sensor, such as a magnetic position sensor. Navigational sensor 197 can include a fiberoptic shape sensing sensor, such as a fiber Bragg sensor.

In some embodiments, system 10 is configured to perform a calibration procedure, such as a procedure used to calibrate one or more sensors of the system. For example, system 10 can enable an operator to register one or more imaging components (e.g., when functional element 99/199 comprises a visual camera) with one or more navigational components, such as navigational sensor 197. Console 300 can include image processing functions and/or navigation functions (e.g., one or more functions enabled by algorithm 315 and performed by processing unit 310) that provide image data and/or navigational data to an operator (e.g., via GUI 323 of user interface 320). The calibration procedure can be configured to register the data processed by these navigation and imaging processes. In some embodiments, the operator can align (e.g., manually align) the navigational components with depositing device 100 (e.g., with positioning assembly 130), for example when two components comprise alignment features, such as key and slot alignment features. In some embodiments, the operator can perform a calibration by inserting the navigational sensor 197 to known positions (e.g., depths) within a portion of depositing device 100 (e.g., by inserting to pre-determined depths within lumen 132 of positioning assembly 130), such as to insert to known positions indicated by one or more visual indicators located on navigational sensor 197. In some embodiments, visual indicators are located at regular intervals along navigational sensor 197, such as every centimeter along at least a portion of the length of navigational sensor 197. The operator can insert (e.g., advance into a lumen) navigational sensor 197 to a first location, record data related to the first location via console 300, and the operator can repeat the process at additional locations (e.g., depths) to determine the initial position of depositing device 100 and/or to calibrate various sensors of system 10, as described herein.

In some embodiments, lumen 132 of shaft 131 comprises two lumens, lumens 132a and 132b (e.g., two lumens in a side-by-side arrangement). Lumen 132a can be constructed and arranged to slidingly receive shaft 111 of infusion assembly 110. Lumen 132b can be constructed and arranged to slidingly receive navigational sensor 197. Shaft 131 can maintain the position of navigational sensor 197 relative to shaft 111 of infusion assembly 110. Shaft 131 can be introduced by an operator into the stomach of the patient, such as to position the distal portion of infusion assembly 110 proximate the pancreas. In some embodiments, navigational sensor 197 comprises a positional encoder configured to record data relating to the position of shaft 111 of infusion assembly 110 (e.g., based on recorded position data relating to positioning assembly 130). Console 300 can be configured to display to the operator, such as via GUI 323, a “projected” path of shaft 111 (e.g., the anatomical or other path that shaft 111 would follow if advanced from the distal end of lumen 132a of positioning assembly 130). Alternatively or additionally, shaft 111 of infusion assembly 110 and navigation sensor 197 are both slidingly received within a single lumen of positioning assembly 130, for example a single lumen 132.

In some embodiments, navigational sensor 197 comprises an elongate component that is slidingly received within lumen 112 of infusion assembly 110 (e.g., when navigational sensor 197 comprises an elongate body similar to an EUS stylet). Navigational sensor 197 can be configured to record position data related to infusion assembly 110. In some embodiments, console 300 (e.g., via algorithm 315) can analyze recorded position data to predict movement of infusion assembly 110 (e.g., distal end 1118 of shaft 111). In some embodiments, lumen 112 and navigational sensor 197 are constructed and arranged such that treatment material 60 can be infused through lumen 112 while navigational sensor 197 is inserted within lumen 112. In these embodiments, lumen 112 comprises an unobstructed cross-sectional area of at least 0.005 mm², 0.008 mm² and/or 0.010 mm² with navigational sensor 197 inserted, such that treatment material 60 can effectively pass through this unobstructed area.

In some embodiments, shaft 131 of positioning assembly 130 comprises a needle (e.g., the entire length or just a distal portion of shaft 131 comprises a needle or needle-like construction) that is configured to be advance into the stomach of the patient and through the gastric wall (e.g., through the wall of the stomach toward the pancreas). In some embodiments, shaft 131 is inserted through working channel 82 of endoscope 80, such as an endoscope which has been previously inserted into the stomach of the patient. Shaft 131 (e.g., a needle) can comprise a coating, such as a coating comprising a surfactant (e.g., positioned on an inner surface such as a luminal wall, and/or on an outer surface of shaft 131). Shaft 131 can comprise a needle (e.g., a shaft with a needle-like construction, at least on its distal portion) with a gauge of at least 19 G, such as at least 17 G. Lumen 132 of shaft 131 can slidingly receive both shaft 111 of infusion assembly 110 and navigation sensor 197. In some embodiments, shaft 111 can comprise a needle with a gauge of no more than 25 G, such as no more than 27 G, or 29 G. Shaft 111 and/or navigation sensor 197 can be extended from lumen 132 toward and/or into the pancreas together (e.g., at the same time) and/or independently (e.g., sequentially, at different times).

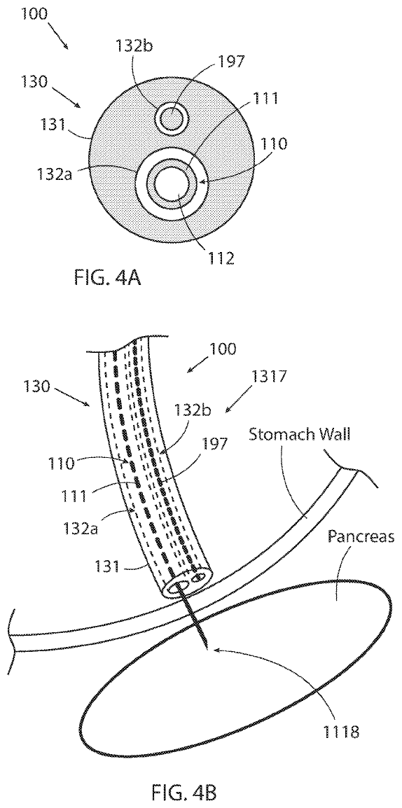

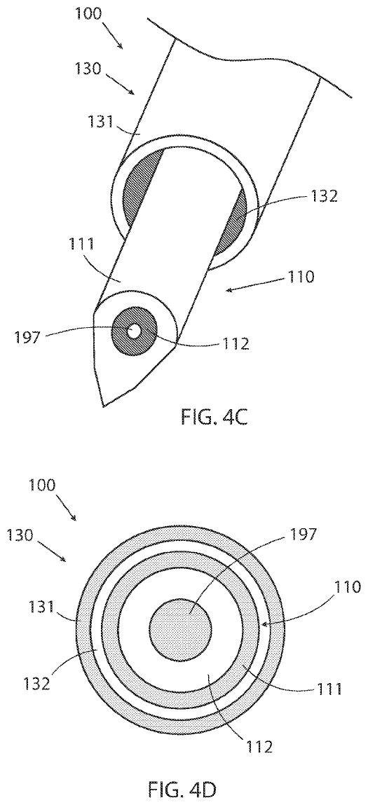

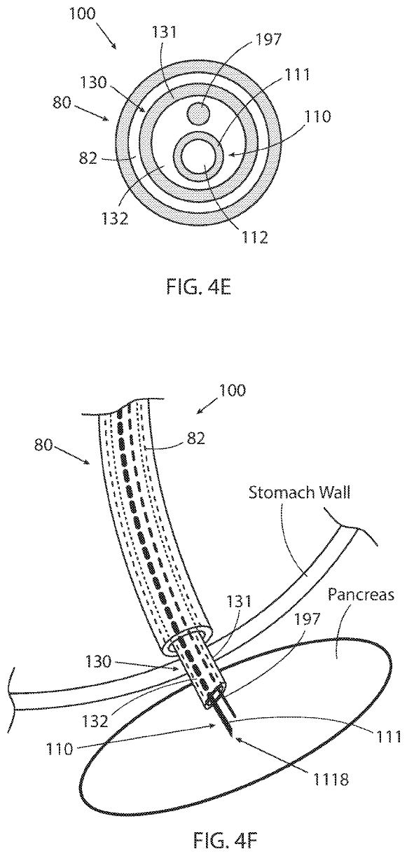

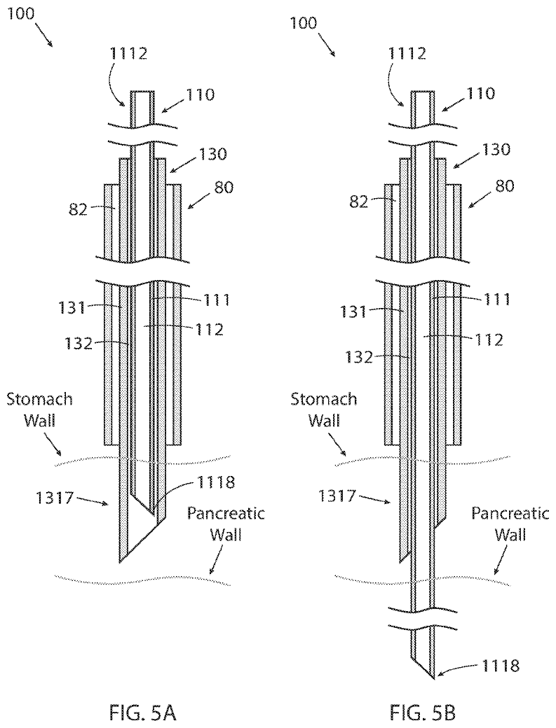

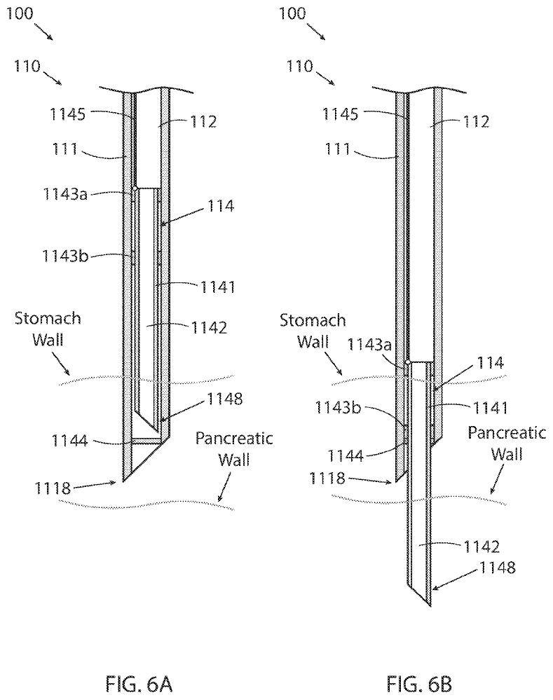

In some embodiments, the various arrangements of navigational sensor 197, positioning assembly 130, and infusion assembly 110 are constructed and arranged as described in reference to FIGS. 4A-4F herein. Other various arrangements of navigational sensor 197, positioning assembly 130, and infusion assembly 110 can be constructed and arranged as described in reference to FIGS. 5A-5B and/or FIGS. 6A-6B.

In some embodiments, navigational sensor 197 is located near distal end 1118 of shaft 111, such as within 1 cm, such as within 1.5 cm or 2 cm from distal end 1118. Console 300 can analyze signals from navigational sensor 197 to determine the location of distal end 1118 of shaft 111. In some embodiments, navigational sensor 197 comprises two position sensors, the second sensor located on shaft 111, at a location proximal to the first sensor, such as approximately 3 cm proximal to the first sensor. Console 300 can be configured to analyze signals from both navigational sensors 197 to determine a line in 2D and/or 3D space (e.g., the line on which the distal portion of shaft 111 is positioned in 2D and/or 3D space). Console 300 can display a “virtual needle” to the operator (e.g., via GUI 323), such as a virtual needle based on the line determined using the navigational data that can be overlaid on other image data, for example an overlay displayed on EUS image data. In some embodiments, shaft 111 comprises a needle that is difficult (e.g., too small) to sufficiently image using EUS imaging, such as a needle with a gauge of no more than 23 G, such as a gauge of no more than 25 G, 27 G, or 29 G. In these embodiments, navigational data recorded by navigational sensor 197 can be used by console 300 to display the position of shaft 111 to the operator when displaying EUS data, where the EUS image does not capture shaft 111. In some embodiments, navigational data recorded using navigational sensor 197 and EUS image data (e.g., data recorded by imaging device 90) can be used by system 10 to robotically control and/or otherwise robotically manipulate one or more infusions performed by system 10. In some embodiments, algorithm 315 can record the location of infusion assembly 110 (e.g., the location of distal end 1118) during each infusion of treatment material 60 and/or record the amount of treatment material 60 and/or other material deposited at each location (e.g., when multiple infusions are performed during a single clinical procedure).

In some embodiments, one or more of the various shafts of system 10 (e.g., shaft 111 and/or shaft 131) can comprise a material selected from the group consisting of: stainless steel, such as SS304 or SS316 stainless steel; nickel-titanium alloy; titanium; cobalt chromium; fused silica; polyethylene, such as HDPE or LDPE; polypropylene, poly ether ketone (PEEK); polytetrafluoroethylene (PTFE); fluoropolymers; polyether block amide (PEBA); sol-gel nanocomposites; and combinations of these. In some embodiments, one or more of the various lumens of system 10 (e.g., the walls of lumen 112, lumen 132, and/or working channel 82) can comprise hydrophobic and/or hydrophilic coatings. In some embodiments, one or more of the various lumens of system 10 (e.g., the walls of lumen 112, lumen 132, and/or working channel 82) can comprise a surfactant as a coating. In some embodiments, the shafts and/or other components described herein can comprise a material including co-formulations of materials, such as a polymer-based material co-formulated with one or more lubricious materials. In some embodiments, a shaft of system 10 (e.g., a shaft including a lumen extending therethrough) can comprise a single material, such as a stainless-steel shaft in which the wall of the shaft is stainless steel from the outer wall of the shaft to the inner wall of the lumen. Alternatively or additionally, a shaft of system 10 can comprise a laminate and/or other multi-part construction (e.g., a two-part construction), such as a shell and core construction, where a first portion, the shell, surrounds a second, inner portion, the core, where the lumen extends through the core. The shell and core of the shaft can comprise similar or dissimilar materials. In some embodiments, the core of a shaft comprises a more flexible material than the material of the shaft itself.

In some embodiments, system 10 includes one or more imaging devices, imaging device 90 shown, that can be configured to provide images of the patient and/or of one or more components of system 10 (e.g., images provided to an operator and/or used by an algorithm of system 10). In some embodiments, depositing device 100 can be configured to be inserted into the patient via an image-guided device (e.g., an endoscopic ultrasound device), for example when imaging device 90 and/or endoscope 80 comprise an image-guided device, such as an endoscopic ultrasound device (EUS). In some embodiments, depositing device 100, endoscope 80, and/or another component of system 10 comprises imaging device 90 (e.g., imaging device 90 is integral to that component). Imaging device 90 can comprise an imaging device selected from the group consisting of: a camera such as an endoscopic camera; an MRI imager; a CT imager; a fluoroscopic imager, an X-ray imager; an ultrasound imager such as an endoscopic or catheter-based ultrasound imager; and combinations of these. In some embodiments, treatment material 60 includes a radiopaque material and/or an ultrasonically reflective material, for example a material configured to be imaged by imaging device 90 when imaging device 90 comprises an X-ray or fluoroscopic imager, and/or an ultrasound imager, respectively. For example, treatment material 60 can comprise a gene therapy material (e.g., a gene therapy material in the form of a solution) including a chemical additive configured to be visible via an imaging device 90 (e.g., via X-ray imaging and/or ultrasound imaging modalities). Alternatively or additionally, treatment material 60 can comprise a dye or other visible material, such as to be visualized via a visible light camera. In some embodiments, the visualizable additive can be added to treatment material 60 during the manufacturing of treatment material 60 (e.g., at the time of packaging of treatment material 60), and/or in a clinical setting (e.g., by a clinician, nurse, and/or other operator of system 10), prior to treatment material 60 being deposited into the patient.

In some embodiments, imaging device 90 comprises an endoscopic ultrasound device configured to provide an endoscopic ultrasound (EUS) image (e.g., when endoscope 80 and/or depositing device 100 comprises imaging device 90). Alternatively or additionally, imaging device 90 comprises an ultrasound device and/or other imaging device configured to be positioned external to the patient's body during use (e.g., during imaging). In some embodiments, collected images (e.g., endoscopic ultrasound images) are analyzed (e.g., by algorithm 315 of system 10) and annotated (e.g., manually by an operator and/or automatically by system 10) to indicate (e.g., graphically indicate) one or more vessels (e.g., blood vessels) and/or other anatomical features of interest (e.g., related to the advancement of depositing device 100 and/or other component of system 10 advanced through internal locations of the patient), such as features selected from the group consisting of: arteries such as the splenic artery, veins such as the splenic vein; one or more ducts, such as the pancreatic duct; and combinations of these. Ultrasound images collected by system 10 (e.g., EUS images) can comprise multiple image types, such as standard ultrasound images and/or doppler ultrasound images. In some embodiments, collected ultrasound images (e.g., EUS images) comprise video. Analysis and annotation can be performed by system 10 in real-time (or near real-time, such as within 1 second of the image being captured, “real-time” herein). In some embodiments, system 10 enables an operator of system 10 to annotate one or more images, such as by using a pointing device (e.g., input device 321 comprising a computer mouse and/or a touch screen stylus) to identify and/or outline one or more anatomical features on a screen (e.g., display 322 of user interface 320 of console 300 or other display of system 10). Alternatively or additionally, an image analysis algorithm of system 10 (e.g., algorithm 315 described herein), such as a machine learning algorithm or other AI algorithm, automatically identifies and highlights these features on a screen. In some embodiments, system 10 enables the operator to correct and/or otherwise adjust the classification of any regions identified by the algorithm. In some embodiments, system 10 and/or the operator of system 10 identifies target areas to be treated in one or more collected images (e.g., an EUS image), such as images including one, two, or more target areas to be treated, prior to performing the treatment. System 10 can be configured to document (e.g., record and/or store) a treatment plan, such as a plan identifying particular regions to be treated. System 10 can be configured to document treatment plans for individual portions of a target area, such as the pancreas, such as by identifying one, two, or more deposits sites within the head, body, tail, and/or uncinate process of the pancreas. In some embodiments, system 10 (e.g., automatically by algorithm 315) and/or the operator (e.g., manually) can identify in the image (e.g., an EUS image) non-target areas to be avoided (e.g., avoided for delivery of treatment material 60 and/or avoided as a pathway of introduction of depositing device 100). In some embodiments, an image analysis is performed (e.g., automatically by algorithm 315 of system 10 and/or manually by an operator) to recognize and track infusion assembly 110 or another portion of device 100 (e.g., as it is advanced towards and/or into the tissue). In some embodiments, the operator is alerted (e.g., via a functional element configured as an audible, visual, and/or tactile alert) if depositing device 100 (e.g., infusion assembly 110) is advanced near, toward, and/or into a non-target region, and/or beyond a target region. In some embodiments, the operator is alerted if the projected path of infusion assembly 110 is not properly aimed at a deposit site. In some embodiments, system 10 is configured to automatically advance infusion assembly 110 to the deposit site, for example under motorized control of advancement. In some embodiments, system 10 is configured to prevent advancement (e.g., prevent automatic advancement) of infusion assembly 110 through and/or into an undesired volume of tissue (e.g., prevent advancement through and/or into a non-target region).

In some embodiments, imaging device 90 comprises a computed tomography (CT) device that is configured to provide a computed tomography (CT) image. In some embodiments, one or more CT images, and/or other images collected by system 10, are analyzed (e.g., by algorithm 315 of system 10) and annotated, such as to show one or more vessels or other anatomical features of interest, such as features selected from the group consisting of: arteries such as the splenic artery, veins such as the splenic vein; one or more ducts, such as the pancreatic duct; and combinations of these. In some embodiments, collected CT or other images can comprise a series of images such as a video. Analysis and annotation can be performed in real-time (or near real-time, such as within 1 second of the image being captured). In some embodiments, system 10 enables an operator of system 10 to annotate one or more images (e.g., manually or in a semi-automated arrangement), such as by using a pointing device (e.g., input device 321 comprising a computer mouse, a touch screen, and/or a touch screen stylus), such as to identify and/or outline one or more anatomical features on a display (e.g., display 322 of user interface 320). Alternatively or additionally, an image analysis algorithm of system 10 (e.g., algorithm 315 described herein), such as a machine learning algorithm or other AI algorithm, can automatically identify and note (e.g., highlight) these features on a display. In some embodiments, system 10 enables the operator to correct and/or otherwise adjust any regions identified by the algorithm (e.g., automatically identified by algorithm 315). In some embodiments, system 10 and/or the operator identifies target areas to be treated (e.g., identifies one or more deposit sites for depositing of treatment material 60 by depositing device 100) in an image (e.g., a CT image), such as when one, two, or more target areas to be treated (e.g., one, two, or more deposit sites) are identified (e.g., and noted in one or more images) prior to performing the treatment (e.g., depositing treatment material 60). System 10 can be configured to document a treatment plan, such as a plan identifying one or more regions to be treated (e.g., identifying one or more deposit sites in which treatment material 60 can be deposited by depositing device 100). System 10 can be configured to document (e.g., record and/or store) treatment plans for particular deposit sites, such as to document different treatment plans for different deposit sites, such as sites comprising individual portions of the pancreas (e.g., the head, body, tail, and/or uncinate process of the pancreas). In some embodiments, system 10 and/or the operator can identify in one or more images (e.g., CT images) one or more non-target areas in which treatment is to be avoided (e.g., treatment material 60 is not to be deposited and/or depositing device 100 is not to travel through). In some embodiments, an image (e.g., a CT image) is captured after infusion assembly 110 and/or other portion of device 100 is advanced towards and/or into the tissue of the particular deposit site, such as to include the portion of the device in the image to confirm that infusion assembly 110 and/or other portion of device 100 is in the right location. In some embodiments, an image analysis is performed to recognize and/or track infusion assembly 110 and/or other portion of device 100 as it is advanced towards and/or into the tissue. In some embodiments, system 10 is configured to alert the operator if infusion assembly 110 is advanced near and/or toward a non-target region and/or to a location beyond a deposit site. In some embodiments, system 10 is configured to alert the operator if the projected infusion assembly 110 path is not aimed at (e.g., not sufficiently directed toward) a deposit site. In some embodiments, system 10 is configured to automatically advance infusion assembly 110 to a deposit site, for example under motorized control of advancement (e.g., a robotic advancement). In some embodiments, system 10 is configured to prevent advancement (e.g., prevent automatic advancement) of infusion assembly 110 to a non-target region.

In some embodiments, algorithm 315 is configured to analyze image data (e.g., as described herein), where the image data comprises at least two different forms of image data, such as X-ray-based image data (e.g., fluoroscopic image data, CT image data, and/or other X-ray-based image data) and ultrasound-based image data (e.g., EUS-based image data). In these embodiments, analysis of the two forms of image data can be used to provide annotated image data feedback to an operator (e.g., as described herein), determine one or more paths of advancement of a system 10 device (e.g., as described herein), and/or perform another function.

In some embodiments, system 10 comprises an algorithm 315 that includes a bias, such as a bias toward or away from false-positives or false-negatives. For example, algorithm 315 can include a bias configured to cause a trajectory used to advance a component of system 10 (e.g., advance depositing device 100) toward and/or away from an object (e.g., toward or away from an organ or other anatomical location). Algorithm 315 can include a bias to tend to cause a planned trajectory and/or an actual trajectory of a depositing device 100 and/or a portion of depositing device 100 (e.g., the distal portion of device 100) to tend to travel away from particular tissue or other anatomical locations of a patient, such as to tend to cause the associated trajectory to tend to avoid certain tissue types and/or anatomical locations through which passage of device 100 is undesired.

In some embodiments, system 10 is configured to allow an operator to create registration data comprising data representing the location of device 100 on one or more images, such as images captured by imaging device 90 (e.g., images captured by an imaging device 90 comprising a single imaging device, or multiple images devices). The registration data can comprise sets of data representing various locations and/or orientations of device 100 in the patient in the performance of a treatment procedure. Registration data can be collected prior to a treatment procedure, at the beginning of a treatment procedure, and/or any time throughout the treatment procedure. The registration data collected can be used to align an image (e.g., a fluoroscopic or EUS image captured just prior to a treatment procedure) with a previously defined treatment plan (e.g., a treatment plan as described herein). For example, “pre-treatment image data” comprising a set of one or more images of the patient's anatomy can be created by a first imaging device 90 (e.g., a CT imager) at a time prior to a treatment procedure performed using system 10 (e.g., hours or days prior to a treatment procedure). A treatment plan can be created in which multiple (e.g., at least three, and/or up to ten) registration points are created in the pre-treatment image data (e.g., 2D and/or 3D data), the registration points representing various anatomical landmarks (e.g., branches of ducts or vessels) of the patient's anatomy. The treatment plan can also include registration points created in the pre-treatment image data that represent various locations to be treated using system 10. At the beginning of and/or during the treatment procedure, a second imaging device 90 (e.g., a fluoroscope and/or an EUS imager) can be used to create “treatment image data” representing additional images of the patient's anatomy that are created during and/or after a treatment procedure performed using system 10 (e.g., data 420 comprising treatment image data). System 10 (via algorithm 315) can integrate the registration data of the pre-treatment image data into the treatment image data (e.g., via a registration of the two sets of image data). System 10 can be further configured to provide a display of the treatment image data to an operator of system 10, such as when the registration points are shown as an overlay on the treatment image data. The display, including the overlay, can be used by an operator to guide the treatment. In some embodiments, system 10 is configured to automatically and/or semi-automatically (“automatically” herein) guide the treatment, such as by robotic control of device 100 based on the registration data (e.g., and continuously updated images of the patient's anatomy and device 100 as provided by second imaging device 90). In some embodiments, algorithm 315 is configured to provide command signals to a robotic control portion of system 10, such as when algorithm 315 is configured to advance the distal portion of device 100 through certain tissue locations while avoiding passing through other tissue locations (e.g., avoiding ducts, arteries, and/or veins as described herein). In some embodiments, algorithm 315 is configured to avoid certain tissue locations by a safety margin distance, such as a safety margin distance of at least 1 mm, at least 2 mm, or at least 4 mm.

System 10 can perform an automated and/or semi-automated procedure (an “automated” or a “robotic” procedure herein) comprising one or more automated infusions of treatment material 60 into an organ of the patient, such as the pancreas as described herein. Endoscope 80 can comprise an EUS endoscope where imaging device 90 is integrated into endoscope 80 and includes an ultrasonic imaging device. Navigational sensor 197 can be integrated into endoscope 80 and can be located proximate imaging device 90 (e.g., proximate an ultrasound imaging element), such as no more than 1.5 cm from imaging device 90, such as no more than 1.0 cm or 0.5 cm from imaging device 90. Alternatively or additionally, positioning assembly 130 can be slidingly received within working channel 82 of endoscope 80, and positioning assembly 130 can comprise navigational sensor 197 (e.g., navigational sensor 197 can be located on distal portion 1317 of shaft 131). The axial location of positioning assembly 130 can be registered (e.g., known) relative to endoscope 80. In some embodiments, imaging device 90 comprising an EUS imaging device can comprise a linear EUS imaging device and/or a radial EUS imaging device.

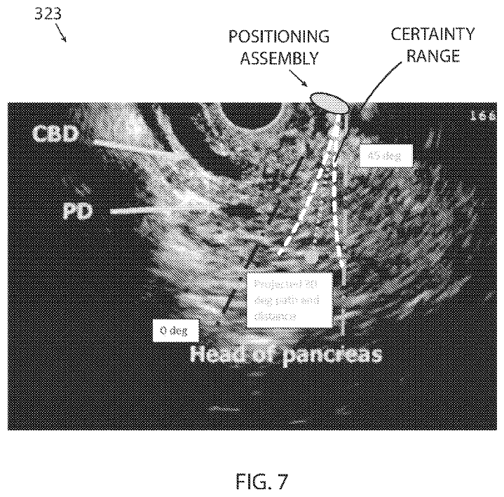

Algorithm 315 can record and analyze navigational data and/or image data (e.g., data 420 comprising navigational and/or image data) as endoscope 80 and/or depositing device 100 are inserted into the patient (e.g., inserted into the GI tract via the mouth of the patient) toward a deposit site. Algorithm 315 can process the data to form a 2D and/or 3D reconstruction of the patient anatomy. In some embodiments, algorithm 315 corrects and/or otherwise adjusts the recorded data for bodily motion, such as respiratory motion, cardiac motion, gastrointestinal peristalsis, and/or other bodily motion which may cause motion artifacts in the recorded data. For example, algorithm 315 can analyze differences in data collected over a short period of time, such as data collected over time periods of less than 0.3 s, such as less than 0.2 s or 0.1 s. In some embodiments, algorithm 315 identifies one or more bodily structures, such as one or more organs, vessels (e.g., blood vessels), and/or ducts (singly or collectively “anatomic features” herein) relative to a reconstruction (e.g., a 2D and/or 3D reconstruction) of the patient's anatomy. In some embodiments, algorithm 315 comprises one or more algorithms (e.g., one or more AI algorithms and/or other algorithms) configured to identify one or more anatomic features from the recorded data, such as by identifying features based on image data by 2D and/or 3D anatomy reconstructions based on navigational data. In some embodiments, algorithm 315 displays labels to the operator of various identified anatomic features identified by the algorithm (e.g., labels overlaid on EUS images and/or 2D and/or 3D reconstruction images displayed to the user on GUI 323). The location of labels overlaid on images displayed to the operator can be automatically updated by algorithm 315 as portions of the devices of system 10 are moved or otherwise repositioned within the patient. In some embodiments, algorithm 315 can “highlight” (e.g., overlay a visual indicator on a displayed image) one or more locations proximate a set of one or more deposit sites to the operator. Additionally or alternatively, algorithm 315 can highlight one or more locations of non-target tissue, such as ducts, blood vessels, and/or other tissue into which device 100 should not enter and/or into which material 60 should not be delivered. In some embodiments, system 10 (e.g., via algorithm 315) can alert and/or prevent the operator from performing an infusion into non-target areas. Algorithm 315 can display a virtual representation of one or more of the devices of system 10 relative to a 2D and/or 3D representation of the patient anatomy or other images generated by system 10, such as a virtual representation of at least a portion of infusion assembly 110, positioning assembly 130, and/or endoscope 80. Algorithm 315 can allow the operator to select (e.g., via input device 321) various 2D slice views of 3D data collected by system 10, such as a 2D view of a plane in which at least a portion of a device of system 10 is positioned. In some embodiments, system 10 (e.g., via algorithm 315) creates (e.g., and provides to an operator via user interface 320) one or more overlays of image data as described in reference to FIG. 7 herein.

In some embodiments, depositing device 100 (e.g., the distal portion of device 100) is constructed and arranged to be inserted into the pancreas or other location proximate a deposit site of the patient into which material (e.g., material 60) is to be delivered, such as when the insertion is performed via an ultrasound-guided percutaneous approach (e.g., access of one or more internal locations of the patient performed percutaneously), for example when shaft 111 of infusion assembly 110 comprises a needle configured to penetrate the skin of the patient, and imaging device 90 comprises an ultrasonic imaging device. Alternatively or additionally, depositing device 100 can be constructed and arranged to be inserted into the pancreas and/or other target region into which material (e.g., material 60) is to be delivered, the insertion performed via a CT-guided percutaneous approach, for example when shaft 111 of infusion assembly 110 comprises a needle configured to penetrate the skin of the patient, and imaging device 90 comprises a CT imaging device. In some embodiments, system 10 comprises a percutaneous introducer through which depositing device 100 can be advanced into the patient to a location such as the pancreas and/or other target region into which material (e.g., material 60) is to be delivered.

In some embodiments, system 10 can be configured (e.g., via algorithm 315) to perform pressure-controlled and/or flow-controlled infusions (e.g., of material 60), for example closed loop pressure-controlled and/or flow-controlled infusions, such as when depositing device 100 comprises a pressure sensor to monitor the pressure of material (e.g., material 60) within and/or exiting syringe assembly 120, and/or when drive assembly 200 is constructed and arranged to drive syringe assembly 120 in a manner to control the flow of material (e.g., material 60) from chamber 122, respectively.

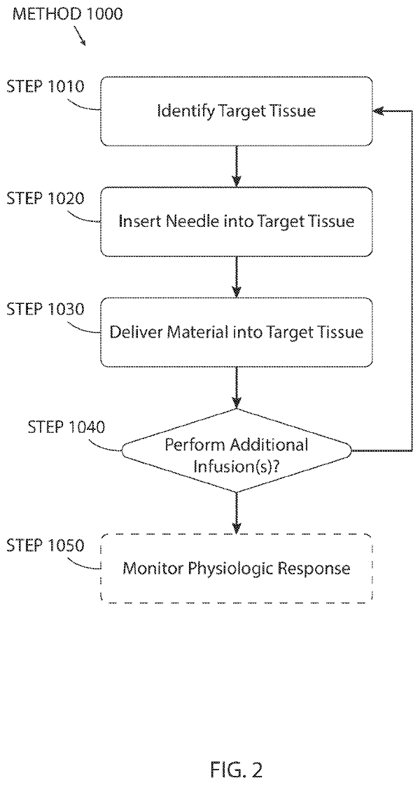

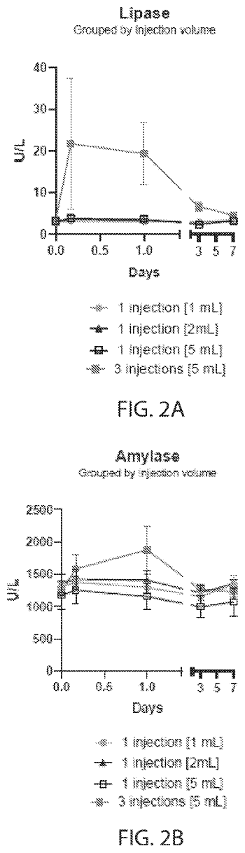

In some embodiments, system 10 is configured to perform one, two, three, or more infusions of a material, such as treatment material 60, into an organ of the patient, such as into the pancreas. In some embodiments, system 10 is configured to perform no more than 2, 3, 4, or 5 separate infusions into an organ in a single clinical procedure. Shaft 111 of infusion assembly 110 can comprise a needle of no more than 23 gauge, such as a needle of no more than 25 gauge. Each infusion of treatment material 60 by depositing device 100 can comprise an infusion of at least 1 mL of treatment material 60. Depositing device 100 can be constructed and arranged to perform each infusion with a controlled flow rate (e.g., a flow rate that does not exceed a maximum), such as a flow rate of no more than 20 mL/min, such as no more than 15 mL/min, 10 mL/min, 5 mL/min, 3 mL/min, and/or 1 mL/min, and/or a flow rate of at least 0.5 mL/min, 1 mL/min, 5 mL/min, 10 mL/min, and/or 25 mL/min. In some embodiments, infusions of treatment material 60 into the pancreas of the patient may cause transient elevations of pancreatic enzymes of no more than three times normal levels, such as no more than two times normal levels, where the elevated enzymes return to normal levels in a time period of no more than 72 hours, such as no more than 48 hours, or no more than 24 hours. In some embodiments, system 10 is configured to perform a set of infusions as described in reference to FIG. 2 herein.

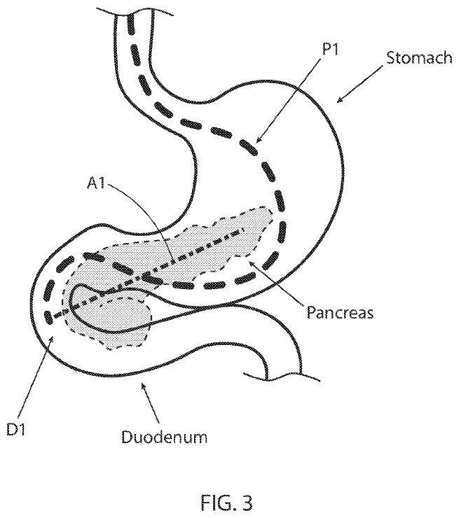

In some embodiments, system 10 is constructed and arranged to infuse treatment material 60 into the pancreas via the duodenum, such as via the duodenal D1 region. From this location, shaft 111 of infusion assembly 110 can penetrate the pancreas axially (e.g., along the long axis of the pancreas from the head to the tail), instead of orthogonally (e.g., when infusions are performed from the stomach). Shaft 111 can comprise a fenestrated needle (e.g., a needle including one or more holes, slits, and/or other openings in a side wall of the needle), such that treatment material 60 is diffusely dispersed via the fenestrations throughout the different parenchymal lobes of the pancreas through which shaft 111 is inserted (e.g., throughout the pancreatic head, neck, body, and/or tail in a single infusion step). A distal portion of shaft 111 can comprise one or more fenestrations through which treatment material 60 can be delivered. The fenestrations can be located along a portion of shaft 111 extending 100 mm to 200 mm from distal end 1118 of shaft 111. Axial insertion of infusion assembly 110 via the duodenal D1 region can be performed as described in reference to FIG. 3 and otherwise herein. In some embodiments, shaft 111 comprises fenestrations and an opening on the distal end (e.g., material 60 can exit shaft 111 via the fenestrations and the distal end of shaft 111).

As described herein, depositing device 100 and the other elongate devices of system 10 can be configured to be introduced into the body via a natural orifice (e.g., via the mouth or rectum), percutaneously, and/or via a skin incision (e.g., in an open surgical procedure or a minimally invasive surgical procedure). Depositing device 100 and the other elongate devices of system 10 can comprise one or more catheters, endoscopes, flexible probes, laparoscopic tools, syringes, and/or other flexible and/or rigid elongate devices. Device 100 can comprise a long (e.g., greater than 110 cm) flexible device (e.g., flexible probe or flexible syringe, “syringe” herein) that fits through a lumen, conduit, and/or other “working channel” of an access device (e.g., endoscope 80 described herein), such as a working channel of less than 4.2 mm diameter, such as less than 3.8 mm or less than 3.2 mm in diameter. Depositing device 100 can comprise a syringe that has at least one distal chamber configured to hold treatment material 60. In some embodiments, device 100 is configured to avoid waste of treatment material 60, such as by leaving undelivered portions of treatment material 60 within device 100 (e.g., not ejecting all of treatment material 60 from device 100), and/or to avoid requiring an additional fluid and/or an undesired volume of fluid (e.g., flush material) to clear treatment material 60 from device 100 (e.g., from one or more lumens of device 100). In some embodiments, device 100 comprises a plunger (e.g., plunger 124 described herein) configured to deliver (e.g., force or otherwise deliver) treatment material 60 from the device 100. Device 100 can be configured to be inserted into the patient under fluoroscopic guidance. In some embodiments, device 100 comprises one or more radiopaque markers (e.g., marker 198) and/or a radiopaque shaft such that it can be visualized fluoroscopically. Device 100 can include at least two lumens (e.g., as described herein), for example at least one lumen configured to slidingly receive a guidewire and/or to deliver contrast material. In some embodiments, device 100 can comprise a configuration of two lumens in a shape approximating a smile (“smiley lumens”), two circular lumens, and/or two D-shaped lumens. Device 100 can comprise one or more lumens configured for: receiving a vacuum, such as when configured as a desufflation lumen; delivering a contrast material; depositing or otherwise delivering treatment material 60; receiving a guidewire; enabling pressure monitoring within the patient (e.g., by fluidly connecting an area within the patient to a pressure monitor outside the patient); enabling steering of device 100, such as by receiving a steering cable; and combinations of these. Device 100 can be configured to be delivered over a guidewire. Device 100 can include an occluding mechanism (not shown, but such as a functional element 199 comprising a balloon) that is positioned around a distal portion of device 100 and configured to prevent or at least reduce backflow of treatment material 60 (e.g., prevent backflow of treatment material 60 out of the pancreas). Alternatively or additionally, a mechanism of device 100 (e.g., a functional element 199 configured as a vacuum port) can be configured to apply suction to a tissue wall, such as to draw the shaft of device 100 toward the tissue wall. Device 100 can be steerable in at least one direction. Device 100 can have a torque transfer mechanism (e.g., a functional element 199 configured as a torque transfer mechanism) that can be configured to change the direction of steering in response to a torsional force (e.g., a force provided by a torque cable). Device 100 can have variable stiffness along its length (e.g., comprise a shaft with a stiffer proximal portion than distal portion). Device 100 can include a chamber (e.g., not shown, such as a functional element 199 comprising a chamber in a distal tip or other distal portion of device 100) that is sized and arranged to allow insertion of a container that contains treatment material 60 (e.g., a container that is also used to transport treatment material 60), such as to simplify the process of loading device 100 with treatment material 60. Treatment material 60 can be loaded into device 100 in a manner similar to loading a conventional syringe, where the plunger is first fully depressed, then the tip of device 100 is placed into a container with the treatment material 60 so that a lumen is in contact with treatment material 60, after which the plunger is withdrawn to draw treatment material 60 into device 100. The proximal end of plunger 124 can be configured to be depressed manually, and/or it can be configured to be controlled by a component (e.g., a motor, spring-activated mechanism, and/or pneumatic or hydraulic mechanism of system 10 such as via algorithm 315) that provides linear force in at least a forward direction, such as both a forward direction and a backward direction. The proximal end of device 100 can be configured to be manually steered and/or it can be configured to be robotically steered. The proximal end of device 100 can be configured to measure the pressure of fluid in the device 100 and/or the pressure proximate a deposit site (e.g., when the deposit site comprises a duct or natural body lumen), such as via a sensor-based functional element 99/199. The proximal end of device 100 can be configured to control pressure of the fluid within a deposit site (e.g., within a duct, for example by controlling the syringe distal-facing element). Additionally or alternatively, device 100 can be configured to monitor the pressure within the deposit site, for example via a device lumen and/or by depositing a known volume of treatment material 60 into the deposit site. In some embodiments, system 10 is configured to measure, monitor, control, and/or limit the pressure in which treatment material 60 is delivered to a deposit site. For example, system 10 can be configured to measure and/or control the pressure within the tissue into which treatment material 60 is delivered, such as to maintain a pressure within the tissue below a maximum of 8 mmHg, such as a maximum of no more than 5 mmHg, 3 mmHg, or 0 mmHg. In order to prevent this pressure maximum from being exceeded, system 10 can, via a sensor-based functional element 99/199, measure, monitor and/or control a pressure within one or more components of system 10, such as within a syringe (e.g., of syringe assembly 120) and/or within a depositing device 100, such as within a fluid pathway of a syringe (e.g., syringe assembly 120) and/or a device 100, whereby that measured, monitored, and/or controlled pressure can be correlated to the pressure of treatment material 60 within the tissue (e.g., within the deposit site). In some embodiments, a second syringe (e.g., the same or different syringe assembly 120) can be attached to a second fluid lumen of device 100, such as a lumen configured to deliver an imaging agent, a diluent, a viscous fluid, and/or a fluid that becomes a gel within the body. The second syringe can be controlled manually and/or automatically (e.g., via processing algorithm 315). In these embodiments, the first syringe can be configured to control the volume of treatment material 60 delivered to the patient, while the second syringe can be used to manage the pressure of the delivered materials. In some embodiments, delivery of treatment material 60 can be performed at an elevated pressure, such as to improve contact of treatment material 60 (e.g., a virus) with the intended treatment site. For example, system 10 can be configured to measure and/or control the pressure within the tissue into which treatment material 60 is delivered, and to maintain the pressure within the tissue above a minimum, such as a minimum of at least 8 mmHg, 14 mmHg, and/or 20 mmHg, such as to enable opening of interstitial fluid pathways to engender flow of treatment material 60 through the tissue (e.g., through the deposit site). In some embodiments, system 10 includes a functional element 99/199 comprising a pressure sensor, a flow rate monitor, and/or volume sensor that produces a signal that is used by system 10 (e.g., by an algorithm 315) to regulate the amount, rate, and/or pressure of injectate (e.g., treatment material 60) within device 100 and/or within the pancreas and/or other deposit site (e.g., above a minimum pressure and/or below a maximum pressure). Alternatively or additionally, system 10 can include a sensor (e.g., a sensor-based functional element 199 and/or other functional element of system 10) such as a pressure sensor or impedance sensor to determine whether device 100 is in contact with a desired tissue location.

In some embodiments, shaft 111 of infusion assembly 110 comprises a needle or other elongate structure that includes a varying outer diameter, such as to prevent or at least limit the likelihood of shaft 111 from being inserted deeper into tissue than desired. In some embodiments, shaft 111 comprises one or more portions with a sloped cross-sectional profile, such as a continuously increasing diameter in the one or more portions. Alternatively or additionally, shaft 111 can comprise a needle or other elongate structure that comprises a diameter that changes in magnitude in one or more discrete steps, a “stepped” profile. In some embodiments, shaft 111 can comprise a stepped profile, a sloped profile, or both, that is configured to prevent a puncture into a tissue surface (e.g., prevent a puncture through the surface of the stomach wall and into the pancreas) beyond 4 cm, such as to prevent a puncture beyond 3 cm, 2 cm, or 1 cm. For example, the position of a single stepped portion of shaft 111 can be based on a minimum stomach wall thickness and a minimum depth of the main pancreatic duct, such that shaft 111 doesn't penetrate beyond the shallowest expected depth of the main pancreatic duct (e.g., when shaft 111 is inserted into the pancreas via the stomach). In some embodiments, a stepped portion (e.g., a most distal, smaller diameter stepped portion) of shaft 111 comprises a diameter of 27 g, 28 g, 29 g, 30 g, or 33 g. The distal portion (e.g., the distal stepped portion) of shaft 111 can include a coating for enhanced visualization (e.g., enhanced visualization under ultrasound imaging and/or fluoroscopic imaging). Alternatively or additionally, the distal portion of shaft 111 can include a modification for enhanced visualization, such as a modification created via sand blasting and/or laser etching. In some embodiments, the portion of shaft 111 just proximal to the most distal stepped portion comprises a diameter of 27 g, 25 g, or 23 g (e.g., larger than the diameter of the most distal portion, thus creating a step to limit travel). In some embodiments, shaft 111 comprises an atraumatic distal tip (e.g., shaft 111 comprises an unsharpened, relatively blunt, and/or otherwise atraumatic tip). In some embodiments, shaft 111 comprises a sharpened tip, such as an offset beveled tip or a central point beveled tip. In some embodiments, a proximal portion of shaft 111 (e.g., proximal portion 1112) comprises a stiffness that is greater than a distal portion of shaft 111, such as to increase the pushability of shaft 111 (e.g., pushability through lumen 132 of positioning assembly 130). In some embodiments, the length of one or more portions of the stepped profile can be adjusted (e.g., adjusted by an operator of system 10), for example when shaft 111 comprises one or more telescoping portions, such as locking, telescoping portions that are operator adjustable prior to and/or after shaft 111 has been introduced into the patient.

Applicant has performed non-human clinical studies to evaluate the safety and efficacy of the systems and methods of treatment described herein. Applicant has performed studies testing the safety and efficacy of the EUS-guided procedure and automated delivery system, as described herein, for delivering a material 60 comprising targeted gene therapy to a patient. Metabolic diseases such as Type 2 diabetes and obesity may benefit from local therapeutic delivery directly into the pancreas, such as to improve on-target efficacy, safety, and durability while limiting off-target safety risks. Advancements in adeno-associated virus (AAV)-based technologies have expanded the ability of gene therapy as a one-time treatment strategy for metabolic diseases. Applicant has developed a novel AAV-based gene therapy platform and endoscopic ultrasound (EUS)-guided automated delivery system that enables local and durable production of therapeutic proteins by the pancreas. The aim of some of the studies was to assess the feasibility and safety of infusing AAV into the pancreas using a system 10 comprising an EUS-guided automated delivery system in a Yucatan pig model.

An endoscope (e.g., endoscope 80) with a 25-gauge needle (e.g., shaft 111 of infusion assembly 110) integrated with a fluid delivery system (e.g., depositing device 100) is tracked into the porcine stomach. Utilizing an ultrasound view, anatomic landmarks such as the splenic artery, spleen, and liver are used to guide infusions into the parenchymal tissue of the targeted splenic lobe of the porcine pancreas equivalent to the human body and tail of the pancreas. One or three infusion sites are typically evaluated per procedure.

Toluidine blue (TBO) dye infusion is used to optimize procedural parameters including number of infusions, flow rate, and fluid pressure to establish initial procedure efficacy and minimize safety risk prior to AAV infusion. Study results included biopsies from different splenic lobes were qualitatively scored from 0 to 5 based on dye saturation with 0=no dye and 5=completely dyed (n=14 biopsies per animal [n=4, connecting lobe; n=2, duodenal lobe; n=8 splenic lobe]). TBO dye was determined to be extensively distributed throughout the splenic lobe of the pancreas with either one or three infusions as evidenced by dye score. There was minimal dye distribution in untargeted regions of the pancreas and other tissues.

An expression cassette containing a promoter in green fluorescent protein (GFP) reporter transgene was packed into AAV vectors (AAV-GFP) and infused into the pancreas at one to three sites using four escalating doses to establish dose response and proof-of-concept viral vector biodistribution in the targeted splenic lobe. Vehicle (saline) was used as a control (N=13).

Similar to TBO dye, AAV-GFP Infusion using system 10 demonstrated a localized biodistribution in the pancreas with activity (e.g., robust activity) in up to 80% of cells in the targeted splenic lobe as determined by histological GFP expression at three weeks post procedure (62+/−10% SEM, N=4, dose=1.5e14 vector genome [VG]). Histological analysis of the untargeted duodenal and connecting lobes showed minimal GFP expression. In some embodiments of the present inventive concepts, system 10 is configured to generate a localized diffuse distribution of treatment material 60 in a targeted region of the pancreas to at least 5% of cells in the targeted region (e.g., the targeted splenic lobe), such as a diffuse distribution to at least 5%, 10%, 15%, 20%, 30%, 40%, 50%, 60%, 70% of cells in the targeted region (e.g., the targeted splenic lobe). This activity level can be achieved with a number of infusions that does not exceed 5 infusions (i.e. does not exceed more than 5 separate deliveries of treatment material 60), such as no more than 3 infusions (i.e. does not exceed more than 3 separate deliveries of treatment material 60), and the treatments can avoid either or both of pancreatitis and/or lipase elevation above three times the upper limit of normal (3×ULN). This activity level can be achieved with a risk of pancreatitis that is no more than 1.0%, no more than 0.5%, and/or no more than 0.1%.

In applicant's studies, serum alanine transaminase (ALT) was evaluated as an initial measure of systemic safety and liver health. ALT remained in the normal ranges across all points measured. Serum lipase was assessed as an indicator of pancreatitis and remained in the normal range across most time points evaluated. In one AAV-GFP treated animal, transient elevations in serum lipase were detected at 4 hours, peaked at 24 hours (~2.5×above baseline), and resolved by 72 hours post-procedure. At four weeks post-procedure, high levels of pancreatic GFP expression significantly correlated with elevated lipase, which was attributed to durable expression and immunogenicity of the foreign GFP reporter.

These studies performed by applicant provide proof of concept that an EUS-guided procedure and automated delivery system can effectively deliver gene therapy into the pancreas while mitigating local and systemic safety concerns. A local gene therapy approach may provide a new platform for one time treatment of metabolic diseases, including Type 2 diabetes and obesity.

Applicant has also performed non-human clinical studies in a mouse model to test the safety and efficacy of an EUS-guided procedure using system 10 for delivering targeted gene therapy to a patient. In Type 2 diabetes (T2D), beta-cell (B-cell) dysfunction and failure are early drivers of declining glucose tolerance and disease progression. GLP-1 receptor antagonists (GLP1rAs) improve β-cell function and have proven clinical efficacy, but their utility is limited by half-life constraints and systemic side effects. Applicant has developed an AAV-based gene therapy platform and endoscopic ultrasound (EUS)-guided automated delivery system (system 10) that enables local and durable production of therapeutic proteins by the pancreas toward improving islet function. To address the limitations of current GLP1RA therapies, in vivo screening capabilities were utilized in applicant's studies to identify AAV-GLP1RA gene therapy candidates optimized for driving local β-cell production of GLP1RA and subsequent improvements in islet function.

DNA plasmids encoding unique GLP1RA constructs optimized for the generation of functional GLP1RA protein are screened for enhanced GLP1RA production and secretion from a mouse insulinoma β-cell (MIN6) line, followed by a cyclic adenosine monophosphate (cAMP)-based Chinese hamster ovary (CHO) GLP-1 receptor (GLP1R) reporter assay to confirm the functional activity of secreted GLP1RA protein. A gene expression cassette was developed with an optimized GLP1RA transgene and promoter sequence to restrict expression to β-cells and packaged into AAV vectors to create AAV-GLP1RA. Primary db/db mouse islets, human EndoC-BH5 β-cells, and human islets were transfected with AAV-GLP1RA to assess gene therapy driven β-cell and islet functionality.

MIN6 murine β-cell screening of over 100 GLP1RA transgene candidates demonstrated robust glucose-simulated GLP1RA production and secretion. Secreted GLP1RA protein concentrations of 150-450 pM (gray area) were detectable with optimized candidates (mean +/−SEM, N=3-16 per candidate) compared to preproglucagon (GCG) positive control.

A subset of super supernatants from transfected MIN6 cells were evaluated to confirm functional GLPIR signaling using a human GLPIR CHO cAMP reporter cell line. Analyses revealed a 1.3-1.6-fold increase in cAMP Signaling compared to GCG positive control (mean +/−SD, p<0.05, N=3-4 per candidate supernatant), indicating that top transgene-induced secreted GLP1RA proteins were functional and capable of activating the GLP1R.

Primary islets isolated from 8-week-old db/db T2D mice were transduced with AAV-GLP1RA to assess transgene-driven GLP1RA protein production and glucose stimulated insulin secretion (GSIS) in diabetic islets. Fluorescence microscopy analysis determined that islets can be effectively transduced by AAV with green fluorescent protein (GFP) reporter expression in 30% of cells. AAV-GLP1RA induced islet GLP1RA protein secretion in a dose-dependent manner (mean +/−SEM, p<0.001, N=3-8 per group). High dose AAV-GLP1RA improved GSIS as evidenced by a 2.2-fold increase in stimulation index (GSIS/basal insulin secretion) without 3-isobutyl-1-methylzanthine (IBMX, cAMP enhancer) compared to untransduced controls (mean +/−SEM, p<0.0001, N=3-8 per group), highlighting that AAV-GLP1RA can improve β-cell function in mouse diabetic islets.

EndoC-BH5 β-cells were transduced with AAV-GLP1RA to evaluate the impact of transgene-driven effects in GLP1 responsive human-cells. GLP1RA protein was produced and secreted in a dose-dependent manner following AAV-GLP1RA transduction (mean +/−SD, p<0.0001, N=2-3 per group). A 2.1-2.5-fold increase relative to untransduced controls (mean +/−SD, p<0.05, N=2-3 per group) was observed. This effect was abrogated by a GLP1R antagonist, Exendin-9 (Ex9), underscoring the ability of AAV-GLP1RA to improve human β-cell function in a GLPIR dependent manner.

Islets were isolated from a healthy, overweight, non-T2D donor and transduced with high and low doses of AAV-GLP1RA to evaluate β-cell transduction efficacy and GSIS. AAV-GLP1RA transduced 10%-30% of β-cells in a dose dependent manner as evidenced by GFP and NKX6.1 (β-cell specific marker) immunostaining colocalization (p<0.001, mean +/−SEM, N=9-10 per group). High-dose AAV-GLP1RA improved GSIS, as evidenced by a significant increase in stimulation index compared to untransduced controls (p<0.01, mean +/−SEM, N=10-11 per group), indicating that AAV-GLP1RA is capable of improving B-cell function in primary human islets.

These study analyses demonstrate the ability of applicant's screening capabilities for identifying optimized, β-cell-targeted, gene therapy candidates capable of improving islet function in vitro. Localized GLP1RA-based gene therapy has the potential to durably restore β-cell function, delay disease progression, and reduce the therapeutic burden for T2D patients.

Referring now to FIG. 2, a method of treating a patient while limiting transient physiologic response is illustrated, consistent with the present inventive concepts. FIG. 2 illustrates Method 1000 for infusing the pancreas with a material (e.g., treatment material 60), where the physiologic response caused by the infusion process returns to normal within an expected timeframe. The steps of Method 1000 can be performed with the various devices and components of system 10 described in reference to FIG. 1 and otherwise herein. In Step 1010 target tissue (e.g., target tissue within a target region, as described herein) is identified, for example the parenchyma of the pancreas of the patient. In some embodiments, target tissue can be identified using endoscopic ultrasound (EUS), for example when endoscope 80 has been inserted through the mouth of the patient into the stomach, proximate the pancreas. In some embodiments, target tissue can be located in one, two, or more target regions (regions to receive material 60 that has been delivered into one or more deposit sites), such as one, two, or more regions of the head, neck, body, and/or tail of the pancreas (e.g., target tissue to be treated can comprise tissue in both the body region and tail region of the pancreas). In some embodiments, a target region comprises a region of the pancreas containing malignant and/or cancerous tissue. In some embodiments, a target region comprises target tissue comprising target types of cells, such as malignant or cancerous cells. In some embodiments, target types of cells comprise cell types selected from the group consisting of: a type of exocrine cell such as acinar cells, duct cells or other exocrine cells; a type of endocrine cell such as alpha cells, beta cells, delta cells, epsilon cells, pp cells, G cells, or other endocrine cells; a type of vascular cell, such as endothelial cells, pericytes, smooth muscle cells, or other vascular cells; a type of immune cell, such as macrophages, mast cells, T cells, B cells, lymphocytes, dendritic cells, or other types of immune cells; and combinations of these In Step 1020, shaft 111 of infusion assembly 110 is inserted to an anatomical location proximate a deposit site, such that treatment material 60 can be infused through lumen 112 of shaft 111 and into a target region (e.g., via one or more deposit sites). In some embodiments, distal end 1118 of shaft 111 can be positioned proximate a deposit site using positioning assembly 130. After positioning, shaft 111 can be advanced from lumen 132 of positioning assembly 130 and into the location proximate the deposit site. In Step 1030, an infusion is performed, where treatment material 60 is delivered from syringe assembly 120, through infusion assembly 110 and into the target region via the deposit site. In some embodiments, the infusion is performed at a flow rate of no more than 20 mL/min, 15 mL/min, 10 mL/min, 5 mL/min, 3 mL/min, and/or 1 mL/min, and/or a flow rate of at least 0.5 mL/min, 1 mL/min, 5 mL/min, 10 mL/min, and/or 25 mL/min.

In some embodiments, method 1000 can be performed to treat target tissue representing a particular percentage of the overall pancreatic volume (e.g., a minimum, maximum, and/or target percentage of the parenchyma tissue of the pancreas), such as a minimum of at least 20% of the overall pancreatic volume. In some embodiments, treatment material 60 is infused into at least 10% of a target region, such as into at least 20%, at least 30%, at least 40%, or at least 50% of the volume of the target region. Alternatively or additionally, treatment material 60 can be infused into no more than 30% of a target region, such as into no more than 40% or 50% of the volume of the target region. System 10 can be constructed and arranged to avoid infusion of treatment material 60 to non-target regions (e.g., into non-target tissue), such as non-target regions of the pancreas (e.g., identified prior to and/or during the infusion procedure). System 10 can be configured to limit systemic distribution of the treatment material 60, such as to limit the systemic distribution to a level that is below a toxic level to one or more non-pancreatic organs. Alternatively or additionally, system 10 can be configured to provide systemic distribution (e.g., a non-zero systemic distribution) of treatment material 60, such as to cause a systemic distribution of the treatment material at a level that provides a therapeutic dose to one or more non-pancreatic organs.

In Step 1040, if additional infusions are to be performed, the method returns to Step 1010. In some embodiments, Step 1010 through Step 1030 are repeated such that two, three, or more infusions are performed into different portions of target tissue (e.g., at different deposit sites located at different portions of the pancreas). Alternatively or additionally, Step 1010 through Step 1030 are repeated no more than 5 times, such as no more than 4, no more than 3, or no more than 2 times.

In Step 1040, if no more infusions are to be performed, in an optional Step 1050, system 10 can monitor physiologic responses by the patient that may be caused by the infusions. For example, the patient may experience transient elevations in pancreatic enzymes, for example elevations in amylase and/or lipase. Applicant has performed studies using system 10, the results of which have shown transient elevations remain below three times the upper bound of normal levels, such as below two times the upper bound of normal levels. Following the treatment of method 1000 on a pancreas, elevations in pancreatic enzymes are expected to return to baseline levels. Pancreatic studies performed by the applicant using system 10 have shown that pancreatic enzymes return to normal levels within 72 hours, such as within 48 hours, or within 24 hours. Study results are described herebelow in reference to FIGS. 2A and 2B. In some embodiments, method 1000 using system 10 results in acute pancreatitis in patients (e.g., human patients) in less than 3% of procedures performed, such as less than 2%, less than 1%, less than 0.5%, and/or less than 0.1% of the procedures performed.

Referring additionally to FIGS. 2A and 2B, graphs of pancreatic enzyme levels monitored during a non-human animal study performed by the applicant are illustrated, consistent with the present inventive concepts. FIG. 2A illustrates the recorded response of lipase enzymes in reaction to various infusion volumes and number of infusions performed. FIG. 2B illustrates the recorded response of amylase enzymes in reaction to various infusion volumes and number of infusions performed. As shown in the graphs, enzyme levels remain below a threshold associated with pancreatitis (e.g., three times the upper limit of normal), and these levels resolve within 48 to 72 hours.

Referring now to FIG. 3, an anatomic view of a portion of a patient's GI tract and pancreas is illustrated, consistent with the present inventive concepts. Depositing device 100 and other devices of FIG. 3 can be of similar construction and arrangement to the various devices of system 10 described in reference to FIG. 1 and otherwise herein. In some embodiments, depositing device 100 (not shown) can be inserted, via the patient's mouth, through the stomach and into the proximal duodenum, such as along path P1 shown. The distal portion of depositing device 100 can be positioned proximate the head of the pancreas within region D1 of the duodenum. Infusion assembly 110 can be inserted axially, along axis A1 shown, following a delivery pathway through the duodenal wall and into the pancreas to a deposit site, the pathway extending from the head to the tail of the pancreas. In some embodiments, shaft 111 of infusion assembly 110 comprises one or more fenestrations along the length of shaft 111, such that a diffuse infusion of material 60 can be performed (e.g., as described herein) along the length of the pancreas (e.g., through the fenestrations along axis Al shown). Alternatively or additionally, depositing device 100 can be constructed and arranged to perform one or more infusions into the pancreas via a delivery pathway of infusion assembly 110 through the stomach (e.g., through the stomach wall, and the pancreas orthogonal to axis A1), such as is described herein.

Referring now to FIGS. 4A-4F, various sectional and perspective views of a depositing device are illustrated, consistent with the present inventive concepts. Depositing device 100 and other components of FIGS. 4A-4F can be of similar construction and arrangement to similar components described in reference to FIG. 1 and otherwise herein. FIG. 4A illustrates a sectional view of a portion of depositing device 100, where the illustrated section is orthogonal to the long axis of shaft 131 of positioning assembly 130 (e.g., a section through distal portion 1317 of positioning assembly 130). Positioning assembly 130 can include shaft 131 comprising two lumens, lumens 132a and 132b shown. Lumen 132a can slidingly receive shaft 111 of infusion assembly 110. Shaft 111 can include lumen 112, a lumen through which material 60 (not shown) can be infused into the patient. Lumen 132b can slidingly receive navigational sensor 197, as shown. Positioning assembly 130 can align navigational sensor 197 axially with shaft 111 of infusion assembly 110, for example as shown in FIG. 4B. FIG. 4B illustrates the distal end of shaft 131 having been positioned (e.g., via a clinician and/or robotic assembly) proximate the stomach wall at a location relative to (e.g., proximate to) the pancreas, with distal end 1118 of shaft 111 having been extended from shaft 132a, through the stomach wall, and into the pancreas. Navigational sensor 197 is shown positioned within lumen 132b of shaft 131.

FIGS. 4C and 4D illustrate a perspective view of the distal portion of positioning assembly 130 and infusion assembly 110, and a sectional view of the distal portion, respectively, where the section is orthogonal to the long axis of shaft 131 of positioning assembly 130. Shaft 111 of infusion assembly 110 can be slidingly received within lumen 132 of shaft 131, as shown. Navigational sensor 197 can be slidingly received within lumen 112 of shaft 111, also as shown. Material 60 (not shown) can be infused into the patient via lumen 112, around navigational sensor 197 (e.g., through the open space within lumen 112 surrounding navigational sensor 197). In these embodiments, navigational sensor 197 can reduce the open space within lumen 112, thus reducing the volume of material 60 that can remain in lumen 112 after an infusion procedure (e.g., reducing waste of material 60).

FIG. 4E illustrates a sectional view of a portion of depositing device 100 and endoscope 80, where the illustrated section is orthogonal to the long axis of shaft 131 of positioning assembly 130 (e.g., a section through distal portion 1317 of positioning assembly 130). Shaft 131 of positioning assembly 130 can be slidingly received within working channel 82 of endoscope 80, as shown. Shaft 111 of infusion assembly 110 can be slidingly received within lumen 132 of shaft 131. Navigational assembly 197 can also be slidingly received within lumen 132, for example alongside shaft 111, also as shown. Material 60 (not shown) can be delivered (e.g., during an infusion process) to the patient via lumen 112 of shaft 111. For example, material 60 can be delivered into the pancreas after shaft 131 has been advanced from working channel 82 of endoscope 80 into the pancreas, as shown in FIG. 4F. FIG. 4F shows the distal end of endoscope 80 positioned proximate the stomach wall relative to the pancreas, with shaft 131 of positioning assembly 130 extending through the stomach wall and into the pancreas, for example when shaft 131 comprises a flexible, needle-like shaft with a sharpened distal end configured to pierce tissue. In some embodiments, shaft 131 is configured to penetrate through the stomach wall but does not extend into the pancreas. At least distal end 1118 of shaft 111 can be extended from lumen 132 into the pancreas (e.g., extended by the operator and/or robotic assembly using deployment control assembly 136 not shown but described herein), such as to deliver material 60 into the pancreas (e.g., deliver material 60 during and/or after the advancement). In some embodiments, navigational sensor 197 can also be extended from lumen 132, for example alongside shaft 111 into the pancreas as shown. In some embodiments, system 10 is configured to monitor the force applied to shaft 111 (e.g., using a sensor, such as functional element 99/199 comprising a force sensor) while shaft 111 is being advanced, for example to detect when the stomach wall has been penetrated. Once penetration of the stomach wall has been detected (e.g., by algorithm 315 via one or more sensor signals), advancement of shaft 111 can be stopped (e.g., robotic force applied by system 10 can be halted), such that shaft 111 does not extend into the pancreas.

Referring now to FIGS. 5A and 5B, sectional views of a “needle in needle” configuration of a delivery device are illustrated, consistent with the present inventive concepts. Depositing device 100 and other components of FIGS. 5A and 5B can be of similar construction and arrangement to similar components described in reference to FIG. 1 and otherwise herein. FIG. 5A shows an inner needle (e.g., an elongate shaft with a needle-like construction, at least in its distal portion), shaft 111 of infusion assembly 110, slidingly received within a lumen of an outer needle (e.g., an elongate shaft with a needle like construction, at least in its distal portion), and lumen 132 of shaft 131 of positioning assembly 130. Material 60 (not shown) can be deposited (e.g., during an infusion procedure described herein) via lumen 112 of shaft 111. In some embodiments, positioning assembly 130 and infusion assembly 110 can be slidingly received within working channel 82 of endoscope 80, as shown. The distal portion of shaft 111 can extend proximally from positioning assembly 130, such that infusion assembly 110 can fluidly attach to a source of material 60, such as to syringe assembly 120, not shown but described herein.

In some embodiments, distal portion 1317 of shaft 131 is configured to be extended through the stomach wall, for example when configured for an operator to extend distal portion 1317 from the distal end of working channel 82 into the space between the stomach and the pancreas, as shown in FIG. 5A. In some embodiments, shaft 131 can extend at least 1 cm, such as at least 2 cm, 3 cm, or 4 cm from the distal end of working channel 82. Additionally or alternatively, shaft 131 can be configured to extend a distance of no more than 8 cm, such as no more than 6 cm, 5 cm, 4 cm, or 3 cm, from the distal end of working channel 82. Distal end 1118 of shaft 111 can be configured to be extended from the distal end of shaft 131 into the pancreas, as shown in FIG. 5B. Lumen 132 of shaft 131 can comprise a cross-sectional profile of sufficient area (e.g., a diameter large enough) to slidingly receive at least a 33 g shaft (e.g., when shaft 111 comprises a 33 g shaft), such as at least a 29 g, a 27 g, a 25 g, or a 23 g shaft. In some embodiments, shaft 111 comprises a needle with a gauge of no more than 23 g, such as no more than 25 g, 27 g, or 29 g.

Referring now to FIGS. 6A and 6B, sectional views of an infusion device with an extendable needle tip are illustrated, consistent with the present inventive concepts. Depositing device 100 and other components of FIGS. 6A and 6B can be of similar construction and arrangement to similar components described in reference to FIG. 1 and otherwise herein. FIG. 6A shows the distal portion of infusion assembly 110. Shaft 111 of infusion assembly 110 can include extendable tip assembly 114, that includes a shaft, shaft 1141 constructed and arranged to extend from distal end 1118 of shaft 111. For example, shaft 1141 can be slidingly located within the distal portion of lumen 112 of shaft 111. Extendable tip assembly 114 can include one or more sealing elements, for example proximal seal 1143a and distal seal 1143b, that surround shaft 1141 and create a fluid tight seal between shaft 1141 and the walls of lumen 112 of shaft 111. Seals 1143 can also maintain the axial alignment of shaft 1141 with lumen 112. Shaft 1141 can comprise one or more lumens fluidly connected to lumen 112, lumen 1142 shown, through which material 60 (not shown) can be infused into the patient (e.g., into the pancreas of the patient).

In some embodiments, shaft 111 of infusion assembly 110 is constructed and arranged to be extended through the stomach wall, for example for an operator to extend distal end 1118 through a delivery device (e.g., through working channel 82 of endoscope 80) into the space between the stomach and the pancreas, as shown in FIG. 6A. Shaft 111 can be extended with extendable tip assembly 114 in a retracted position, as shown. In some embodiments, after shaft 111 has been positioned through the stomach wall and to a location proximate the pancreas, at least distal end 1148 of shaft 1141 can be extended from lumen 112 and into the pancreas. In some embodiments, extendable tip assembly 114 includes one or more mechanical stops, for example stopper 1144 shown, that limits the extension of shaft 1141 from lumen 112. For example, shaft 1141 can be extended until distal seal 1143b abuts stopper 1144, as shown in FIG. 6B. In some embodiments, extendable tip assembly 114 includes an extension control mechanism, linkage 1145 shown. Linkage 1145 can be manipulated (e.g., by an operator and/or robotic assembly of system 10) to control the position of shaft 1141 relative to shaft 111. For example, linkage 1145 can be pushed into lumen 112 to extend shaft 1141, and/or retracted from shaft 111 to retract shaft 1141. Alternatively or additionally, shaft 1141 can be constructed and arranged to extend from lumen 112 via hydraulic or pneumatic pressure, for example by pressure provided by material 60, when material 60 is pushed (e.g., via syringe assembly 120, not shown but described herein) through lumen 112 and into lumen 1142 of shaft 1141. In some embodiments, the pressure caused by material 60 flowing from the larger diameter lumen of shaft 111 into the smaller diameter lumen of shaft 1141 causes shaft 1141 to extend from lumen 112. In some embodiments, linkage 1145 can be used to retract shaft 1141, for example after shaft 1141 has been advanced hydraulically and/or pneumatically. In some embodiments, shaft 1141 can be constructed and arranged to extend from lumen 112 via a mechanical force provided by a biasing assembly, such as a spring activated biasing assembly.

Referring now to FIG. 7, a representative image of the pancreatic anatomy, to be displayed to an operator, is illustrated, consistent with the present inventive concepts. The image displayed can be based on data recorded by system 10 and analyzed by algorithm 315, both described in reference to FIG. 1 and otherwise herein. The image can be displayed to the user using GUI 323 (e.g., GUI 323 displayed to the user on display 322). GUI 323 can display an overlay of the relative location of a positioning assembly (e.g., the distal end of positioning assembly 130 from which shaft 111 will extend). In some embodiments, GUI 323 can display an overlay of a projected path of a deployable shaft (e.g., shaft 111 of infusion assembly 110) into the patient's anatomy prior to the deployment (e.g., prior to manual and/or automatic deployment of shaft 111). The overlay can indicate the depth and position of shaft 111 (e.g., a shaft with a needle-like distal portion) once deployed. Additionally or alternatively, GUI 323 can display various directional and/or positional indicators, such as an indicator of the axis of positioning assembly 130 and/or a guide that is askew from the axis of the positioning assembly (e.g., 45° askew as shown), for example when the needle is configured to exit the positioning assembly at an angle askew from the axis, for example 30° askew as shown. In some embodiments, the distance the shaft 111 can be deployed from positioning assembly 130 can be set by a deployment mechanism, such as deployment control assembly 136 described herein. In some embodiments, system 10 is constructed and arranged to automatically deploy a shaft 111 from positioning assembly 130 (e.g., when automatic deployment is initiated and/or permitted by the operator, such as via input to system 10 via GUI 323). In some embodiments, GUI 323 can display an overlay indicating a “certainty range” indicating to the operator one or more possible errors in the predicted projection path, for example when the shaft 111 is automatically deployed by system 10 in a single advancement (e.g., a single step), where the shaft 111 will extend a full distance (e.g., automatically) along the projected path. In some embodiments, system 10 can comprise one or more sensors (e.g., functional element 99/199) used to determine the distance the shaft 111 will be deployed, for example a proximity sensor configured to determine the location of one or more portions of deployment control assembly 136 from a mechanical stop that limits the deployment distance. In some embodiments, deployment control assembly 136 is constructed and arranged as described in reference to FIGS. 8A-8C and otherwise herein.

In some embodiments, one or more overlays can be displayed on GUI 323 after the shaft 111 has been deployed, such as to indicate the estimated location of the shaft 111, for example when the shaft 111 is not visible to the imaging system, and algorithm 315 is configured to estimate the location of the shaft 111. Alternatively or additionally, shaft 111 can be visualizable by system 10, for example when the shaft 111 (e.g., a shaft with a needle-like distal portion) comprises a coating configured to increase the echogenicity under ultrasonic visualization and/or to increase the radiopacity under fluoroscopic visualization. For example, shaft 111 can comprise a coating, such as a hyperechoic coating. In some embodiments, algorithm 315 is configured to update certainty ranges and/or the location of other prediction overlays based on image data recorded while shaft 111 is deployed.

Referring now to FIGS. 8A-8C, perspective and side sectional views of a depositing device are illustrated, consistent with the present inventive concepts. Depositing device 100 and other devices of system 10 of FIGS. 8A-8C can be of similar construction and arrangement to similar devices described in reference to FIG. 1 and otherwise herein. Depositing device 100 can comprise positioning assembly 130 including deployment control assembly 136 which is constructed and arranged to position infusion assembly 110 relative to shaft 131 of device positioning assembly 130, and also to control the amount of advancement of infusion assembly 110 relative to shaft 131 (e.g., to control the relative position of shaft 111 within lumen 132 of shaft 131). Deployment control assembly 136 comprises a hollow, elongate core, hub 1361 shown, that slidingly receives a first positioner, cap 1362, and a second positioner, cap 1363. Cap 1362 can be slidingly positioned on the top portion of hub 1361 (as shown), and it can slidingly receive shaft 111 of infusion assembly 110, such as a shaft 111 comprising at least a distal portion with a needle-like construction, and a threaded connector 113. Infusion assembly 110 operably engages cap 1362, for example via a threaded engagement of connector 113, such that depositing shaft 111 moves relative to hub 1361 in unison with cap 1362.

Deployment control assembly 136 can include a ring portion, stopper 1364, which can be slidingly received on the top portion of hub 1361, and stopper 1364 can be adjustable (e.g., by an operator), such as to adjust the distal-most allowable position of cap 1362 (and as such depositing element shaft 111 of infusion assembly 110) relative to hub 1361. Stopper 1364 can be temporarily affixed to hub 1361, such as via a fixation element, locking mechanism 1365 shown. In some embodiments, deployment control assembly 136 is constructed and arranged such that cap 1362 is prevented from unintentionally moving proximally (e.g., after distal advancement) along hub 1361. Deployment control assembly 136 can include a motion resisting element, lock 1367. In some embodiments, lock 1367 comprises a set of magnets (e.g., as shown) positioned on the distal end of cap 1362 and proximal end of stopper 1364, such that when cap 1362 is fully advanced (fully distally advanced), the magnets of lock 1367 prevent cap 1362 from retracting proximally without sufficient force being applied (e.g., by the operator) to retract cap 1362 (e.g., to overcome the magnetic force). Alternatively or additionally, lock 1367 can comprise a thumb screw (not shown) and/or other locking element configured to lock cap 1362 in an advanced position. In some embodiments, lock 1367 comprises a thumb screw comprising a wing-nut-like shape, such that the shape of the thumb screw can provide a visual indicator to the operator (e.g., a visual indicator of the current condition of lock 1367). In other embodiments, lock 1367 can comprise a ratcheting mechanism (not shown), such as a ratcheting mechanism configured to allow incremental advancement of cap 1362, but to prevent retraction (e.g., without releasing the ratchet mechanism). In some embodiments, lock 1367 comprises a spring-loaded locking mechanism (not shown), such as a locking mechanism that prevents (or at least limits) motion of cap 1362 relative to hub 1361 in either and/or both directions unless the locking mechanism is actively released (e.g., an operator overcomes the spring force to temporarily release the lock). In some embodiments, lock 1367 provides sufficient locking force to maintain the position of cap 1362 when deployment control assembly 136 is held in an “upside-down” position.

Cap 1363 can be slidingly positioned on the bottom portion of hub 1361 (as shown), and it can slidingly receive shaft 131, which is attached to the distal end of hub 1361. The proximal end of endoscope 80 can operably engage cap 1363, for example via a threaded engagement, such that shaft 131 moves relative to endoscope 80 (e.g., within working channel 82 of endoscope 80) as cap 1363 moves relative to hub 1361. Cap 1363 can be temporarily affixed to hub 1361, such as via a fixation element, locking mechanism 1366 shown.

In some embodiments, depositing device 100 includes stylet 115 shown, which can be slidingly received within shaft 111 of infusion assembly 110, for example prior to insertion of shaft 111 into tissue. After insertion into tissue, stylet 115 can be removed to allow a material (e.g., material 60) to be infused into a target region of the patient, via one or more deposit sites, via infusion assembly 110. Stylet 115 can prevent or at least limit undesired material (e.g., tissue) from entering lumen 112 of shaft 111, such as while shaft 111 is advanced through the patient (e.g., through patient tissue). Alternatively or additionally, infusion assembly 110 can be primed (e.g., filled with material such as material 60) prior to insertion of shaft 111 into the patient.

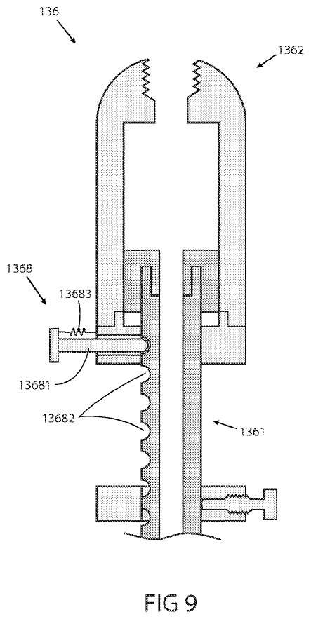

Referring now to FIG. 9, a sectional view of a locking mechanism of a deployment assembly is illustrated, consistent with the present inventive concepts. Deployment control assembly 136 and other components of FIG. 9 can be of similar construction and arrangement to similar components described in reference to FIG. 1 and otherwise herein. Deployment control assembly 136 includes cap 1362 configured to slidingly receive shaft 111 of infusion assembly 110 (not shown) and fixedly attach to proximal portion 1112 of shaft 111, such that shaft 111 can be advanced and/or retracted relative to hub 1361 as cap 1362 is advanced and/or retracted. Hub 1361 includes a lumen that slidingly receives shaft 111. Deployment control assembly 136 can fixedly attach to a positioning assembly, such as positioning assembly 130 described herein (not shown), such that the movement of cap 1362 can control the deployment of shaft 111 from positioning assembly 130.

In some embodiments, deployment control assembly 136 includes a locking assembly 1368 configured to releasably lock the position of cap 1362 relative to hub 1361 (e.g., to prevent accidental deployment of shaft 111 from positioning assembly 130). Locking assembly 1368 can include a pin, pin 13681 shown, that is slidingly received within a lumen that is perpendicular to the translation axis of cap 1362. Hub 1361 can include a series of locking points, recesses 13682, into which pin 13681 can extend to lock the position of cap 1362 relative to hub 1361. In some embodiments, locking assembly 1368 can include a biasing assembly, spring 13683, that biases pin 13681 in a locked position (e.g., where pin 13681 is engaged with a recess 13682). In some embodiments, an operator of system 10 can retract pin 13681 from recess 13682 to release cap 1362 to translate relative to hub 1361 (e.g., to deploy shaft 111 from positioning assembly 130).

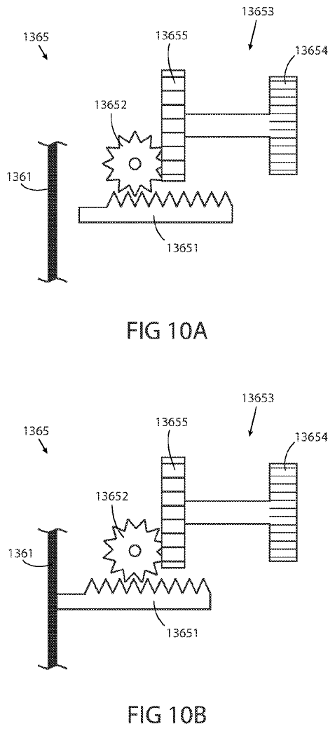

Referring now to FIGS. 10A-10B, sectional views of a rack and pinion locking mechanism are illustrated, consistent with the present inventive concepts. As described in reference to FIG. 8C and otherwise herein, deployment control assembly 136 can include one or more locking mechanisms, such as locking mechanisms 1365 and/or 1366 shown that temporarily fix the position of stopper 1364 and cap 1363 to hub 1361 as described herein. In some embodiments, one or more of these locking mechanisms comprise a rack and pinion-based locking mechanism, such as is shown in FIGS. 10A and 10B. Locking mechanism 1365 can include a locking member, rack pin 13651, that frictionally engages hub 1361 when in a locked position, as shown in FIG. 10B. Rack pin 13651 can include a linear gear that operably engages with a pinion gear, pinion 13652 shown. In some embodiments, pinion 13652 operably engages with an operator control portion, thumb screw assembly 13653. Thumb screw assembly 13653 can include a thumb screw 13654 that is fixedly attached to gear 13655. Gear 13655 can operably engage pinion 13652, such that rotation of thumb screw 13654 (e.g., rotation by an operator of system 10) rotates pinion 13652, and in turn translates rack pin 13651 (e.g., toward and/or away from hub 1361). In some embodiments, the various gears of locking mechanism 1365 can be designed such that locking mechanism 1365 transitions from a fully unlocked state to a fully locked state with no more than one full rotation (i.e. no more than 360° rotation) of thumb screw 13654, such as when this transition requires no more than one half rotation (i.e. no more than 180° rotation), or one quarter rotation (i.e. no more than 90° rotation). In some embodiments, thumb screw 13654 comprises an asymmetric shape, and/or one or more markings that indicate the locked and unlocked positions of thumb screw 13654.

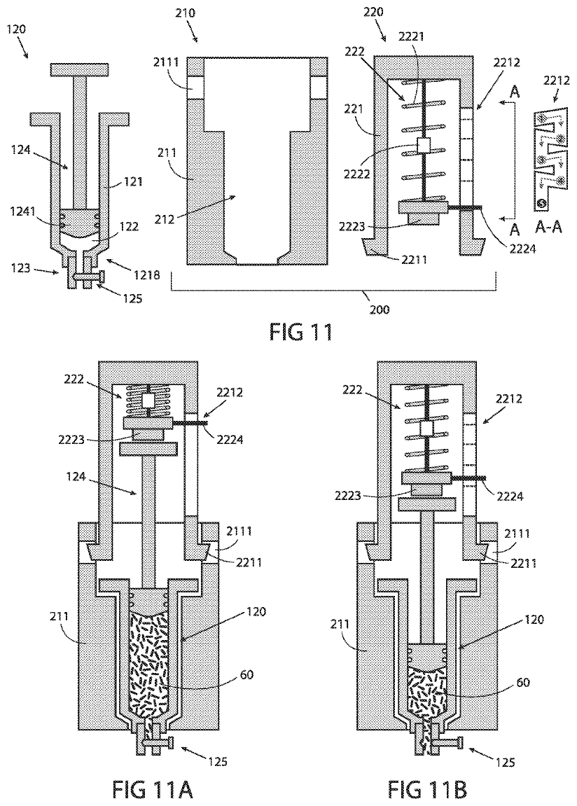

Referring now to FIG. 11, sectional views of various components of a syringe and a syringe drive assembly including a spring and a damper are illustrated, consistent with the present inventive concepts. FIG. 11 also shows a side view from reference A-A shown, of a portion of a locking mechanism. Referring additionally to FIGS. 11A and 11B, sectional views of the syringe drive assembly operably attached to the syringe are illustrated, consistent with the present inventive concepts. Drive assembly 200 and other components of FIG. 11 can be of similar constructions and arrangement to similar components described in reference to FIG. 1 and otherwise herein.

Syringe assembly 120 can include barrel 121 that slidingly receives seal 1241 of plunger 124. Chamber 122 is located within barrel 121 between seal 1241 and connector 123 located at distal end 1218 of barrel 121. In some embodiments, syringe assembly 120 comprises one or more valves, such as valve 125 shown operably connected to connector 123. Valve 125 can control the flow of material 60 from chamber 122, for example when syringe assembly 120 is attached to a spring actuated drive assembly 200, prior to the intended delivery of material 60.

Drive assembly 200 can include syringe adapter 210 that removably attaches to syringe assembly 120. Syringe adapter 210 can include housing 211 that surrounds a space that receives barrel 121 of syringe assembly 120, such as chamber 212 shown. Chamber 212 can be constructed and arranged such that barrel 121 can be fixedly positioned therein, such that motive assembly 220 can actuate plunger 124 relative to barrel 121 and syringe adapter 210. In some embodiments, motive assembly 220 can removably attach to housing 211. Housing 211 can include one or more fixation elements, such as recesses 2111 shown that are configured to removably attach to a portion of motive assembly 220 as described herein.

Motive assembly 220 can include a housing, housing 221, that surrounds a biasing assembly, biasing assembly 222. Housing 221 can removably attach to syringe adapter 210, for example to position biasing assembly 222 relative to syringe assembly 120 (e.g., when syringe assembly 120 is positioned within chamber 212 of syringe adapter 210, such as is shown in FIGS. 11A and 11B). Housing 221 can include one or more locking mechanisms, such as projections 2211 shown. Projections 2211 can be lockingly engaged with recesses 2111 of housing 211, for example when housing 221 plastically deforms to allow projections 2211 to enter chamber 212, and elastically reforms when projections 2211 are aligned with recesses 2111, locking the relative positions of housings 211 and 221.

Biasing assembly 222 can include one or more force applying elements, spring 2221 shown. Biasing assembly 222 can include a translating element, pusher 2223 shown, that applies force (e.g., the force provided by spring 2221) to plunger 124 of syringe assembly 120. In some embodiments, biasing assembly 222 incudes a force dampening mechanism, damper 2222 shown, that modifies the force applied by spring 2221 to plunger 124. In some embodiments, biasing assembly 222 includes a control mechanism, control arm 2224, that allows an operator to adjust or otherwise control the force applied by biasing assembly 222 to plunger 124. Control arm 2224 can extend through an opening of housing 221, position controller 2212, that allows the operator to lockingly adjust the position of pusher 2223 relative to housing 221. View A-A shows a possible shape of position controller 2212, illustrating how the operator can position control arm 2224 in various locked locations. For example, position controller 2212 can comprise a shape configured to prevent control arm 2224 (and pusher 2223) from advancing distally (as driven by biasing assembly 222) without the operator transitioning control arm 2224 from a locked position (e.g., positions 1-4 shown) to a position where control arm 2224 can translate distally as shown.

FIG. 11A shows syringe assembly 120 with material 60 positioned within chamber 122. Syringe assembly 120 has been positioned within chamber 212 of housing 211, and housing 221 of motive assembly 220 has been attached to housing 211 of syringe adapter 210. Spring 2221 of biasing assembly 222 has been compressed by plunger 124. In FIG. 11A, valve 125 is in a closed position, preventing material 60 from exiting syringe assembly 120. In some embodiments, spring 2221 is compressed (e.g., as shown in FIG. 11A) prior to attachment of motive assembly 220 to syringe adapter 210, such as by positioning control arm 2224 in position 1 (e.g., position 1 shown in view A-A of FIG. 11) of position controller 2212. FIG. 11B shows plunger 124 partially depressed by biasing assembly 222, where valve 125 is in an open position, allowing material 60 to flow from syringe assembly 120 (e.g., through infusion assembly 110, not shown, but described herein and configured to be operably attached to connector 123 and/or valve 125).

In some embodiments, motive assembly 220 provides an actuation force to plunger 124 of at least 0.5 lbf, such as at least 21 bf, and/or no more than 51 bf, such as no more than 4 lbf, for example approximately 1.75 lbf. In some embodiments, damper 2222 is constructed and arranged to provide a constant and/or near constant force from biasing assembly 222 over the length of travel of pusher 2223. For example, spring 2221 can comprise a spring force greater than the desired biasing force (e.g., spring 2221 can comprise a spring force of at least 10 lbf), such that when spring 2221 is fully or near-fully extended (e.g., at the end of the stroke of plunger 124), the spring force is sufficient to provide the biasing force necessary. Damper 2222 can be configured to resist the spring force of spring 2221 to provide a constant force across the entire stroke of plunger 124. In some embodiments, biasing assembly 222 can be “reset” (e.g., spring 2221 can be compressed and control arm 2224 positioned in locking position 1) manually (e.g., by an operator of system 10) or using a motorized and/or mechanical advantage-based assembly, such as a motorized assembly of console 300 and/or a functional element 99 of system 10. In some embodiments, damper 2222 comprises a geared mechanism that is constructed and arranged to maintain a constant biasing force as the spring force provided by spring 2221 varies (e.g., as spring 2221 decompresses). In some embodiments, spring 2221 comprises two, three, or more springs, such as springs of varying types (e.g., compression and/or extension springs), various spring forces, and/or various orientations (e.g., relative to pusher 2223). In some embodiments, spring 2221 can comprise a constant force spring, such as a spring that provides a constant (or near constant) force over at least a portion of the length of travel of pusher 2223.

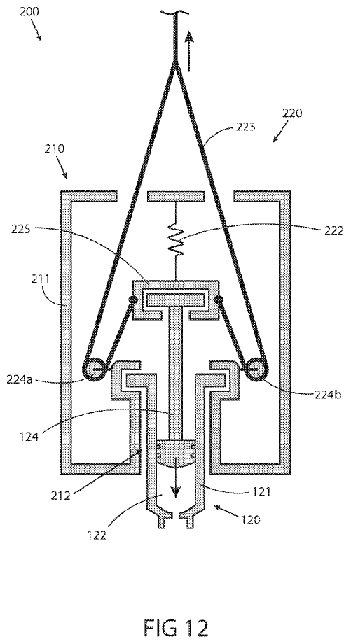

Referring now to FIG. 12, a sectional view of a syringe drive assembly including a pull wire mechanism is illustrated, consistent with the present inventive concepts. Drive assembly 200 and other components of FIG. 12 can be of similar constructions and arrangement to similar components described in reference to FIG. 1 and otherwise herein. Drive assembly 200 can include syringe adapter 210 including housing 211. Chamber 212 of housing 211 can removably receive and fix the position of syringe assembly 120 to housing 211. Motive assembly 220 can include one or more pull wires or other mechanical linkages, linkage 223 shown, that are attached to connector 225. Connector 225 attaches to plunger 124 of syringe assembly 120, such that translation of connector 225 causes the translation of plunger 124 (e.g., to depress plunger 124 to push material 60 from chamber 122).

Motive assembly 220 can include one or more force direction-modifying elements, pulleys 224a,b shown. Linkage 223 can include two ends, as shown, each looped around pulleys 224a,b, and fixedly attached to connector 225, such that force applied to linkage 223 away from syringe assembly 120 (upwards as shown) causes plunger 124 to translate into chamber 122 (e.g., to translate opposite the direction of force applied to linkage 223). In some embodiments, linkage 223 can be configured such that force applied in the opposite direction (downwards as shown) causes plunger 124 to translate into chamber 122 (e.g., to translate in the direction of the force applied). In some embodiments, biasing assembly 222 comprises one or more springs (one shown) that bias connector 225 proximally, out of chamber 122. In some embodiments, motive assembly 220 comprises a motor and/or a linear drive assembly that applies a force to linkage 223 to depress plunger 124. Alternatively or additionally, an operator of system 10 can manually apply a pulling force to linkage 223 to depress plunger 124. In some embodiments, when the force is removed from linkage 223, biasing assembly 222 can apply a force to retract plunger 124. In some embodiments, connector 225 and/or chamber 212 are constructed and arranged to evenly distribute force loads to plunger 124 and/or barrel 121, preventing or at least limiting torque from being applied which may cause plunger 124 to bind within chamber 212.

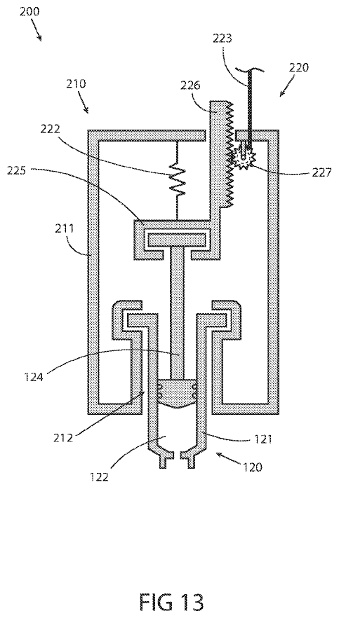

Referring now to FIG. 13, a sectional view of a syringe drive assembly including a rack and pinion mechanism is illustrated, consistent with the present inventive concepts. Drive assembly 200 and other components of FIG. 13 can be of similar constructions and arrangement to similar components described in reference to FIG. 1 and otherwise herein. Drive assembly 200 can include syringe adapter 210 including housing 211. Chamber 212 of housing 211 can removably receive and fix the position of syringe assembly 120 to housing 211. Motive assembly 220 can include a rack and pinion mechanism, for example rack gear 226 fixedly attached to connector 225. Motive assembly 220 can include pinion 227, located proximate rack gear 226 such that rotation of pinion 227 causes translation of rack gear 226, which in turn translates plunger 124. In some embodiments, linkage 223 is wound about an axle of pinion 227, such that a pulling force applied to linkage 223 causes the rotation of pinion 227, and the translation of plunger 124. Alternatively or additionally, pinion 227 can be operably attached to a motor that controls the rotation of pinion 227. In some embodiments, biasing assembly 222 comprises a spring configured to bias plunger 124 in a retracted position (e.g., to return plunger 124 to a retracted position when force is removed from pinion 227, and/or to reduce the force required to retract plunger 124).

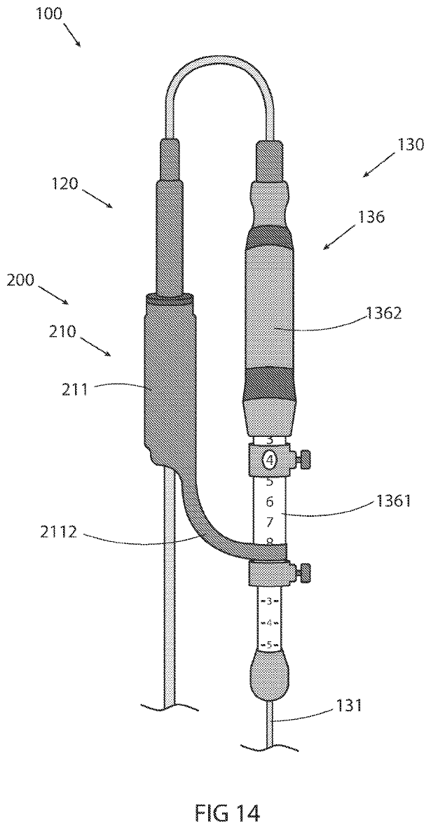

Referring now to FIG. 14, side views of the handle portion of a depositing device are illustrated, consistent with the present inventive concepts. Depositing device 100 and other components of FIG. 14 can be of similar construction and arrangement to similar components described in reference to FIG. 1 and otherwise herein. Drive assembly 200 can include syringe adapter 210 that operably attaches to syringe assembly 120, for example as described in reference to FIG. 11 and otherwise herein. In some embodiments, housing 211 of syringe adapter 210 can include an assembly for connecting to another component of system 10, connector 2112 shown. Connector 2112 can removably attach housing 211 to a portion of positioning assembly 130, for example deployment control assembly 136. Deployment control assembly 136 can include various components that can be translated relative to each other and/or other components of system 10. For example, deployment control assembly 136 can include hub 1361 and cap 1362, each shown and described herein. Cap 1362 can translate (vertically as shown) along hub 1361, such as to control the deployment of infusion assembly 110 (not shown in FIG. 14). Hub 1361 can be positioned (e.g., by an operator) in a relatively stationary position, such as proximate handle 81 of endoscope 80 (not shown, but described herein), for example when shaft 131 of positioning assembly 130 is introduced into the patient via working channel 82 of endoscope 80. Connector 2112 can be attached to a portion of deployment control assembly 136 other than cap 1362, such as to hub 1361, as shown, such that cap 1362 can freely translate along hub 1361 without the mass of syringe adapter 210 (and syringe assembly 120) affecting the position of cap 1362 (e.g., the mass of syringe adapter 210 does not cause unwanted downward translation of cap 1362 along hub 1361).

Connector 2112 can be removably attached to a stationary portion of positioning assembly 130, for example to hub 1361 as shown. In some embodiments, connector 2112 is rotatably attached to hub 1361, such that drive assembly 200 can be rotated about deployment control assembly 136, for example such that the operator of system 10 can position drive assembly 200 in an “out of the way” location (e.g., in an operator preferred orientation). Drive assembly 200 and connector 2112 can be constructed and arranged such that manipulation of cap 1362 is unimpeded by drive assembly 200 through a stroke length of at least 7.5 cm.

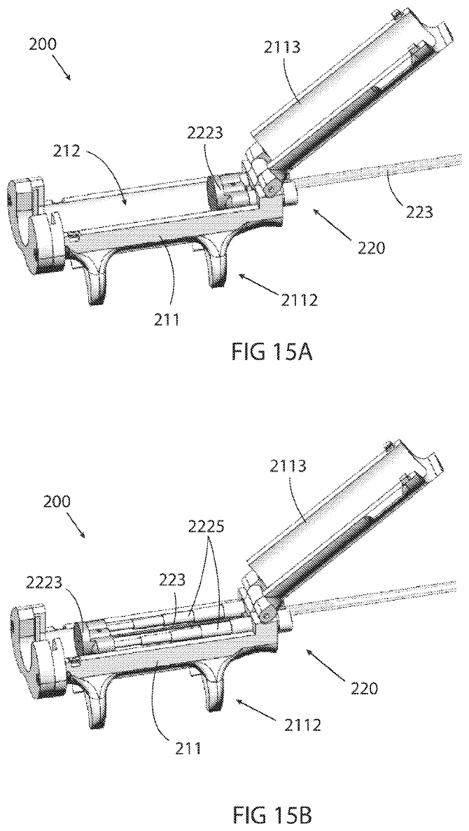

Referring now to FIGS. 15A and 15B, perspective views of a syringe drive assembly are illustrated, consistent with the present inventive concepts. Drive assembly 200 and other components of FIGS. 15A and 15B can be of similar construction and arrangement to similar components described in reference to FIG. 1 and otherwise herein. Drive assembly 200 can include housing 211 with chamber 212 that receives syringe assembly 120, not shown but described herein. In some embodiments, housing 211 can include a removable cover, cover 2113 shown. In some embodiments, cover 2113 is hinged to housing 211 as shown.

Motive assembly 220 can include pusher 2223 positioned within housing 211. Pusher 2223 can drive plunger 124 of syringe assembly 120 to depress plunger 124. Motive assembly 220 can include linkage 223 that applies a force to pusher 2223 (e.g., when linkage 223 comprises a push rod that applies a pushing force to pusher 2223). In some embodiments, linkage 223 is manually translated by an operator of system 10. Alternatively or additionally, linkage 223 can operably attach to a linear translator or other force applying element of motive assembly 220 (e.g., a linear translator located in console 300 and operably attached to drive assembly 200 with one or more linkages, such as linkage 223). In some embodiments, linkage 223 comprises a nickel titanium wire extending through a tube, such as a PTFE tube. The wire and tube arrangement of linkage 223 can provide an approximately 1:1 translation of the proximal and distal ends of the wire relative to the tube of linkage 223. In some embodiments, motive assembly 220 includes one or more guide elements, guides 2225 shown. Guides 2225 can prevent or at least limit buckling of linkage 223 as pusher 2223 is translated distally through housing 211 (as shown in FIG. 15B). In some embodiments, guides 2225 comprise a telescoping geometry, as shown (e.g., such that guides 2225 do not extend from housing 211 when pusher 2223 is in a proximal position, as shown in FIG. 15A). In some embodiments, guides 2225 comprise telescoping coiled sheets, such as coiled sheets of spring steel (e.g., spring steel at least 0.0005″ thick and no more than 0.003″ thick).

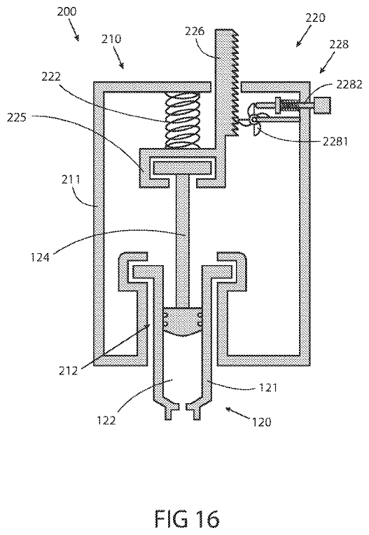

Referring now to FIG. 16, a sectional view of a syringe drive assembly is illustrated, consistent with the present inventive concepts. Drive assembly 200 and other components of FIG. 16 can be of similar constructions and arrangement to similar components described in reference to FIG. 1 and otherwise herein. Drive assembly 200 can include syringe adapter 210 including housing 211. Chamber 212 of housing 211 can removably receive and fix the position of syringe assembly 120 to housing 211. Motive assembly 220 can include biasing assembly 222 comprising a spring that applies a biasing force to connector 225 to depress plunger 124. Motive assembly 220 can include rack gear 226 extending from connector 225 as shown. Motive assembly 220 can include locking assembly 228 that is constructed and arranged to resist biasing assembly 222 and control the depression of plunger 124. Locking assembly 228 can include gear 2281 that engages rack gear 226. Locking assembly 228 can include a spring-loaded stopping mechanism, stopper 2282, that can be retracted by an operator to release gear 2281 and allow rack gear 226 to advance (e.g., to allow biasing assembly 222 to depress plunger 124).

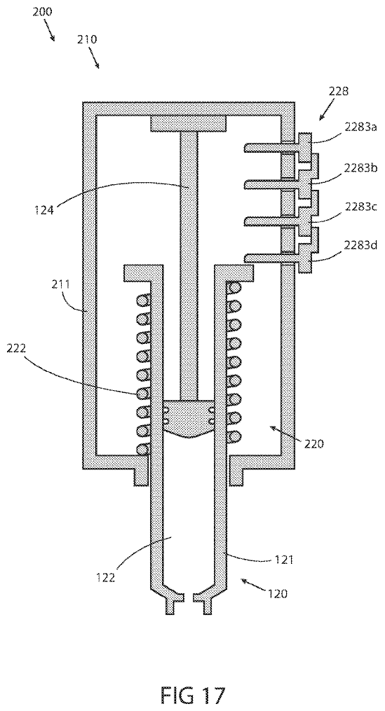

Referring now to FIG. 17, a sectional view of a syringe drive assembly is illustrated, consistent with the present inventive concepts. Drive assembly 200 and other components of FIG. 17 can be of similar constructions and arrangement to similar components described in reference to FIG. 1 and otherwise herein. Drive assembly 200 can include syringe adapter 210 including housing 211, as shown. Housing 211 can receive syringe assembly 120, for example with biasing assembly 222 comprising a spring positioned about barrel 121 and providing a force that biases barrel 121 toward the proximal wall of housing 211. In some embodiments, the proximal wall of housing 211 can removably attach to the proximal end of plunger 124. Alternatively or additionally, the proximal wall of housing 211 can provide an opposing force to plunger 124, such that biasing assembly 222 of motive assembly 220 drives barrel 121 onto plunger 124 to drive material 60 from chamber 122. In some embodiments, locking assembly 228 comprises one or more removable pins, pins 2283a-d shown. Pins 2283 can be removed from housing 211 to allow barrel 121 to translate proximally onto plunger 124. In some embodiments, pins 2283 are constructed and arranged such that they can only be removed in a pre-determined order (e.g., a stepwise, sequential order), such as to prevent an unintended amount of material 60 from being delivered to the patient. For example, pins 2283 can include flanges configured to prevent proximal pins from being removed out of order.

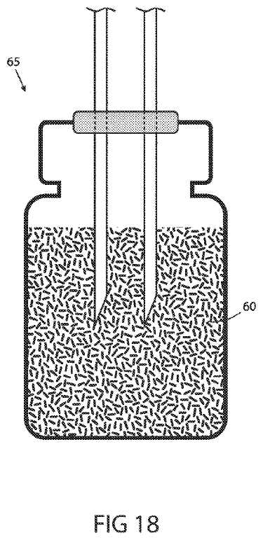

Referring now to FIG. 18, a sectional view of a material storage and delivery assembly is illustrated, consistent with the present inventive concepts. Deposit device 100 and other components of FIG. 18 can be of similar constructions and arrangement to similar components described in reference to FIG. 1 and otherwise herein. In some embodiments, system 10 includes a storage container, vial 65 shown, for the transportation and storage of material 60. In some embodiments, material 60 is infused via infusion assembly 110 without transferring material 60 to syringe assembly 120. For example, vial 65 can comprise a container that is and/or can be pressurized, such as a container that is constructed and arranged to receive a proximal end of shaft 111 (or a second shaft or tube fluidly connected to the proximal end 1112 of shaft 111) and a pressurization source. Pressure within vial 65 can force material 60 from vial 65 and into lumen 112 of shaft 111. In some embodiments, vial 65 comprises a septum through which one or more shafts (e.g., shafts with needle-like distal portions) can be inserted, as shown.

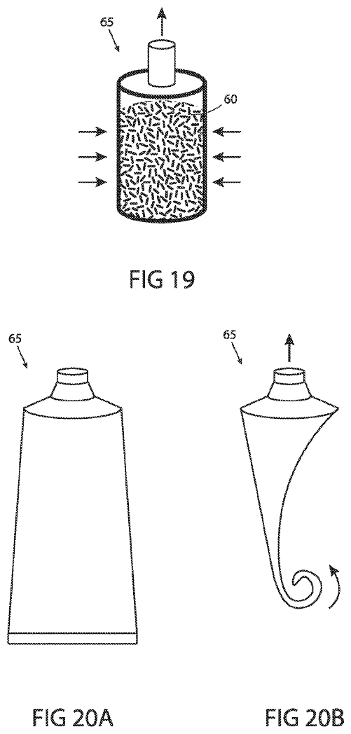

Referring additionally to FIG. 19, a sectional view of a material storage and delivery assembly is illustrated, consistent with the present inventive concepts. In some embodiments vial 65 can include a “squeeze bottle” type design. Compression of vial 65 (e.g., manual compression or mechanical compression by a functional element 99 of system 10 configured to compress vial 65) can force material 60 from vial 65 into lumen 112 of shaft 111 for infusion into the patient. In some embodiments, vial 65 includes a one-way valve to prevent material 60 from flowing backwards into vial 65. In some embodiments, the force applied to compress vial 65 can be adjusted to adjust the flowrate of material 60 from vial 65.

Referring additionally to FIGS. 20A and 20B, side views of a material storage and delivery assembly are illustrated, consistent with the present inventive concepts. In some embodiments, vial 65 can include a rolling bag (e.g., toothpaste container) design. As shown in FIG. 20B, rolling the end of vial 65 can force material 60 from vial 65 into lumen 112 of shaft 111 for infusion into the patient. In some embodiments, functional element 99 of system 10 comprises an assembly configured to mechanically “roll” vial 65. The rolling speed can be adjusted to adjust the flowrate of material 60 from vial 65.

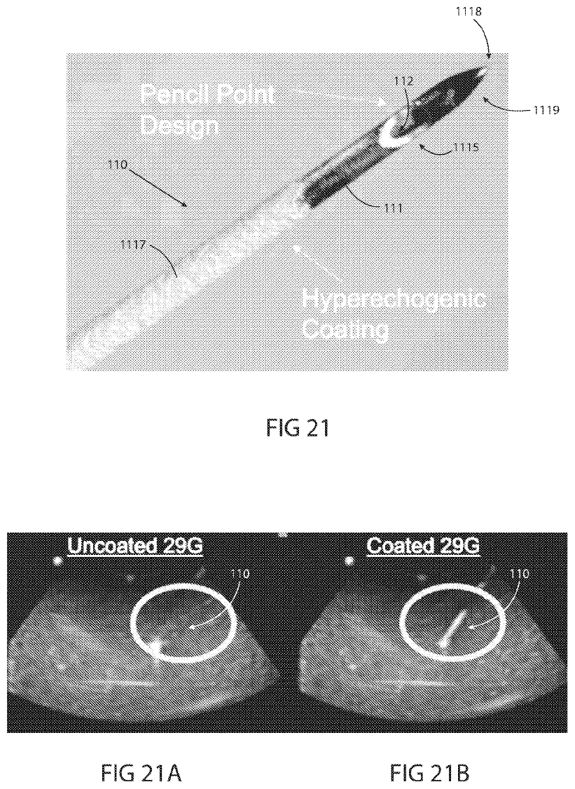

Referring now to FIGS. 21, 21A, and 21B, a photograph of the distal portion of a shaft including a hyperechogenic coating, and examples of coated and uncoated needles as viewed using ultrasonic imaging are illustrated, respectively, consistent with the present inventive concepts. Infusion assembly 110 and other components of FIGS. 21, 21A, and 21B can be of similar construction and arrangement to similar components described in reference to FIG. 1 and otherwise herein. As described herein, infusion assembly 110 can comprise shaft 111, such as a needle-like shaft, with lumen 112 passing therethrough. In some embodiments, a tip-portion of shaft 111, tip 1119, can comprise a “pencil-point” geometry, as shown, such as when shaft 111 comprises a conical distal end. Alternatively or additionally, tip 1119 can comprise a beveled geometry, such as a geometry selected from the group consisting of: a long bevel; a medium bevel; a short bevel; a multi-bevel; a scalpel bevel; and combinations of these. The distal portion of shaft 111 can comprise one or more holes or other openings (e.g., an array of holes), such as port 1115 shown. Port 1115 can comprise one, two, three or more openings through a side wall of shaft 111, such that lumen 112 exits the side wall of shaft 111 at a location proximal to distal end 1118 (e.g., and a location proximal to tip 1119), also as shown. Port 1115 can exit the side of shaft 111, as shown, and/or port 1115 can exit distal end 1118 of shaft 111. In some embodiments, port 1115 can comprise two, three, or more side facing openings (e.g., openings through the side wall of shaft 111), such as multiple openings positioned circumferentially about shaft 111 and/or multiple openings positioned along a length of the distal portion of shaft 111. In some embodiments, shaft 111 can comprise one or more porous segments, such as a porous segment of shaft 111 positioned near the distal portion of shaft 111. The porous segment can be configured as one or more ports 1115 that are constructed and arranged such that material 60 can exit lumen 112 via the porosity of the segment. Port 1115 can comprise an opening with a cross sectional geometry selected from the group consisting of: round; oval; rectangular; trapezoidal; and combinations of these. In some embodiments, each port 1115 comprises a cross-sectional area of no less than the cross-sectional area of lumen 112, such as at least 25% larger than the cross-sectional area of lumen 112. Alternatively or additionally, the total cross-sectional area of all of ports 1115 (e.g., when shaft 111 comprises two or more ports 1115) is no less than the cross-sectional area of lumen 112, such as at least 25% larger than the cross-sectional area of lumen 112. In some embodiments, port 1115 comprises a major diameter of at least 0.4 cm, such as at least 0.6 cm, 0.8 cm, or 1.0 cm. In some embodiments, the distal end of port 1115 (e.g., the distal end of the most distal port 1115 when port 1115 comprises multiple ports) is less than 1 cm from distal end 1118 of shaft 111, such as less than 0.8 cm, 0.6 cm, of 0.4 cm.

One or more portions of shaft 111 can each comprise one, two, or more coatings, other surface treatments, and/or other surface modifications, coating 1117 shown. Coating 1117 can comprise a coating configured to increase the visibility of shaft 111 when imaged using one or more imaging modalities. For example, coating 1117 can comprise a hyperechogenic coating configured to increase the echogenicity of shaft 111 when imaged using ultrasonic imaging. Alternatively or additionally, coating 1117 can comprise a radiopaque coating configured to increase the visibility using X-ray imaging. FIGS. 21A and 21B show examples of uncoated and coated, respectively, 29 G needles imaged using ultrasonic imaging. As can be seen in the figures, coating 1117 increases the visibility of shaft 111 in FIG. 21B compared to the otherwise similar shaft 111 shown in FIG. 21A. In some embodiments, coating 1117 comprises an hyperechogenic coating such as Sono-Coat™ provided by Encapson B. V. In some embodiments, coating 1117 can comprise a biocompatible coating and/or a temperature-resistant coating, such as a coating configured to withstand temperatures of at least 100 °C without adversely affecting the coating. In some embodiments, coating 1117 can include two or more coatings, such as when coating 1117 comprises both a hyperechogenic coating and a second coating overcoated on the hyperechogenic coating, such as when the second coating comprises a lubricious, anti-microbial, and/or anti-thrombogenic coating. In some embodiments, the overcoating of coating 1117 does not decrease the echogenicity provided by the hyperechogenic coating 1117. In some embodiments, coating 1117 comprises a thickness of no more than 0.015 mm, such as less than 0.03 mm, 0.06 mm, or 0.08 mm.

In some embodiments, shaft 111 comprises a shaft with a diameter no larger than 25 G, such as a diameter of no more than 26 G, 27 G, 28 G, 29 G, or 30 G. One or more surface and/or other portions of shaft 111 can comprise coating 1117. In some embodiments, coating 1117 is applied to the outer surface of shaft 111 proximate but proximal to port 1115 as shown. The distal end of coating 1117 can terminate at a location that is no more than 1.00 cm from distal end 1118 of shaft 111, such as no more than 0.75 cm, 0.50 cm, or 0.25 cm. In some embodiments, coating 1117 comprises a length of at least 0.25 cm, such as at least 0.50 cm, 0.75 cm, or 1.00 cm. Additionally or alternatively, coating 1117 can comprise a length of no more than 1.50 cm, such as no more than 1.25 cm, 1.00 cm, or 0.75 cm. As described herein, shaft 111 can comprise one, two, or more materials, such as stainless steel, a cobalt-chromium alloy, a nickel-titanium alloy, and/or a non-metallic material, such as PEEK.

In some embodiments, infusion assembly 110 can include a single-part shaft (e.g., shaft 111 comprises a single-part shaft), such as when shaft 111 comprises a single-part shaft that extends the length of depositing device 100 (not shown, but described herein), such as when shaft 111 is configured to be inserted into shaft 131 of positioning assembly 130, to pass through proximal portion 1312, and to exit shaft 131 proximate tissue to be infused with material 60. Alternatively or additionally, infusion assembly 110 can comprise a multi-part shaft (e.g., shaft 111 comprises a multi-part shaft). For example, a distal shaft of shaft 111 can comprise a needle-like shaft as described herein, and a proximal shaft of shaft 111 can comprise a tube that extends proximally from the distal shaft. The distal shaft can comprise a length of at least 5 cm, such as at least 6 cm, 7 cm, or 8 cm. A proximal shaft of shaft 111 can comprise a similar and/or a dissimilar material from a distal shaft of shaft 111. For example, a proximal shaft of shaft 111 can comprise a material that minimizes adsorption of material 60, such as when shaft 111 comprises a material that minimizes viral adsorption (e.g., when material 60 comprises a viral vector to be delivered to the tissue). For example, shaft 111 (e.g., a proximal shaft of shaft 111) can comprise a material including cyclic olefin polymers (COP) and/or other materials configured to minimize adsorption. In some embodiments, a proximal shaft of shaft 111 comprises a larger diameter (e.g., a larger inner diameter and/or a larger outer diameter) than the diameter of a distal shaft of shaft 111, for example the proximal shaft can comprise a shaft at least 1 G larger than the distal shaft, such as at least 2 G, or 4 G larger.

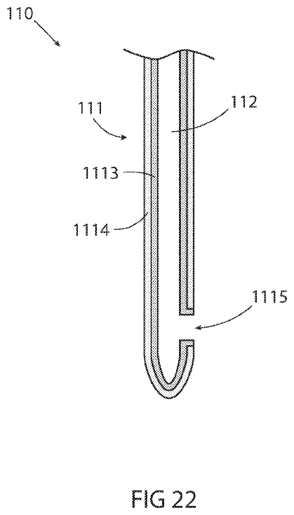

Referring now to FIG. 22, a sectional view of an embodiment of an infusion assembly is illustrated, consistent with the present inventive concepts. Infusion assembly 110 and other components of FIG. 22 can be of similar construction and arrangement to similar components described in reference to FIG. 1 and otherwise herein. Infusion assembly 110 can be configured to prevent and/or at least limit viral loss, such as viral adsorption, as treatment material 60 (not shown but described herein) is delivered to the patient via infusion assembly 110, as described herein. Infusion assembly 110 can comprise shaft 111, such as a needle-like shaft, with lumen 112 passing therethrough. In some embodiments, shaft 111 comprises a multi-layer shaft, such as a shaft including at least inner layer 1113, and two or more outer layers, outer layer 1114 shown.

In some embodiments, the distal portion of shaft 111 comprises one or more holes or other openings (e.g., an array of holes), such as port 1115 shown and described herein. In some embodiments, shaft 111 comprises a blunted tip, such as a “bullet” or a “pencil point” tip, as shown and described herein. Port 1115 can comprise a hole through a side wall of shaft 111.

Inner layer 1113 can comprise at least the inner surface of the walls of lumen 112. In some embodiments, inner layer 1113 is constructed and arranged to limit viral loss, such as when inner layer 1113 comprises a material that is selected to prevent viral loss (e.g., to limit adsorption of treatment material 60 to the walls of inner layer 1113). For example, inner layer 1113 can comprise a material selected from the group consisting of: borosilicate glass; high density polyethylene (HDPE); low density polyethylene (LDPE); crystal zenith; flint glass; polycarbonate (PC); silicone; polypropylene (PP); a PP copolymer; polyethylene terephthalate glycol (PETG); polyolefin; polystyrene; a nylon material; other engineered plastics; acrylic; ABS; PLA; polyvinylidene fluoride; soda-lime glass; lead glass; aluminosilicate glass; silica glass, such as 96% silica glass; fused silica glass; ceramic glass; Saint-Gobain's Tygon® thermoplastic; PEEK; and combinations of these.

In some embodiments, inner layer 1113 comprises a coating of a material, such as a coating that is applied to the luminal surface of lumen 112 extending through outer layer 1114. Inner layer 1113 comprising a coating can be applied to the inner walls of outer layer 1114 of shaft 111during a manufacturing process and/or in a procedural step where infusion assembly 110 is prepared for delivery of treatment material 60 to the patient. In some embodiments, inner layer 1113 comprising a coating can comprise an anti-adhesion material, such as polyionic hydrophilic complex.

In some embodiments, system 10 includes two or more variations of infusion assembly 110, for example different variations where inner layer 1113 comprises a material that is selected to minimize viral loss based on the viral composition of treatment material 60 (e.g., a first variation of infusion assembly 110 for delivery of a first virus, and a second variation of infusion assembly 110 for delivery of a second virus). In some embodiments, treatment material 60 comprises one or more additives, additive 61 described herein. Additive 61 can be configured to limit viral loss of treatment material 60, such as viral loss due to viral adsorption. For example, additive 61 can comprise a buffer solution, such as a buffer solution selected from the group consisting of: poloxamer 188 (P188); polysorbate 20 (PS20); polysorbate 80 (PS80), and combinations of these. In some embodiments, additive 61 can comprise a biocompatible block copolymer with surface-active properties that is added to treatment material 60 as an excipient.

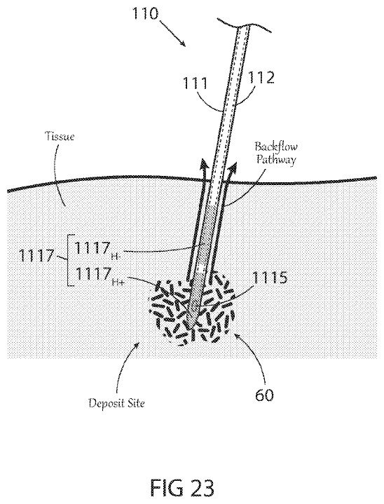

Referring now to FIG. 23, an anatomic view of an embodiment of an infusion device inserted into tissue is illustrated, consistent with the present inventive concepts. Infusion assembly 110 and other components of FIG. 23 can be of similar construction and arrangement to similar components described in reference to FIG. 1 and otherwise herein.

Infusion assembly 110 can be configured to prevent and/or at least limit backflow of a material, such as treatment material 60, that is delivered to the patient via infusion assembly 110 (e.g., infused into a deposit site). When infusing a material to a deposit site to treat target tissue (e.g., tissue of an organ of a patient), backflow can occur when the material exits the organ via a fluid path surrounding shaft 111 (e.g., the path of least resistance for treatment material 60 can comprise a fluid path between the tissue surrounding shaft 111 and shaft 111, such as a path created when shaft 111 is inserted through the tissue). In some embodiments, system 10 can be configured to limit backflow (e.g., in addition to anti-backflow configurations of infusion assembly 110), as described herein. The pathway created by the insertion of shaft 111 through the tissue can be referred to herein as a “backflow pathway”. While shaft 111 is positioned within the tissue (e.g., before shaft 111 is retracted after an infusion of treatment material 60), the backflow pathway comprises an initial backflow pathway BP1 comprising the fluid path between the outer surface of shaft 111 and the surrounding tissue as described hereabove. Once shaft 111 has been removed, the backflow pathway comprises a remaining pathway BP2 comprising the fluid path that remains due to tissue disruption caused by the insertion and/or removal of shaft 111.

Infusion assembly 110 can comprise shaft 111, such as a needle-like shaft, with lumen 112 passing therethrough. As described herein, infusion assembly 110 can comprise coating 1117. Coating 1117 can comprise one or more coatings, such as one or more coatings applied to one or more segments of shaft 111, such as one or more discrete and/or overlapping segments. Infusion assembly 110 can include port 1115, such as one or more side ports, for example as described in reference to FIG. 21 and otherwise herein.

In some embodiments, coating 1117 comprises a hydrophobic coating, coating 1117_{II-}, that surrounds (e.g., has been applied to the outer surface of) a segment of shaft 111 proximal to port 1115. Coating 1117_{H-} can comprise a hydrophobic coating selected from the group consisting of: a PTFE coating; a silicone coating; another hydrophobic material coating; and combinations of these. Coating 1117_{H-} can be configured to limit treatment material 60 from following the initial backflow pathway proximally along shaft 111. In some embodiments, the portion of shaft 111 that is distal to the portion comprising coating 1117_{H-} is more hydrophilic than the portion comprising coating 1117_{H-}, such that treatment material 60 is more likely to “cling” to the distal segment, and to infuse into tissue (e.g., instead of following the initial backflow pathway). In some embodiments, coating 1117 comprises a hydrophilic coating, coating 1117_{H+}. For example, shaft 111 can comprise a proximal segment (e.g., proximal to port 1115) comprising hydrophobic coating 1117_{H-}, and a more distal segment (e.g., a segment distal to and/or including port 1115) comprising hydrophilic coating 1117_{II+}. Coating 1117_{II+} can further increase the “cling” of treatment material 60 to the distal portion of shaft 111 (e.g., to maintain treatment material 60 proximate the target region and limit backflow while treatment material 60 is infused into the target region).

In some embodiments, application of coating 1117 (e.g., in a manufacturing process) comprises a surface treatment that is performed on a segment of shaft 111. In some embodiments, application of coating 1117 can include hydrofluoric acid etching and/or fluorosilane modification. For example, application of coating 1117 can include a pretreatment configured to increase the surface roughness of shaft 111 (e.g., hydrofluoric acid etching), followed by covalent modification with long-chain fluorosilanes, such as to form a self-assembled monolayer (SAM). In some embodiments, coating 1117, such as coating 1117 comprising a SAM coating, is configured to reduce the surface energy of shaft 111 (e.g., shaft 111 comprising as stainless-steel shaft). Coating 1117 can comprise one or more surface modifications, such as one or more modifications selected from the group consisting of: laser-induced periodic surface structures (LIPSS); a modification configured to produce nanospikes, such as by controlling the wetting state of stainless-steel with accumulated laser fluence; and combinations of these. In some embodiments, coating 1117 comprises a coating that is applied to shaft 111 during a clinical procedure. For example, shaft 111 can be “dipped” into a coating material prior to insertion of shaft 111 into the patient. In some embodiments, coating 1117 comprises a fluid that is applied to shaft 111 prior to insertion, such as a fluid comprising a microbubble contrast agent.

In some embodiments, coating 1117 comprises a friction reducing coating, such as hydrophobic coating 1117_{H-} described herein, or other friction reducing coating. A friction reducing coating can prevent or at least limit trauma to tissue as shaft 111 is inserted therethrough, such as to position port 1115 proximate a deposit site. One or more components and/or methods of system 10 can be configured to limit trauma to tissue, such as to prevent and/or at least limit likelihood of procedural complication, such as pancreatitis caused by tissue injury, and/or to limit the size and/or the lasting duration of the temporary backflow pathway that remains when infusion assembly 110 is removed from tissue (e.g., shaft 111 is retracted from the deposit site where treatment material 60 was delivered).

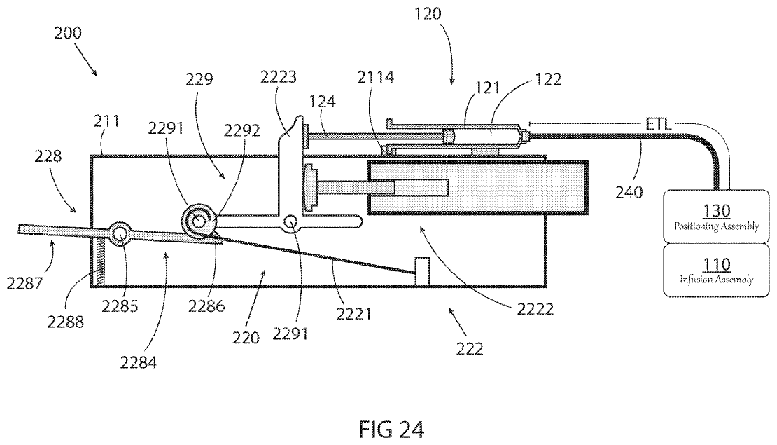

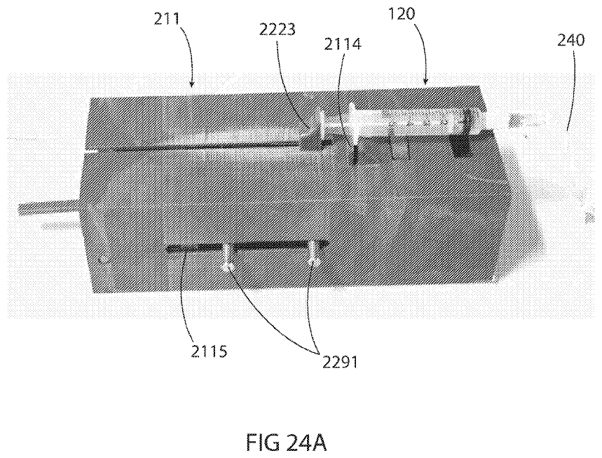

Referring now to FIGS. 24 and 24A, a schematic view of an embodiment of a drive assembly, and a photograph of the drive assembly are illustrated, respectively, consistent with the present inventive concepts. Drive assembly 200 and other components of FIGS. 24 and 24A can be of similar construction and arrangement to similar components described in reference to FIG. 1 and otherwise herein. Drive assembly 200 can be configured to operably attach to syringe assembly 120, and to provide a force to advance plunger 124, such as to drive plunger 124 within chamber 122, as described herein. Drive assembly 200 can be configured to control the flowrate of treatment material 60 from syringe assembly 120, through extension tube 240, positioning assembly 130, and/or infusion assembly 110, and into the patient at a location proximate a deposit site (e.g., a location where a distal portion of infusion assembly 110 has been inserted).

Extension tube 240 can fluidly connect syringe assembly 120 to infusion assembly 110, as described herein. Extension tube 240 comprises a length ETL. Length ETL can comprise a length sufficient to allow drive assembly 200 to be positioned remote from positioning assembly 130, such as when drive assembly 200 is positioned on a table or other surface of a clinical suite, and positioning assembly 130 comprises a handheld device that is held by the operator of system 10. In some embodiments, length ETL comprises a length of no more than 1 inch. In some embodiments, the embodiment of drive assembly 200 shown in FIG. 24 and FIG. 24A can be constructed and arranged to be attached to positioning assembly 130, such as when drive assembly 200 comprises a compact, handheld arrangement.

Drive assembly 200 can include housing 211 surrounding a drive mechanism, as described herein. Drive assembly 200 can include syringe adaptor 210 that includes an attachment assembly, such as clip 2114 shown. Clip 2114 can be configured to fixedly attach to barrel 121 of syringe assembly 120. Clip 2114 can be constructed and arranged to prevent axial motion of barrel 121 relative to drive assembly 200, such as to allow drive assembly 200 to drive plunger 124, as described herein. Clip 2114 can include one or more projections configured to slidingly receive (e.g., between two or more projections) and/or at least abut a flange of barrel 121, such as a projection that abuts the flange of barrel 121 and prevents translation of barrel 121 distally. As described and as shown in the figure, plunger 124 moves in a distal direction (e.g., to the right of the page as shown) when advancing through barrel 121, and a proximal direction when retracting (e.g., to the left of the page as shown) such as to draw material (e.g., treatment material 60) into chamber 122.

Drive assembly 200 can include an embodiment of motive assembly 220, such as an embodiment including biasing assembly 222. Motive assembly 220 can include translating assembly 229, that comprises pusher 2223, such that distal translation of translating assembly 229 causes pusher 2223 to drive plunger 124 into chamber 122. Housing 211 can comprise one or more alignment elements, slot 2115 shown, that can be configured to slidingly engage at least a portion of translating assembly 229 (e.g., one or more horizontal projections of translating assembly 229), such as to constrain the motion of translating assembly 229 to a single degree of freedom, such as to allow proximal and distal translation relative to the longitudinal axis of syringe barrel 121 and plunger 124. For example, translating assembly 229 can include two or more projections, pins 2291 shown, that extend through slot 2115, as shown.

Biasing assembly 222 can include spring 2221, such as a constant force spring. Spring 2221 can comprise a constant force spring that is wound about a rotating component of translating assembly 229, such as spool 2292 shown, that is constructed and arranged to allow spring 2221 to wind as translating assembly 229 is forced distally by spring 2221. Spring 2221 can be fixedly attached to a portion of housing 211, as shown, such that spring 2221 can apply a translating force to translating assembly 229. Alternatively, or additionally, spring 2221 can be wound about a portion of housing 211 (e.g., an inward projection of housing 211), with the free end of spring 2221 fixedly attached to translating assembly 229.

Motive assembly 220 can include locking assembly 228, such as a locking assembly that is constructed and arranged to lock translating assembly 229 in a proximal most position. Drive assembly 200 can be configured to deliver treatment material 60 from syringe assembly 120 with a constant flow and/or at a constant pressure after locking assembly 228 is unlocked, releasing translating assembly 229, as described herein. Locking assembly 228 can include a pivoting arm that is configured to lockingly engage translating assembly 229, such as arm 2284 that rotates about axle 2285. Arm 2284 can include locking portion 2286, and/or control portion 2287, where locking portion 2286 is configured to release translating assembly 229 when control portion 2287 is actuated (e.g., lifted as shown). Locking assembly 228 can include a biasing member, such as spring 2288 shown, configured to bias locking portion 2286 in a locked position (e.g., a locked position where locking portion 2286 is engaged with translating assembly 229). In some embodiments, motive assembly 220 can be set and/or reset (e.g., reset to an arrangement where translating assembly 229 is in a retracted and locked position after a first translation of translating assembly 229) by an operator physically retracting pins 2291 relative to housing 211, such that locking assembly 228 engages translating assembly 229. In some embodiments, locking assembly 228 comprises a ratcheting spool mechanism, such as a locking spool with a wound wire attached to translating assembly 229. The locking spool can comprise a handle (e.g., a cranking handle) configured to enable the operator to wind the spool, retracting translating assembly 229. The locking mechanism of the locking spool can be released to allow distal translation of translating assembly 229. Locking assembly 228 can comprise a sliding latching mechanism and/or a rotating latching mechanism.

Biasing assembly 222 can include damper 2222 that is configured to modify the force applied to translating assembly 229, and/or to otherwise control the flow rate of material 60 that is delivered by syringe assembly 120. Damper 2222 can comprise a hydraulic and/or other motion restricting assembly. Damper 2222 can comprise a constant force spring. In some embodiments, the force provided by damper 2222 and/or spring 2221 can be adjustable, such as to adjust the flow rate provided by drive assembly 200.

Drive assembly 200 can be configured to provide a constant flow rate of material from syringe assembly 120. For example, drive assembly 200 can provide a constant flow rate of at least 0.1 mL/min, such as at least 0.5 mL/min or 1.0 mL/min, and/or no more than 5.0 mL/min, such as no more than 3.0 mL/min or 2.00 mL/min, such as approximately 1 mL/min. Drive assembly 200 can be configured to provide a driving force (e.g., the force exerted on plunger 124 by pusher 2223) that creates a fluid pressure of at least 2.0 psi, such as at least 10 psi or 20 psi, and/or no more than 125 psi, such as no more than 75 psi or 50 psi, such as approximately 10 psi.

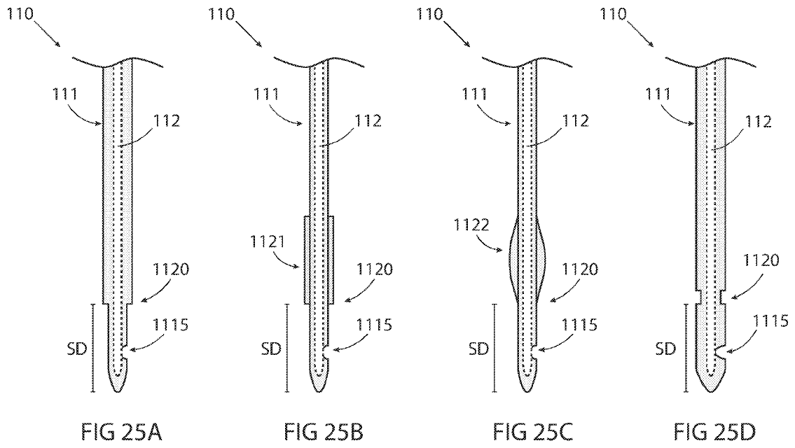

Referring now to FIGS. 25A-25D, various embodiments of infusion assemblies comprising stepped profiles are illustrated, consistent with the present inventive concepts. Infusion assembly 110 and other components of FIGS. 25A-D can be of similar construction and arrangement to similar components described in reference to FIG. 1 and otherwise herein. Infusion assembly 110 can be configured to prevent and/or at least limit backflow of a material, such as treatment material 60, that is delivered to the patient via infusion assembly 110 (e.g., infused into the target region via one or more deposit sites). Shaft 111 of infusion assembly can comprise a stepped profile, for example as shown in FIGS. 25A-D. The stepped profile of shaft 111 can be configured to limit backflow by limiting the initial backflow pathway described herein.

FIG. 25A shows an embodiment of shaft 111 comprising a multi-diameter shaft, where the outer diameter of the distal portion of shaft 111 comprises a smaller diameter than a proximal portion, as shown. Shaft 111 comprises a step, step 1120, that is formed at the transition from the first diameter to the second diameter, as shown. Step 1120 can be configured to reduce at least a portion of the temporary backflow pathway that is created when the distal portion of shaft 111 is inserted into tissue.

FIG. 25B shows an embodiment of shaft 111 comprising an over tube, sleeve 1121. Sleeve 1121 can comprise an outer diameter larger than the outer diameter of at least the distal portion of shaft 111, such that the distal end of sleeve 1121 forms step 1120 with shaft 111, as shown.

FIG. 25C shows an embodiment of shaft 111 comprising an adhesive bead, bead 1122. Bead 1122 can be applied to shaft 111 (e.g., in a manufacturing process) such that the distal end of bead 1122 forms step 1120 with shaft 111, as shown.

In some embodiments, step 1120 comprises an outer diameter of at least 0.001 in greater than the outer diameter of shaft 111, such as at least 0.002, 0.003, 0.004, and/or 0.005 in. In some embodiments, the outer diameter of step 1120 is no more than 0.0005 in greater than the outer diameter of shaft 111. In some embodiments, shaft 111 is machined (e.g., in a manufacturing process), such as to create a stepped profile, for example as shown in FIG. 25A. Alternatively, or additionally, an arrangement of two or more shafts of varying diameters can be assembled to form an arrangement as shown in FIG. 25A. In some embodiments, sleeve 1121 comprises a heat shrink type tube that is applied to shaft 111, as shown in FIG. 25B.

FIG. 25D shows an embodiment of shaft 111 comprising step 1120 comprising a reduced diameter portion of shaft 111. Step 1120 can be configured to allow an infused material to pool along shaft 111, such as to prevent additional material from continuing proximally along shaft 111. Step 1120 of FIG. 25D can be formed via a machining process (e.g., in a manufacturing process), such as a laser machining process.

In FIGS. 25A-D, step 1120 can be positioned a distance SD from the distal end of shaft 111. Distance SD can comprise a distance of at least 0.5 cm, and/or no more than 1.5 cm. Distance SD can comprise a distance of no more than 2 cm, such as no more than 1.75 cm. In some embodiments, step 1120 comprises two or more steps, such as two or more steps 1120, each positioned proximal to port 1115 and no more than 2 cm from the distal end of shaft 111.

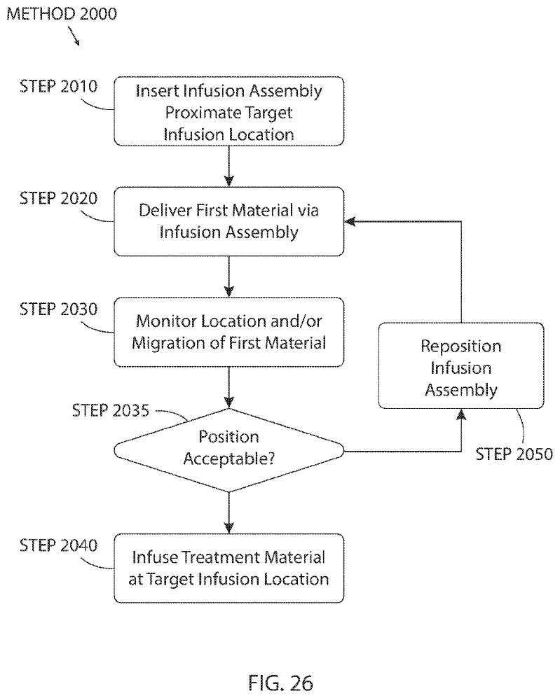

Referring now to FIG. 26, a method of limiting backflow during delivery of an injectate under visualization is illustrated, consistent with the present inventive concepts. FIG. 26 illustrates Method 2000 for infusing a material (e.g., treatment material 60) to a deposit site and into a target region. The material can be infused under visualization, for example EUS visualization described herein. Method 2000 can comprise a method configured to prevent and/or at least limit backflow of the infused material. The steps of Method 2000 can be performed with the various devices and components of system 10 described in reference to FIG. 1 and otherwise herein. Method 2000 can comprise a method of endoscopic ultrasound intraparenchymal infusion (EUS-IPI) of treatment material 60, as described herein.

In Step 2010, an infusion assembly, such as infusion assembly 110 of system 10, can be inserted into tissue proximate a deposit site. Infusion assembly 110 can be inserted using visual guidance, such as EUS visualization described herein.

In Step 2020, a first material (e.g., saline) can be delivered to the deposit site via infusion assembly 110. In some embodiments, at least 0.05 mL of the first material is delivered to the deposit site, such as approximately 1.0 mL. In some embodiments, the first material comprises a contrast agent, such as an ultrasonic contrast agent (e.g., a hyperechogenic agent). In some embodiments, the contrast agent comprises a microbubble contrast agent. In some embodiments, EUS imaging of Method 2000 comprises contrast harmonic imaging (CHI) that is configured for imaging microbubble contrast agents. In Step 2030, the location and/or migration of the first material within the tissue can be monitored, such as monitored by an operator of system 10 (e.g., using EUS visualization of the first material) and/or automatically or semi-automatically monitored by processing unit 310 of console 300 (e.g., when algorithm 315 is configured to monitor the migration of the first material via EUS visualization information).

In Step 2035, a determination is performed, to determine whether infusion assembly 110 is acceptably positioned for the infusion of a treatment material, such as treatment material 60 described herein. Infusion assembly 110 can be determined to be acceptably positioned when the first material is confirmed to have been delivered to the appropriate deposit site, and/or when limited migration (e.g., via backflow, as described herein) is observed of the first material. In some embodiments, migration of the first material can be identified by the visualization of pooling of the first material at a location offset from the deposit site, such as a location positioned proximal to the deposit site along infusion assembly 110. If it is determined that infusion assembly 110 is acceptably positioned, Method 2000 continues to Step 2040, wherein an infusion of treatment material 60 can be performed. Otherwise, Method 2000 continues to Step 2050, where infusion assembly 110 can be repositioned.

In Step 2040, treatment material 60 can be infused into the verified deposit site via the properly positioned infusion assembly 110. After a first infusion of treatment material 60, infusion assembly 110 can be removed from the patient (e.g., to end the clinical procedure), and/or infusion assembly 110 can be repositioned to a second deposit site, and Method 2000 can be repeated for verification of the new position of infusion assembly 110 and new deposit site. In some embodiments, infusion of treatment material 60 is also monitored using visualization, as described herein. Treatment material 60 can comprise a co-formulation of a therapeutic material with a contrast agent, such as an ultrasonic contrast agent (e.g., a hypoechogenic agent) configured to enhance the visualization of treatment material 60 during an infusion process. In some embodiments, a contrast agent, such as a microbubble contrast agent, is mixed with treatment material 60 (e.g., to create a co-formulation) immediately prior to infusion, such as immediately prior to loading syringe assembly 120 with treatment material 60 for infusion by depositing device 100. For example, a contrast agent can be mixed with treatment material 60 within no more than 30 min of infusion of material 60, such as no more than 20 min or 10 min.

In Step 2050, infusion assembly 110 can be repositioned, such as via further advancement, retraction and readvancement, and/or otherwise repositioned proximate the same deposit site and/or an alternate deposit site. After infusion assembly 110 is repositioned in Step 2050, Method 2000 can return to Step 2020 for another delivery of the first material, followed by verification, as described herein.

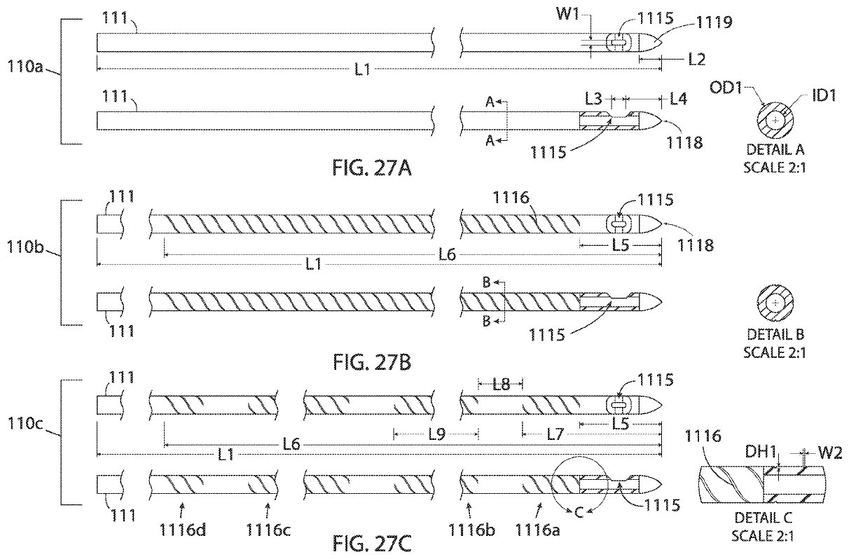

Referring now to FIGS. 27A-C, various embodiments of an infusion assembly are illustrated, consistent with the present inventive concepts. Infusion assembly 110 and other components of FIGS. 27A-C can be of similar construction and arrangement to similar components described in reference to FIG. 1 and otherwise herein. Shaft 111 of infusion assembly 110 can include one or more surface modifications, such as coating 1117 described herein, and/or one or more physical alterations to shaft 111, modification 1116. Embodiments of modification 1116 are shown in FIGS. 27B and 27C. In some embodiments, coating 1117 can be applied or otherwise arranged on shaft 111 in the same manner as modification 1116 is arranged in FIGS. 27B and/or 27C.

FIG. 27A shows a top view, a partial sectional side view, and a sectional view (Detail A) of shaft 111 of an embodiment of an infusion assembly, infusion assembly 110a. Infusion assembly 110a does not comprise a surface modification, such as coating 1117 and/or modification 1116 (referred to singly or collectively herein as modification 1116).

FIG. 27B shows a top view, a partial sectional side view, and a sectional view (Detail B) of shaft 111 of an embodiment of an infusion assembly, infusion assembly 110b. Infusion assembly 110b comprises modification 1116 comprising a helical etch extending along an outer longitudinal portion of shaft 111.

FIG. 27C shows a top view, a partial sectional side view, and a partial sectional expanded side view (Detail C) of shaft 111 of an embodiment of an infusion assembly, infusion assembly 110c. Infusion assembly 110c comprises modification 1116 comprising a helical etch extending along multiple discrete outer longitudinal portions of shaft 111.

In some embodiments, modification 1116 can comprise a modification configured to increase the echogenicity of shaft 111. For example, modification 1116 can comprise one or more projections from and/or one or more recesses on the outer surface of shaft 111, such as a helical etch, shown. Modification 1116 can comprise one or more helical etches, such as multiple overlapping helices that are spaced and rotated along and about shaft 111. For example, modification 1116 can comprise four helices with a pitch of 1 mm, each radially separated by 90°. Alternatively, or additionally, shaft 111 can comprise a single helix with a pitch of 0.25 mm. Modification 1116 can comprise a pattern selected from the group consisting of: rings; squiggles; “x” markings; circles; other geometric markings; and combinations of these. In some embodiments, modification 1116 comprises dimples and/or notches in the outer surface of shaft 111.

As shown in FIGS. 27A-C, shaft 111 comprises length L1. Tip 1119 comprises length L2. Port 1115 comprises width W1 and length L3. The distal end of port 1115 is located length L4 from distal end 1118 of shaft 111. In some embodiments, the distal most portion of modification 1116 is located proximal of distal end 1118, such as a length L5 from distal end 1118. Modification 1116 can extend length L6 from distal end 1118 of shaft 111. In some embodiments, as shown in FIG. 27C, two or more discrete portions of shaft 111 can include modification 1116, such as modifications 1116a-d shown. A distal most modification 1116, modification 1116a shown, can extend length L7 from distal end 1118 of shaft 111. One or more of modifications 1116a-d can be separated by a length L8 shown. One or more modifications of 1116a-c can comprise length L9. Modification 1116 can comprise an etching of shaft 111 (e.g., a helical etch, as described herein), wherein each etch comprises a width W2 and a depth DH1 (shown in Detail C). Shaft 111 comprises an outer diameter OD1, and an inner diameter ID1 (shown in Detail A).

In some embodiments, L1 comprises a length of at least 2 cm, such as at least 4 cm or 6 cm, and/or L1 comprises a length of no more than 15 cm, such as no more than 12 cm or 10 cm, such as approximately 8 cm. In some embodiments, W1 comprises a width of at least 0.05 mm, such as at least 0.75 mm, and/or W1 comprises a width of no more than 0.5 mm, such as no more than 0.25 mm or 0.2 mm, such as approximately 0.1 mm. In some embodiments, L2 comprises a length of at least 0.1 mm, such as at least 0.25 mm or 0.5 mm, and/or L2 comprises a length of no more than 1.5 mm, such as no more than 1 mm or 0.75 mm, such as approximately 0.5 mm. In some embodiments, L3 comprises a length of at least 0.1 mm, such as at least 0.2 mm or 0.3 mm, and/or L3 comprises a length of no more than 1 mm, such as no more than 0.75 mm or 0.05mm, such as approximately 0.35 mm. In some embodiments, L4 comprises a length of at least 0.1 mm, such as at least 0.25 mm or 0.5 mm, and/or L4 comprises a length of no more than 3 mm, such as no more than 2 mm or 1 mm, such as approximately 0.75 mm. In some embodiments, LA is greater than or equal to L2. In some embodiments, OD1 comprises a diameter of at least 0.31 mm (e.g., at least 30 G), and/or OD1 comprises a diameter of no more than 1.27 mm (e.g., no more than 18 G), such as approximately 0.4 mm (e.g., approximately 27 G). In some embodiments, ID1 comprises a diameter of at least 0.16 mm (e.g., at least 30 G), and/or ID1 comprises a diameter of no more than 0.84 mm (e.g., no more than 18 G), such as approximately 0.26 mm (e.g., approximately 27 G). In some embodiments, L5 comprises a length of at least 0.5 mm, such as at least 0.75 mm or 1 mm, and/or L5 comprises a length of no more than 5 mm, such as no more than 3 mm or 2 mm, such as approximately 1.5 mm. In some embodiments, L5 comprises a length that is greater than the cumulative length of L3 and L4. In some embodiments, L6 comprises a length of at least 10 mm, such as at least 15 mm or 20 mm, and/or L6 comprises a length of no more than 80 mm, such as no more than 40 mm or 30 mm, such as approximately 21.5 mm. In some embodiments, L6 comprises a length that is greater than L5. In some embodiments, L7 comprises a length of at least 2 mm, such as at least 3 mm or 4 mm, and/or L7 comprises a length of no more than 20 mm, such as no more than 10 mm or 7.5 mm, such as approximately 4.5 mm. In some embodiments, L8 comprises a length of at least 0.25 mm, such as at least 0.5 mm or 0.75 mm, and/or L8 comprises a length of no more than 10 mm, such as no more than 5 mm or 2.5 mm, such as approximately 1 mm. In some embodiments, L9 comprises a length of at least 1 mm, such as at least 2 mm or 3 mm, and/or L9 comprises a length of no more than 20 mm, such as no more than 10 mm or 5 mm, such as approximately 4 mm. In some embodiments, DH1 comprises a depth of at least 0.01 mm, such as at least 0.015mm or 0.02 mm, and/or DH1 comprises a depth of no more than 0.05 mm, such as no more than 0.04 mm or 0.03 mm, such as approximately 0.025 mm. In some embodiments, W2 comprises a width of at least 0.02 mm, such as at least 0.025 mm or 0.03 mm, and/or W2 comprises a width of no more than 0.1 mm, such as no more than 0.075 mm or 0.05 mm, such as approximately 0.04 mm.

In some embodiments, modification 1116 can cause an undesirable backflow condition, such as when modification 1116 comprises a helical etch through which material 60 can backflow from a target delivery location during an infusion, as described herein. As shown in FIG. 27C, modification 1116 can be applied to one or more segments of shaft 111, for example such that one or more unmodified segments block at least a portion of backflow material that may flow along modification 1116. Additionally, or alternatively, one or more segmented portions of modification 1116 can provide a visual marker (e.g., where the modified portions of shaft 111 are more effectively imaged by system 10 than unmodified portions, such as with EUS imaging). Lack of imaging effectiveness of unmodified portions of shaft 111 can provide visual markers, while a majority of the imaged portion of shaft 111 comprises modification 1116, such that a majority of the imaged portion of shaft 111 is more easily visualizable.

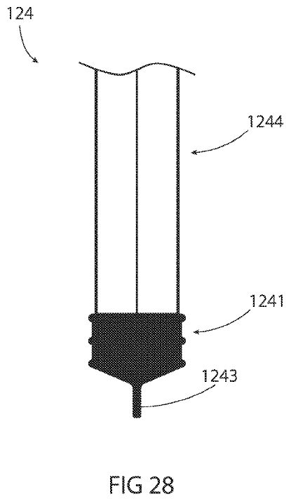

Referring now to FIG. 28, a side view of the distal end of an embodiment of a syringe plunger is illustrated, consistent with the present inventive concepts. Syringe assembly 120 and other components of FIG. 28 can be of similar construction and arrangement to similar components described in reference to FIG. 1 and otherwise herein. In some embodiments, one or more components of system 10 are constructed and arranged to minimize “dead-volume” of the system, or the volume of material 60 that remains within the fluid pathways of depositing device 100 following an infusion of material 60 (e.g., the volume of material 60 that is not infused into the patient, or otherwise wasted). System 10 comprises dead volume, volume DV. In some embodiments, volume DV of system 10 comprises a total volume of no more than 300 μL, such no more than 250 μL, 200 μL, 175 μL, 150 μL, or 125 μL.

Syringe assembly 120 can comprise plunger 124 that is configured to push material 60 from syringe barrel 121. FIG. 28 shows an embodiment of plunger 124 that is configured to minimize volume DV. Plunger 124 can include an elongate shaft, shaft 1244, that is attached to a sealing element, such as seal 1241 (as described herein), each shown. Shaft 1244 can be advanced and/or retracted, such as to advance and/or retract seal 1241 within barrel 121 (not shown). Seal 1241 is constructed and arranged to form a seal with the walls of barrel 121, such as to force material 60 from chamber 122 when seal 1241 is advanced within barrel 121. In some embodiments, seal 1241 comprises a shape and/or one or more features that are configured to minimize volume DV. For example, seal 1241 can comprise a projection, projection 1243 shown, that is slidingly received within a distal portion of syringe barrel 121 (e.g., to force material 60 through a narrow, connecting portion of the syringe). In some embodiments, the dead volume of syringe assembly 120 comprises a volume of less than 50 μL, such as less than 30 μL.

In some embodiments, system 10 includes one or more fluid connectors that fluidly attach two or more components of the flow path between syringe assembly 120 and infusion assembly 110. In some embodiments, system 10 comprises no more than two fluid connectors between syringe assembly 120 and infusion assembly 110, such as a first connector connecting syringe assembly 120 to a connecting tube (e.g., extension tube 240 not shown but described herein in reference to FIG. 24), and a second connector connecting the fluid tube to infusion assembly 110. As described herein, length ETL of extension tube 240 can be minimized to minimized volume DV. In some embodiments, syringe assembly 120 is attached directly to infusion assembly 110, for example when system 10 includes only a single connection between syringe assembly 120 and infusion assembly 110.

In some embodiments, the inner diameter of one or more lumens of the fluid path between syringe assembly 120 and the depositing site can comprise a maximum inner diameter IDM. In some embodiments, the maximum inner diameter IDM comprises a diameter of no more than 0.36 mm, such as a diameter of no more than 0.27 mm or 0.21 mm.

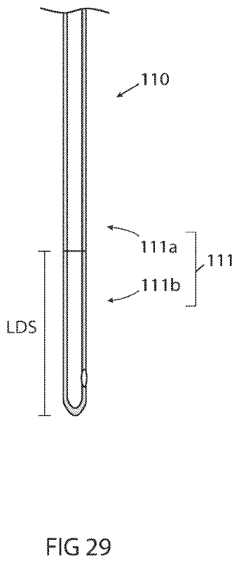

Referring now to FIG. 29, a sectional view of an embodiment of an infusion assembly comprising a multi-part configuration is illustrated, consistent with the present inventive concepts. Infusion assembly 110 and/or other components of FIG. 29 can be of similar construction and arrangement to similar components described in reference to FIG. 1 and otherwise herein. In some embodiments, infusion assembly 110 comprises a multi-part shaft, such as when shaft 111 comprises a proximal shaft, shaft 111a, and a distal shaft, shaft 111b, each shown. In some embodiments, shaft 111b comprises a needle-like shaft (e.g., a rigid shaft). Shaft 111a can comprise a tube (e.g., a flexible tube, that extends distally from shaft 111b. In some embodiments, shaft 111a comprises a larger inner diameter and/or a larger outer diameter than the inner and/or outer diameter, respectively, of shaft 111b. Shaft 111b can comprise length LDS. Length LDS can comprise a length of at least 5 cm, such as at least 6 cm, 7 cm, or 8 cm.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1.-94. (canceled)

95. An endoscopic-ultrasound (EUS) guided pancreatic infusion system comprising:
a handle configured to drive a treatment agent from a medicament container through an elongate fluid lumen sized to pass through a working channel of an endoscope;
a needle at a distal end of the elongate fluid lumen, the needle having a 27-gauge outer diameter, a conical distal tip, and a side port opening through a sidewall of the needle at a location proximal to the conical distal tip; and
an echogenic coating disposed on an external surface of a distal segment of the needle and configured to increase ultrasonic visibility of the needle during EUS imaging,
wherein the elongate fluid lumen is configured to deliver a medicament through the side port into target tissue located proximate to the side port.

96. The system of claim 95, further comprising an outer shaft slidingly receiving the elongate fluid lumen, the outer shaft configured to be longitudinally fixed with respect to an endoscope working channel, wherein distal translation of the elongate fluid lumen advances the needle through a distal opening of the outer shaft.

97. The system of claim 95, wherein the echogenic coating is applied proximal to and around the side port on the outer surface of the needle.

98. The system of claim 95, wherein the echogenic coating has a thickness of no more than 0.015 mm.

99. The system of claim 95, wherein the echogenic coating extends a length of 0.25-2.50 cm along the needle.

100. The system of claim 95, wherein a distal termination of the echogenic coating is within 2.50 cm of a distal end of the needle.

101. The system of claim 95, wherein the target tissue comprises pancreatic parenchyma, and wherein the needle comprises a hydrophobic coating on a segment proximal to the side port and a hydrophilic coating on a segment distal to the side port.

102. The system of claim 95, wherein application of the echogenic coating includes a surface-modification pretreatment selected from hydrofluoric-acid etching, fluorosilane functionalization to form a self-assembled monolayer (SAM), laser-induced periodic surface structures (LIPSS), or nanospike formation.

103. The system of claim 95, wherein the echogenic coating is applied intra-procedurally as a dip-applied fluid.

104. The system of claim 95, further comprising a positioning assembly having an outer shaft slidingly receiving the needle and configured for deployment from an endoscope working channel and extension through the stomach wall.

105. The system of claim 95, wherein the treatment agent comprises a gene-therapy composition including a viral vector, and one or more fluid-path components comprise a material selected to reduce viral adsorption.

106. The system of claim 95, wherein the elongate fluid lumen comprises a material including at least one of: cyclic olefin polymer (COP), polypropylene (PP), polyethylene terephthalate (PET), polyethylene (PE), polytetrafluoroethylene (PTFE), Tygon and nylon to reduce adsorption, and the needle comprises stainless steel.

107. The system of claim 95, wherein the needle includes a proximal step or reduced-diameter region configured to encourage pooling of infused material and limit proximal backflow along the needle track.

108. The system of claim 95, wherein the handle comprises a drive assembly configured to operatively engage a plunger of the medicament container within the handle and configured to control flow rate of the treatment agent into the target tissue.

109. The system of claim 108, wherein the drive assembly includes a mechanical drive mechanism configured to generate linear motion of an actuator, the actuator being configured to move the plunger of the medicament container.

110. The system of claim 109, wherein the mechanical drive mechanism is manually actuated via a mechanism on the handle.

111. The system of claim 110, wherein the mechanical drive mechanism comprises at least one constant-force spring operatively coupled to the actuator.

112. The system of claim 108, wherein the handle is configured to secure the medicament container in the handle.

113. The system of claim 109, wherein the mechanical drive mechanism includes a pusher rod or plunger coupler aligned with an axis of the medicament container.

114. The system of claim 108, wherein the elongate fluid lumen comprises a proximal end with a coupling mechanism configured to fluidically couple a medicament container to the needle.

115. The system of claim 95, wherein the handle comprises a coupling configured to secure the handle to an endoscope.

116. The system of claim 115, wherein the coupling is position-adjustable to adjust the length of the elongate fluid lumen disposed within the endoscope.

117. The system of claim 95, wherein the side port opening is less than 1 cm from a distal end of the needle.

118. An endoscopic-ultrasound (EUS) guided pancreatic infusion system comprising:
a handle configured to drive a treatment agent from a medicament container through an elongate fluid lumen sized to pass through a working channel of an endoscope;
a needle at a distal end of the elongate fluid lumen, the needle having a conical distal tip and a side port opening through a sidewall of the needle at a location proximal to the conical distal tip; and
an outer shaft slidingly receiving the elongate fluid lumen, the outer shaft configured to be longitudinally fixed with respect to an endoscope working channel, wherein distal translation of the elongate fluid lumen advances the needle through a distal opening of the outer shaft,
wherein the elongate fluid lumen is configured to deliver a medicament through the side port into target tissue located proximate to the side port.
